(19) United States
(12) Patent Application Publication
(10) Pub. No.: US 2010/0048699 A1
(43) Pub. Date: Feb. 25, 2010

(54) CHEMICAL COMPOUNDS

(75) Inventors: Juergen Harry Schaetzer, Basel (CH); Christoph Luthy, Basel (CH); William Lutz, Basel (CH); Donn Warwick Moseley, Berkshire (GB); Anthony Cornellius O'Sullivan, Basel (CH)

Correspondence Address: SYNGENTA CROP PROTECTION , INC.;PATENT AND TRADEMARK DEPARTMENT, 410 SWING ROAD, GREENSBORO, NC, 27409, US

(73) Assignees: SYNGENTA CROP PROTECTION, INC., Greensboro, NC (US); SYNGENTA LIMITED, Guilford, Surrey (GB)

(21) Appl. No.: 12/374,421

(22) PCT Filed: Jun. 29, 2007
(86) PCT No.: PCT/GB2007/002456

§ 371 Date: Sep. 16, 2009

(30) Foreign Application Priority Data
Jul. 21, 2006 (GB) 0614552.8

(51) Int. Cl. A01N 47/06; C07C 69/96; C07C 335/00; A01N 47/28

(52) U.S. Cl. 514/512; 558/275; 564/17; 514/580

(57) ABSTRACT

A compound of formula I

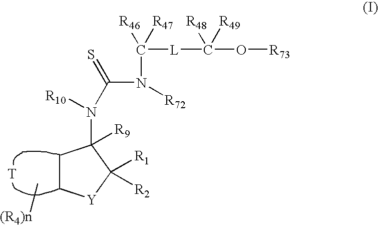

where the substituents have the meanings assigned to them in claim 1, compositions comprising a compound of formula (I) and the use of such compounds and/or compositions controlling insects, acarines, nematodes or molluscs.

The present invention relates to thiourea derivatives, to processes for preparing them, to insecticidal, acaricidal, molluscicidal and nematicidal compositions comprising them and to methods of using them to combat and control insect, acarine, mollusc and nematode pests.

The present invention therefore provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I)

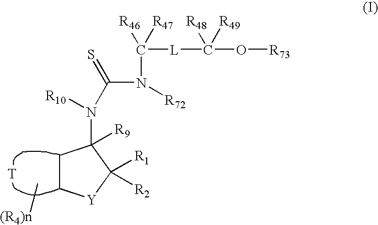

wherein

R⁷³ is hydrogen, G-, formyl, G-C(O)—, G-C(S)—, G-O—C(O)—, G-O—C(S)—, R⁷⁸R⁷⁹N—C(O)—, R⁷⁸R⁷⁹N—C(S)—, where R⁷⁸ and R⁷⁹ are independently H or G-, or R⁷⁸ and R⁷⁹ together with the N atom to which they are attached, form a group N═CRaRb, where Ra and Rb are H, C₁₋₆ alkyl or phenyl; or R⁷⁸ and R⁷⁹ together with the N atom to which they are attached form a five, six or seven membered ring which may contain one or two further hetero atoms selected from O, N or S and which may be optionally substituted by one to four C₁₋₆ alkyl groups;

R¹⁰ and R⁷² are independently hydrogen, hydroxy, amino, cyano, formyl, G-, G-O—, G-S—, G-S—S—, G-A-, R²⁴R²⁵N—, G-A-NR¹⁷, R²⁴R²⁵N—S—, R²⁴R²⁵N-A-, R¹⁸N═C(R¹⁹)—, G-O-A- or G-S-A-; where R²⁴ and R²⁵ are independently H or G-, or R²⁴ and R²⁵ together with the N atom to which they are attached, form a group N═CRaRb where Ra and Rb are H, C₁₋₆ alkyl or phenyl; or R²⁴ and R²⁵ together with the N atom to which they are attached form a five, six or seven membered ring which may contain one or two further hetero atoms selected from O, N or S and which may be optionally substituted by one to four C₁₋₆ alkyl groups or phenyl; R¹⁷ is H, G-, G-C(O)— or G-OC(O)—; R¹³ is H, OH, cyano, nitro, G-, G-O— or R³⁸R³⁹N—, where R³³ and R³⁹ are independently H or G-, or R³⁸ and R³⁹ together with the N atom to which they are attached, form a group N═CRaRb, where Ra and Rb are H, C₁₋₆ alkyl or phenyl; or R³⁸ and R³⁹ together with the N atom to which they are attached form a five, six or seven membered ring which may contain one or two further hetero atoms selected from O, N or S and which may be optionally substituted by one to four C₁₋₆ alkyl groups; R¹⁹ is H, cyano, G-, G-O—, G-S— or R⁴²R⁴³N—, where R⁴² and R⁴³ are independently H or G; or R⁴² and R⁴³ together with the N atom to which they are attached, form a group N═CRaRb, where Ra and Rb are H, C₁₋₆ alkyl or phenyl; or R⁴² and R⁴³ together with the N atom to which they are attached form a five, six or seven membered ring which may contain one or two further hetero atoms selected from O, N or S and which may be optionally substituted by one to four C₁₋₆ alkyl groups or phenyl;

L is a direct bond, CR⁷⁴R⁷⁵ or CR⁷⁴R⁷⁵CR⁷⁶R⁷⁷, where R⁷⁴, R⁷⁵, R⁷⁶R⁷⁷ independently hydrogen, OH, halogen, COOH, cyano, formyl, G-, GO-, GS-, G-C(O)—, G-C(S)—, G-O—C(O)—, G-O—C(S)—, R⁸⁰R⁸¹N—C(O)—, R⁸⁰R⁸¹N—C(S)—; or the groups R⁷⁴ and R⁷⁵ and/or R⁷⁶ and R⁷⁷ together with the carbon atom to which they are attached form a three to six membered ring, containing at least 2 carbon atoms and optionally containing one or two sulphur and/or one or two non-adjacent oxygen atoms or a group NR⁸², S(O)₂, S(O) or C(O) the ring being optionally substituted by C₁₋₆ alkyl or phenyl, where R⁸⁰ and R⁸¹ are independently H or G-, or R⁸⁰ and R⁸¹ together with the N atom to which they are attached, form a group N═CRaRb, where Ra and Rb are H, C₁₋₆ alkyl or phenyl; or R⁸⁰ and R⁸¹ together with the N atom to which they are attached form a five, six or seven membered ring which may contain one or two further hetero atoms selected from O, N or S and which may be optionally substituted by one to four C₁₋₆ alkyl groups; R⁸² is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷ and R²⁸ are independently H or G-, or R²⁷ and R²⁸ together with the N atom to which they are attached, form a group N═CRaRb where Ra and Rb are H, C₁₋₆ alkyl or phenyl; or R²⁷ and R²⁸ together with the N atom to which they are attached form a five, six or seven membered ring which may contain one or two further hetero atoms selected from O, N or S and which may be optionally substituted by one to four C₁₋₄ alkyl groups or phenyl; R²⁹ is H or G-; or two of the groups R⁷⁴, R⁷⁵, R⁷⁶ and R⁷⁷ attached to different atoms together with the atoms to which they are attached form a three to seven membered ring, that optionally contains one or two sulphur and/or one or two non-adjacent oxygen atoms or a group NR⁸², S(O)₂, S(O) or C(O), the ring being optionally substituted by C₁-C₆ alkyl or phenyl; where R⁸² has the meanings assigned to it above; R⁴³, R⁴⁷, R⁴⁸ and R⁴⁹ are each independently hydrogen, halogen, G-, G-C(O)—, G-C(S)—, G-O—C(O)—, G-O—C(S)—, R⁸³R⁸⁴N—C(O)—, R⁸³R⁸⁴N—C(S)—; where R³³ and R⁸⁴ are independently H or G-, or R⁸³ and R⁸⁴ together with the N atom to which they are attached, form a group N═CRaRb where Ra and Rb are H, C₁₋₆ alkyl or phenyl; or R⁸³ and R⁸⁴ together with the N atom to which they are attached form a five, six or seven membered ring which may contain one or two further hetero atoms selected from O, N or S and which may be optionally substituted by one to four C₁₋₆ alkyl groups or phenyl, or the groups R⁴⁶ and R⁴⁷ and/or R⁴⁸ and R⁴⁹ together with the carbon atom to which they are attached form a three to six membered ring, containing at least 2 carbon atoms and optionally containing one or two sulphur and/or one or two non-adjacent oxygen atoms or a group NR⁸⁵, where R⁸⁵ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷ and R²⁸ are independently H or G-, or R²⁷ and R²⁸ together with the N atom to which they are attached, form a group N═CRaRb where Ra and Rb are H, C₁₋₆ alkyl or phenyl; or R²⁷ and R²⁸ together with the N atom to which they are attached form a five, six or seven membered ring which may contain one or two further hetero atoms selected from O, N or S and which may be optionally substituted by one to four C₁₋₆ alkyl groups or phenyl; or two of the groups R⁴⁶, R⁴⁷, R⁴⁸, R⁴⁹, R⁷⁴, R⁷⁵, R⁷⁶ and R⁷⁷ attached to different atoms together with the atoms they are attached form a three to seven membered ring, that optionally contains one or two sulphur and/or one or two non-adjacent oxygen atoms or a group NR⁸⁵, where R⁸² and R⁸⁵ have the meanings assigned to them as above, or a group S(O)₂, S(O) or C(O) the ring being optionally substituted by C₁-C₆ alkyl or phenyl;

Y is O, S(O)ₘ, where m is 0, 1 or 2, NR³, SO₂—NR³, NR³—SO₂, NR³—O or O—NR³ where R³ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above; or Y is CR⁵R⁶, CR⁵R⁶—CR⁷R⁸, O—CR⁷R⁸, S(O)ₘ—CR⁷R⁸, NR³—CR⁷R⁸, CR⁵R⁶—O, CR⁵R⁶—S(O)ₘ or CR⁵R⁶—NR³, where R³ and m have the meanings assigned to them above, and R⁵, R⁶, R⁷ and R⁸ are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³, R²¹R²² N-A-O—R²¹R²²N-A-S, R²¹R²²NA NR²³—, G-O-A-O—, G-O-A-S—, G-O-A-NR²³—, G-S-A-O, G-S-A-NR²³— or R²⁰S(O)(═NR¹⁷)—, where R²¹ and R²² are independently H or G-, or R²¹ and R²² together with the N atom to which they are attached, form a group N═CRaRb, where Ra and Rb are H, C₁₋₆ alkyl or phenyl; or R²¹ and R²² together with the N atom to which they are attached form a five, six or seven membered ring which may contain one or two further hetero atoms selected from O, N or S and which may be optionally substituted by one to four C₁₋₆ alkyl groups or phenyl; R²³ is H or G- and R¹⁷ is as defined above; R²⁰ is C₁₋₆ alkyl, optionally substituted phenyl, optionally substituted benzyl; or two of the groups R⁵, R⁶, R⁷ and R⁸ attached to the same carbon atom are ═O, ═S, ═NR¹¹ or ═CR¹²R¹³, where R¹¹ is H, OH, nitro, cyano, formyl, formyloxy, G-, G-O—, G-A-, R³⁶R³⁷N—, G-C(O)—O—, G-C(O)—NR²⁶—, R³⁶R³⁷N—C(O)O—, G-O—C(O)O—, G-O—C(O)—NR²⁶—, where R³⁸, R³ and R²⁶ are independently H or G-, or R³⁶ and R³⁷ together with the N atom to which they are attached, form a group N═CRaRb where Ra and Rb are H, C₁₋₆ alkyl or phenyl; or R³⁶ and R³⁷ together with the N atom to which they are attached form a five, six or seven membered ring which may contain one or two further hetero atoms selected from O, N or S and which may be optionally substituted by one to four C₁₋₆ alkyl groups or phenyl, and R¹² and R¹³ are each independently H, halogen, nitro, cyano, formyl, formyloxy, G-, G-O—, G-S—, G-A-, R⁴⁰R⁴¹N—, R⁴⁰R⁴¹N-A-, G-O-A-, G-A-O—, R⁴⁰R⁴¹N-A-O—, R⁴⁰R⁴¹N-A-S—, G-O-A-O—, G-O-A-S—, G-O-A-NR³⁰—; where R⁴⁰, R⁴¹ and R³⁰ are independently H or G-, or R⁴⁰ and R⁴¹ together with the N atom to which they are attached, form a group N═CRaRb where Ra and Rb are H, C₁₋₆ E alkyl or phenyl; or R⁴⁰ and R⁴¹ together with the N atom to which they are attached form a five, six or seven membered ring which may contain one or two further hetero atoms selected from O, N or S and which may be optionally substituted by one to four C₁₋₆ alkyl groups or phenyl, or R¹² and R¹³ together with the carbon atom to which they are attached form a 3 to 6 membered carbocyclic ring; or the groups R⁵ and R⁶ or R⁷ and R⁸ together with the carbon atom to which they are attached form a three to six membered ring, containing at least 2 carbon atoms and optionally containing one or two sulfur and/or one or two non-adjacent oxygen atoms or a group NR¹⁴, where R¹⁴ is H, OH, cyano, formyl, G-, G-O—, C—S—, C-A-, R²⁷R²⁸N—, R²⁷R²⁸N-A-, G-O-A-, G-S-A-, G-A-NR²⁹—, R²⁷R²⁸ N-A-NR²⁹—, G-O-A-NR²⁹— or G-S-A-NR²⁹—, where R²⁷, R²⁸ and R²⁹ have the meanings assigned to them above, the ring being optionally substituted by one to four C₁-C₆alkyl groups or phenyl; or two of the groups R⁵, R⁶, R⁷ and R⁸ attached to different atoms together with the atoms they are attached form a three to seven membered ring, that optionally contains one or two sulfur and/or one or two non-adjacent oxygen atoms or a group NR¹⁴, where R¹⁴ is as defined above, or two of the groups R⁵, R⁶, R⁷ and R³ attached to adjacent atoms combine to form a bond;

the ring (T)

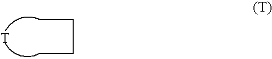

is a 5- or 6-membered aromatic or heteroaromatic ring;

R¹ and R² are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²²N-A-O—, R²¹R²²N-A-S—, R²¹R²²N-A-NR²³—, G-S-A-O—, G-O-A-S—, G-O-A-NR²³—, G-S-A-O—, G-S-A-NR²³—, or R²⁰S(O)(═NR¹⁷)—; where R¹⁷, R²⁰, R²¹, R²² and R²³ are as defined above, or two of the groups R¹ and R² attached to the same carbon atom are ═O, ═S, ═NR¹¹ or ═CR¹²R¹³, where R¹¹, R¹² and R¹³ are defined as above, or the groups R¹ and R² together with the same carbon atom to which they are attached form a three to six membered ring, containing at least 2 carbon atoms and optionally containing one or two sulfur and/or one or two non-adjacent oxygen atoms or a group NR¹⁴, where R¹⁴ is as defined above, the ring being optionally substituted by C₁-C₆ alkyl; or two of the groups R¹, R² and R⁷, R⁸ attached to different atoms together with the atoms they are attached form a three to seven membered ring, that optionally contains one or two sulfur and/or one or two non-adjacent oxygen atoms or a group NR¹⁴, where R¹⁴ is defined as above, the ring being optionally substituted by one or four C₁-C₆alkyl groups or phenyl; or two of the groups R¹, R², R⁵, R⁶, R⁷ and R⁸ attached to adjacent atoms combine to form a bond;

each R⁴ is independently OH, halogen, nitro, cyano, azido, rhodano, isothiocyanato, carboxy, formyl, formyloxy, G-, G-O—, G-S—, G-A-, R³¹R³²N—, R³¹R³²N-A-, G-O-A-, G-S-A-, G-A-O—, G-A-S—, G-A-NR³³—, R³¹R³²N-A-O—, R³¹R³²N-A-S—, R³¹R³²N-A-NR³³—, G-O-A-O—, G-O-A-S—, G-O-A-NR³³—, G-S-A-O—, G-S-A-NR³³—, R²⁰S(O)(═NR¹⁷)—, R¹⁸N═C(R¹⁹)—, R⁴⁴R⁴⁵P(O)— or R⁴⁴R⁴⁵P(S)—, where R¹⁷, R¹⁸, R¹⁹ and R²⁰ have the meanings assigned to them above, and R³¹, R³² and R³³ are independently H or G-, or R³¹ and R³² together with the N atom to which they are attached, form a group N═CRaRb, where Ra and Rb are H, C₁₋₆ alkyl or phenyl; or R³¹ and R³² together with the N atom to which they are attached form a five, six or seven membered ring which may contain one or two further hetero atoms selected from O, N or S and which may be optionally substituted by one to four C₁₋₆ alkyl groups or phenyl, and R⁴⁴ and R⁴⁵ are independently H, C₁₋₆alkyl, C₁₋₆-alkoxy, phenyl, phenoxy; or 2 adjacent groups R⁴ together with the carbon atoms to which they are attached form a 4, 5, 6 or 7 membered carbocyclic or heterocyclic ring which may be optionally substituted by C₁₋₆alkyl or halogen; or a group R⁴ together with a group R³, R⁵, R⁶ or R⁹ and the atoms to which they are attached form a 5-7 membered ring optionally containing an NR¹⁵ group where R¹⁵ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above, or containing an S or O atom, the ring being optionally substituted by one to four C₁-C₈ alkyl groups or phenyl;

n is 0, 1, 2, 3 or 4;

R⁹ is H, formyl, G-, G-A-, R³⁴R³⁵N-A-, where R³⁴ and R³⁵ are independently H or G-, or R³⁴ and R³⁵ together with the N atom to which they are attached, form a group N═CRaRb where Ra and Rb are H, C₁₋₆ alkyl or phenyl; or R³⁴ and R³⁵ together with the N atom to which they are attached form a five, six or seven membered ring which may contain one or two further hetero atoms selected from O, N or S and which may be optionally substituted by one to four C₁₋₆ alkyl groups or phenyl; or R⁹ is G-O-A- or G-S-A-; or R⁹ together with a group R¹, R², R³, R⁵, R⁶, R⁷ or R⁸ and the atoms to which they are attached may form a three to seven membered ring, that optionally may contain one or two sulfur and/or one or two non-adjacent oxygen atoms or a group NR¹⁶; where R¹⁶ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²³ and R²⁹ have the meanings assigned to them above,

G is optionally substituted C₁₋₁₂ alkyl, optionally substituted C₂₋₁₂ alkenyl, optionally substituted C₂₋₁₂ alkynyl, optionally substituted C₃₋₈ cycloalkyl, optionally substituted C₃₋₈ cycloalkenyl, optionally substituted aryl, optionally substituted heteroaryl or optionally substituted heterocyclyl;

### A is S(O), SO₂, C(O) or C(S);

or salts or N-oxides thereof,
with the proviso, that the compound of formula I is not: 1-(2-hydroxy-ethyl)-3-(5-methoxy-1,2,3,4-tetrahydro-naphthalen-1-yl)-thiourea, 1-(2-hydroxy-ethyl)-3-(1,2,3,4-tetrahydro-naphthalen-1-yl)-thiourea, methoxy-acetic acid 2-[3-(1,2,3,4-tetrahydro-naphthalen-1-yl)-thioureido]-ethyl ester, acetic acid 2-[3-(7-chloro-1,2,3,4-tetrahydro-naphthalen-1-yl)-thioureido]-ethyl ester, benzoic acid 2-[3-(7-chloro-1,2,3,4-tetrahydro-naphthalen-1-yl)-thioureido]-ethyl ester, acetic acid 2-[3-(7-bromo-1,2,3,4-tetrahydro-naphthalen-1-yl)-thioureido]-ethyl ester, benzoic acid 2-[3-(5-methoxy-1,2,3,4-tetrahydro-naphthalen-1-yl)-thioureido]-ethyl ester, 1-(7-bromo-1,2,3,4-tetrahydro-naphthalen-1-yl)-3-(2-hydroxy-ethyl)-thiourea, 1S-benzoic acid 2-(3-indan-1-yl-thioureido)-ethyl ester, nicotinic acid 2-(3-indan-1-yl-thioureido)-ethyl ester, nicotinic acid 2-(1-acetyl-3-indan-1-yl-thioureido)-ethyl ester, isonicotinic acid 2-(3-indan-1-yl-thioureido)-ethyl ester, pyridine-2-carboxylic acid 2-(3-indan-1-yl-thioureido)-ethyl ester, methoxy-acetic acid 2-(3-indan-1-yl-thioureido)-ethyl ester, 1-(2-hydroxy-ethyl)-3-indan-1-yl-thiourea, 1R-benzoic acid 2-(3-indan-1-yl-thioureido)-ethyl ester, acetic acid 2-[3-(6-methoxy-indan-1-yl)-thioureido]-ethyl ester, acetic acid 2-[3-(4-chloro-indan-1-yl)-thioureido]-ethyl ester, benzoic acid 2-[3-(4-chloro-indan-1-yl)-thioureido]-ethyl ester, 1-indan-1-yl-3-[2-(thiazol-2-yloxy)-ethyl]-thiourea, 4,5-dihydro-thiazole-2-carboxylic acid 2-(3-indan-1-yl-thioureido)-ethyl ester, 5-chloro-thiophene-2-carboxylic acid 2-((S)-3-indan-1-ylthioureido)-ethyl ester, furan-2-carboxylic acid 2-((S)-3-indan-1-ylthioureido)-ethyl ester, 3-trifluoromethyl-benzoic acid 2-((S)-3-indan-1-ylthioureido)-ethyl ester, 2-ethylsulfanyl-nicotinic acid 2-((S)-3-indan-1-ylthioureido)-ethyl ester, 2-methylsulfanyl-nicotinic acid 2-((S)-3-indan-1-ylthioureido)-ethyl ester, 3-cyano-benzoic acid 2-((S)-3-indan-1-ylthioureido)-ethyl ester, phenyl-acetic acid 2-((S)-3-indan-1-ylthioureido)-ethyl ester, 2-acetoxy-benzoic acid 2-((S)-3-indan-1-ylthioureido)-ethyl ester, diphenyl-acetic acid 2-((S)-3-indan-1-ylthioureido)-ethyl ester, 3-(2-chloro-phenyl)-5-methyl-isoxazole-4-carboxylic acid 2-((S)-3-indan-1-ylthioureido)-ethyl ester, 2-phenyl-butyric acid 2-((S)-3-indan-1-ylthioureido)-ethyl ester, cyclopentanecarboxylic acid 2-((S)-3-indan-1-ylthioureido)-ethyl ester, 3-methyl-butyric acid 2-((S)-3-indan-1-ylthioureido)-ethyl ester, naphthalene-2-carboxylic acid 2-((S)-3-indan-1-ylthioureido)-ethyl ester, 2,2-dimethyl-propionic acid 2-((S)-3-indan-1-ylthioureido)-ethyl ester, 3-phenyl-propionic acid 2-((S)-3-indan-1-ylthioureido)-ethyl ester, thiophen-2-yl-acetic acid 2-((S)-3-indan-1-ylthioureido)-ethyl ester, propionic acid 2-((S)-3-indan-1-ylthioureido)-ethyl ester, 3-methylsulfanyl-propionic acid 2-((S)-3-indan-1-ylthioureido)-ethyl ester, phenoxy-acetic acid 2-((S)-3-indan-1-ylthioureido)-ethyl ester, or 3,5-dimethyl-isoxazole-4-carboxylic acid 2-((S)-3-indan-1-ylthioureido)-ethyl ester.

The compounds of formula (I) as defined herein are novel and as such form a further aspect of the invention.

The compounds of formula (I) may exist as different geometric or optical isomers or in different tautomeric forms. One or more centres of chirality may be present, for example on the chiral carbon atoms CR¹R², CR⁴⁶R⁴⁷, CR⁴⁸R⁴⁹ and CR⁹ or a chiral carbon unit in the group G, or a chiral —S(O)— unit, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers of mixtures of isomers. Centres of tautomerisation may be present. This invention covers all such isomers and tautomers and mixtures thereof in all proportions as well as isotopic forms such as deuterated compounds.

Suitable acid addition salts include those with an inorganic acid such as hydrochloric, hydrobromic, sulfuric, nitric and phosphoric acids, or an organic carboxylic acid such as oxalic, tartaric, lactic, butyric, toluic, hexanoic and phthalic acids, or sulphonic acids such as methane, benzene and toluene sulphonic acids. Other examples of organic carboxylic acids include haloacids such as trifluoroacetic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

Each alkyl moiety either alone or as part of a larger group (such as G, or alkoxy, alkoxycarbonyl, alkylcarbonyl, alkylaminocarbonyl, dialkylaminocarbonyl) is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl. The alkyl groups are suitably C₁ to C₁₂ alkyl groups, but are preferably C₁-C₁₀, more preferably C₁-C₈, even more preferably C₁-C₆ and most preferably C₁-C₄ alkyl groups.

Ring or chain forming alkylen, alkenylen and alkinyl groups can optionally be further substituted by one or more halogen, C₁₋₃alkyl and/or C₁₋₃-alkoxy group.

When present, the optional substituents on an alkyl moiety (alone or as part of a larger group such as G, alkoxy, alkoxycarbonyl, alkylcarbonyl, alkylaminocarbonyl, dialkylaminocarbonyl) include one or more of halogen, nitro, cyano, rhodano, isothiocyanato, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkylsilyl(C₁₋₆)alkoxy, C₁₋₆ alkoxycarbonyl(C₁₋₁₀)alkoxy, C₁₋₁₀ haloalkoxy, aryl(C₁₋₄)-alkoxy (where the aryl group is optionally substituted), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio (where the aryl group is optionally substituted), C₃₋₇ cycloalkylthio (where the cycloalkyl group is optionally substituted with C₁₋₆ alkyl or halogen), tri(C₁₋₄)alkylsilyl(C₁₋₆)alkylthio, arylthio (where the aryl group is optionally substituted), C₁₋₆ alkylsulfonyl, C₁₋₆ haloalkylsulfonyl, C₁₋₆ alkylsulfinyl, C₁₋₆ haloalkylsulfinyl, arylsulfonyl (where the aryl group may be optionally substituted), tri(C₁₋₄)alkylsilyl, aryldi (C₁₋₄)alkylsilyl, (C₁₋₄)alkyldiarylsilyl, triarylsilyl, formyl, C₁₋₁₀ alkylcarbonyl, HO₂C, C₁₋₁₀ alkoxycarbonyl, aminocarbonyl, C₁₋₆alkylaminocarbonyl, di(C₁₋₆ alkyl)aminocarbonyl, N—(C₁— alkyl)-N—(C₁₋₃ alkoxy)aminocarbonyl, C₁₋₈ alkylcarbonyloxy, arylcarbonyloxy (where the aryl group is optionally substituted), di(C₁₋₆)alkylaminocarbonyloxy, aryl (itself optionally substituted), heteroaryl (itself optionally substituted), heterocyclyl (itself optionally substituted with C₁₋₆ alkyl or halogen), aryloxy (where the aryl group is optionally substituted), heteroaryloxy, (where the heteroaryl group is optionally substituted), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, C₂₋₆ alkenylcarbonyl, C₂₋₆ alkynylcarbonyl, C₃₋₆ alkenyloxycarbonyl, C₃₋₆ alkynyloxycarbonyl, aryloxycarbonyl (where the aryl group is optionally substituted) and arylcarbonyl (where the aryl group is optionally substituted). Furthermore a CH₂ moiety, which is part of the alkyl group may be substituted to an oxime or oximether such as —NOalkyl, —NOhaloalkyl and ═NOaryl (itself optionally substituted).

Alkenyl and alkynyl moieties can be in the form of straight or branched chains, and the alkenyl moieties, where appropriate, can be of either the (E)- or (Z)-configuration. Examples are vinyl, allyl and propargyl. Alkenyl and alkynyl moieties can contain one or more double and/or triple bonds in any combination. It is understood that allenyl and alkylinylalkenyl, as well as alkenylalkinyl, are included in these terms.

When present, the optional substituents on alkenyl or alkynyl include those optional substituents given above for an alkyl moiety.

In the context of this specification acyl is optionally substituted C₁₋₆ alkylcarbonyl (for example acetyl), optionally substituted C₂₋₆ alkenylcarbonyl, optionally substituted C₃₋₆ cycloalkylcarbonyl (for example cyclopropylcarbonyl, optionally substituted C₂₋₆ alkynylcarbonyl, optionally substituted arylcarbonyl (for example benzoyl) or optionally substituted heteroarylcarbonyl.

Halogen is fluoro, chlorine, bromine or iodine.

Haloalkyl groups are alkyl groups which are substituted with one or more of the same or different halogen atoms and are, for example, CF₃, CF₂Cl, CF₂H, CCl₂CH, FCH₂, ClCH₂, BrCH₂, CH₃CHF, (CH₃)₂CF, CF₃CH₂ or CHF₂CH₂.

In the context of the present specification the terms “aryl”, “aromatic ring” and “aromatic ring system” refer to ring systems which may be mono-, bi- or tricyclic. Examples of such rings include phenyl, naphthalenyl, anthracenyl, indenyl or phenanthrenyl. A preferred aryl group is phenyl. In addition, the terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl, pyrimidyl, triazinyl, thienyl, furyl, oxazolyl, isoxazolyl, 2,1,3-benzoxadiazole and thiazolyl.

The terms heterocycle and heterocyclyl refer to a non-aromatic preferably monocyclic or bicyclic ring systems containing up to 10 atoms including one or more (preferably one or two) heteroatoms selected from O, S and N. Examples of such rings include 1,3-dioxolane, oxetane, tetrahydrofuran, morpholine, thiomorpholin and piperazine.

When present, the optional substituents on heterocyclyl include C₁₋₆ alkyl and C₁₋₆ haloalkyl as well as those optional substituents given above for an alkyl moiety.

Cycloalkyl includes cyclopropyl, cyclobutyl, cyclopentyl and cyclohexyl. Cycloalkylalkyl is preferentially cyclopropylmethyl. Cycloalkenyl includes cyclopentenyl and cyclohexenyl. Carbocyclic rings include aryl, cycloalkyl, and cycloalkenyl groups.

When present, the optional substituents on cycloalkyl or cycloalkenyl include C₁₋₃ alkyl as well as those optional substituents given above for an alkyl moiety.

When present, the optional substituents on aryl or heteroaryl are selected independently, from halogen, nitro, cyano, rhodano, isothiocyanato, C₁₋₆ alkyl, C₁₋₆ haloalkyl, C₁₋₆ alkoxy-(C₁₋₆)alkyl, C₂₋₆ alkenyl, C₂₋₆ haloalkenyl, C₂₋₆ alkynyl, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkyl-silyl(C₁₋₆)alkoxy, C₁₋₆ alkoxycarbonyl(C₁₋₁₀)alkoxy, C₁₋₁₀ haloalkoxy, aryl(C₁₋₄)alkoxy (where the aryl group is optionally substituted with halogen or C₁₋₆ alkyl), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio, C₃₋₇ cycloalkylthio (where the cycloalkyl group is optionally substituted with C₁₋₆ alkyl or halogen), tri(C₁₋₄)-alkylsilyl(C₁₋₆)alkylthio, arylthio, C₁₋₆ alkylsulfonyl, C₁₋₆ haloalkylsulfonyl, C₁₋₆ alkylsulfinyl, C₁₋₆ haloalkylsulfinyl, arylsulfonyl, tri(C₁₋₄)alkylsilyl, aryldi(C₁₋₄)-alkylsilyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)-aminocarbonyl, N—(C₁₋₃ alkyl)-N—(C₁₋₃ alkoxy)aminocarbonyl, C₁₋₆ alkylcarbonyloxy, arylcarbonyloxy, di(C₁₋₆)alkylamino-carbonyloxy, aryl (itself optionally substituted with C₁₋₆ alkyl or halogen), heteroaryl (itself optionally substituted with C₁₋₆ alkyl or halogen), heterocyclyl (itself optionally substituted with C₁₋₆ alkyl or halogen), aryloxy (where the aryl group is optionally substituted with C₁₋₆ alkyl or halogen), heteroaryloxy (where the heteroaryl group is optionally substituted with C₁₋₆ alkyl or halogen), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, arylcarbonyl, (where the aryl group is itself optionally substituted with halogen or C₁₋₆ alkyl) or two adjacent positions on an aryl or heteroaryl system may be cyclised to form a 5, 6 or 7 membered carbocyclic or heterocyclic ring, itself optionally substituted with halogen or C₁₋₆ alkyl. Further substituents for aryl or heteroaryl include aryl carbonyl amino (where the aryl group is substituted by C₁₋₆ alkyl or halogen), (C₁₋₆)alkyloxycarbonylamino (C₁₋₆)alkyloxycarbonyl-N—(C₁₋₆)alkylamino, aryloxycarbonylamino (where the aryl group is substituted by C₁₋₆ alkyl or halogen), aryloxycarbonyl-N—(C₁₋₆)alkylamino, (where the aryl group is substituted by C₁₋₆ alkyl or halogen), arylsulphonylamino (where the aryl group is substituted by C₁₋₆ alkyl or halogen), arylsulphonyl-N—(C₁₋₆)alkylamino (where the aryl group is substituted by C₁₋₆ alkyl or halogen), aryl-N—(C₁₋₆)alkylamino (where the aryl group is substituted by C₁₋₆ alkyl or halogen), arylamino (where the aryl group is substituted by C₁₋₆ alkyl or halogen), heteroaryl amino (where the heteroaryl group is substituted by C₁₋₆ alkyl or halogen), heterocyclylamino (where the heterocyclyl group is substituted by C₁₋₆ alkyl or halogen), aminocarbonylamino, C₁₋₆ alkylaminocarbonyl amino, di(C₁₋₆)alkylaminocarbonyl amino, arylaminocarbonyl amino (where the aryl group is substituted by C₁₋₆ alkyl or halogen), aryl-N—(C₁₋₆)alkylaminocarbonylamino (where the aryl group is substituted by C₁₋₆ alkyl or halogen), C₁₋₆ alkylaminocarbonyl-N—(C₁₋₆)alkyl amino, di(C₁₋₆)alkylaminocarbonyl-N—(C₁₋₆)alkyl amino, arylaminocarbonyl-N—(C₁₋₆)alkyl amino (where the aryl group is substituted by C₁₋₆ alkyl or halogen) and aryl-N—(C₁₋₆)alkylaminocarbonyl-N—(C₁₋₆)alkyl amino (where the aryl group is substituted by C₁₋₆ alkyl or halogen).

For substituted phenyl moieties, heterocyclyl and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alkynyl, C₃₋₇ cycloalkyl, nitro, cyano, CO₂H, C₁₋₆ alkylcarbonyl, C₁₋₆ alkoxycarbonyl, aryl, heteroaryl, R⁵⁰R⁵¹N or R⁵²R⁵³NC(O), wherein R⁵⁰, R⁵¹, R⁵² and R⁵³ are, independently, hydrogen or C₁₋₆ alkyl.

Haloalkenyl groups are alkenyl groups which are substituted with one or more of the same or different halogen atoms.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

Preferably the optional substituents on an alkyl moiety include one or more of halogen, nitro, cyano, HO₂C, C₁₋₁₀ alkoxy (itself optionally substituted by C₁₋₁₀ alkoxy), aryl(C₁₋₄)alkoxy, C₁₋₁₀ alkylthio, C₁₋₁₀ alkylcarbonyl, C₃₋₅ cycloalkylcarbonyl, C₁₋₁₀ alkoxycarbonyl, C₁₋₆ alkylaminocarbonyl, di(C₁₋₆ alkyl)aminocarbonyl, (C₁₋₆)alkylcarbonyloxy, optionally substituted phenyl, heteroaryl, aryloxy, arylcarbonyloxy, heteroaryloxy, heterocyclyl, heterocyclyloxy, C₃₋₇ cycloalkyl (itself optionally substituted with (C₁₋₆)alkyl or halogen), C₃₋₇ cycloalkyloxy, C₅₋₇ cycloalkenyl, C₁₋₆ alkylsulfonyl, C₁₋₆ alkylsulfinyl, tri(C₁₋₄)alkylsilyl, tri(C₁₋₄)alkylsilyl(C₁₋₆)alkoxy, aryldi(C₁₋₄)alkylsilyl, (C₁₋₄)alkyldiarylsilyl and triarylsilyl.

Preferably the optional substituents on alkenyl or alkynyl include one or more of halogen, aryl, and C₃₋₇ cycloalkyl.

A preferred optional substituent for heterocyclyl is C₁₋₃ alkyl.

Preferably the optional substituents for cycloalkyl include halogen, cyano and C₁₋₆ alkyl.

The optional substituents for cycloalkenyl preferably include C₁₋₃ alkyl, halogen and cyano.

Y is preferably O, S, S(O), SO₂, NR³ or CR⁵R⁶, where R³, R⁵ and R⁶ are defined above. R³ is especially hydrogen, formyl, C₁₋₆ alkylcarbonyl, cyclopropylcarbonyl, C₁₋₆ alkoxycarbonyl, C₁₋₆ alkylsulfonyl, C₁₋₆ alkyl, C₁₋₆ haloalkyl, C₃₋₄ alkenyl, C₃₋₄ haloalkenyl, C₃₋₄ alkynyl, benzyl or phenyl (where the phenyl containing groups are optionally substituted by halogen, C₁₋₄ alkyl, C₁₋₄ alkoxy, C₁₋₄ haloalkyl, C₁₋₄ haloalkoxy, CN or NO₂). Especially-R⁵ and R⁶ are independently hydrogen, hydroxy, halogen, cyano, C₁₋₆ alkyl, C₁₋₆ haloalkyl, C₁₋₆ alkoxy(C₁₋₆)alkyl, phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), C₃₋₅ cycloalkyl, 1,3-dioxolan-2-yl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl, or two adjacent positions on the phenyl ring may be cyclised to form a 5, 6 or 7 membered carbocyclic or heterocyclic ring, itself optionally substituted with halogen), C₁₋₆ alkoxy, C₁₋₆ haloalkoxy, C₁₋₆ alkenyloxy, C₁₋₆ alkynyloxy, C₁₋₃ alkoxy(C₁₋₃)alkoxy, benzyloxy (wherein the phenyl is optionally substituted by halogen, C₁₋₄ alkyl, C₁₋₄ alkoxy, C₁₋₄ haloalkyl, C₁₋₄ haloalkoxy, CN, NO₂), C₁₋₆ alkylthio, C₁₋₆ haloalkylthio, NR⁵⁴R⁵⁵ (where R⁵⁴ and R⁵⁵ are independently hydrogen, C₁₋₆ alkyl, C₁₋₆ haloalkyl, C₁₋₄ alkoxy(C₁₋₄)alkyl, formyl, C₂₋₆ alkylcarbonyl or phenylcarbonyl (where the phenyl is optionally substituted by halogen, C₁₋₄ alkyl, C₁₋₄ alkoxy, C₁₋₄ haloalkyl, C₁₋₄ haloalkoxy, CN, NO₂)), or R⁵ and R⁶ together with the carbon atom to which they are attached form a three to six membered ring, that optionally may contain one or two sulfur or one or two not adjacent oxygen atoms or a group NR⁵⁶ (where R⁵⁶ is hydrogen, C₁₋₆ alkyl, C₁₋₆ haloalkyl, C₁₋₄ alkoxy(C₁₋₄)alkyl, formyl, C₂₋₆ alkylcarbonyl, or when R⁵ together with R¹ forms a bond, or either R⁵ or R⁶ together with R¹ or R² and the carbon atom they are attached form a three to six membered ring, that optionally may contain one or two not adjacent oxygen atoms, or R⁵ and R⁶ together form ═O, ═S, ═NR⁵⁷ or ═CR⁵⁸R⁵⁹, wherein R⁵⁷ is OH, optionally substituted C₁₋₆ alkoxy or C₁₋₄ alkylcarbonylamino, and R⁵⁸ and R⁵⁹ are independently H or C₁₋₅ alkyl.

More preferably Y is O or CR⁵R⁶, where R⁵ and R⁶ are hydrogen, hydroxy, fluoro, chloro, C₁₋₆ alkyl, C₁₋₆ haloalkyl, C₁₋₆ alkoxy, C₁₋₆ haloalkoxy, C₁₋₆ alkenyloxy, C₁₋₆ alkynyloxy, C₁₋₃ alkoxy(C₁₋₃)alkoxy or benzyloxy, or R⁵ and R⁶ together with the carbon atom to which they are attached form a three to six membered carbocyclic ring, or R⁵ and R¹ together form a bond, or R⁵ together with R¹ and the carbon atoms to which they are attached form a three to six membered carbocyclic ring, or R⁵ and R⁶ together form ═CR⁵⁸R⁵⁹, wherein R⁵⁸ and R⁵⁹ are independently H or C₁₋₆ alkyl.

Most preferably Y is CR⁵R⁶, where R⁵ and R⁶ are independently hydrogen, fluoro or methyl, especially hydrogen.

Preferably each R¹ and R² group is independently hydrogen, hydroxy, halogen, cyano, C₁₋₆ alkyl, C₁₋₆ haloalkyl, C₁₋₆ cyanoalkyl, C₁₋₆ hydroxyalkyl, C₁₋₆ alkoxy(C₁₋₆)alkyl, phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl), C₃₋₅ cycloalkyl, 1,3-dioxolan-2-yl, phenyl (which may be optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, C₁₋₆ alkoxycarbonyl), C₁₋₆ alkoxy, C₁₋₆ haloalkoxy, C₂₋₆ alkenyloxy, C₂₋₆ alkinyloxy, C₁₋₆ alkylthio, C₁₋₆ haloalkylthio, formyl, C₂₋₆ alkylcarbonyl, phenylcarbonyl (where the phenyl is optionally substituted by halogen, C₁₋₄ alkyl, C₁₋₄ alkoxy, C₁₋₄ haloalkyl, C₁₋₄ haloalkoxy, CN, NO₂), or R¹ and R² together are ═O, ═S, ═NR¹¹ or ═CR¹²R¹³, wherein R¹¹ is OH, C₁₋₆ alkoxy or C₁₋₆ alkylcarbonylamino, and R¹² and R¹³ are independently H, C₁₋₆ alkyl, or C₁₋₆ haloalkyl; or R¹ and R⁹ together with the carbon atom they are attached form a three to six membered ring, that optionally may contain one or two not adjacent oxygen atoms; or R¹ and R² together form a three to six membered ring, that optionally may contain one or two non-adjacent oxygen atoms. A preferred subset of this grouping of substituents excludes C₁₋₆ cyanoalkyl and C₁₋₆ hydroxyalkyl.

More preferably each R¹ and R² group is independently hydrogen, hydroxy, halogen, C₁₋₆ alkyl, C₁₋₆ haloalkyl, C₁₋₆ hydroxyalkyl, C₁₋₆ alkoxy, C₁₋₆ haloalkoxy, C₁₋₆ alkenyloxy, C₁₋₆ alkynyloxy, C₁₋₃ alkoxy(C₁₋₃)alkoxy or benzyloxy. A preferred subset of this grouping of substituents excludes C₁₋₆hydroxyalkyl.

Even more preferably each R¹ and R² group is independently hydrogen, hydroxy, fluoro or methyl. Most preferably each R¹ and R² group is independently hydrogen or methyl.

R⁹ is preferably hydrogen, C₁₋₆ alkyl, C₁₋₆ cyanoalkyl, C₁₋₆ haloalkyl, C₃₋₇ cycloalkyl(C₁₋₄)alkyl, C₁₋₆ alkoxy(C₁₋₆)alkyl, aryl(C₁₋₆)alkyl (wherein the aryl group may be optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino, arylcarbonyl), C₂₋₆ alkylcarbonyl, phenylcarbonyl (where the phenyl is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), C₁₋₆ alkoxycarbonyl, C(O)NR³⁴R³⁵ (where R³⁴ and R³⁵ are independently hydrogen, C₁₋₆ alkyl or C₁₋₆ haloalkyl or C₁₋₆ alkoxy(C₁₋₆)alkyl or R³⁴ and R³⁵ together with the N atom to which they are attached form a five, six or seven-membered ring containing an O or S atom); or R⁹ and R¹ together with the carbon atoms to which they are attached form a three to six membered ring, that may optionally contain one or two sulphur and/or one or two non-adjacent oxygen atoms.

More preferably R⁹ is independently hydrogen, C₁₋₆ alkyl, C₁₋₆ haloalkyl, C₃₋₇ cycloalkyl(C₁₋₄)alkyl, C₁₋₆ alkoxy(C₁₋₆)alkyl, aryl(C₁₋₆)alkyl (wherein the aryl group may be optionally substituted by halo, nitro, cyano, C₁₋₆ alkyl, C₁₋₆ haloalkyl, C₁₋₆ alkoxy), C₂₋₆ alkylcarbonyl, phenylcarbonyl (where the phenyl is optionally substituted by halogen, C₁₋₄ alkyl, C₁₋₄ alkoxy, C₁₋₄ haloalkyl), C₁₋₆ alkoxycarbonyl; or R⁹ and R¹ together with the carbon atoms to which they are attached form a three to six membered ring.

Most preferably R⁹ is independently hydrogen or methyl.

Preferably each R⁴ is independently halogen, nitro, cyano, C₁₋₈ alkyl, C₁₋₈ haloalkyl, cyano(C₁₋₆)alkyl, C₁₋₃ alkoxy(C₁₋₃)alkyl, C₂₋₆ alkynyl, C₃₋₆ cycloalkyl, C₁₋₃ alkyl-(C₃₋₆)cycloalkyl, C₃₋₆ cycloalkyl-(C₁₋₃)alkyl, phenyl (optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino), heterocyclyl (optionally substituted by halo, nitro, cyano, C₁₋₆ alkyl, C₁₋₆ haloalkyl, C₁₋₆ alkoxy or C₁₋₆ haloalkoxy), formyl, C₁₋₆ alkylcarbonyl, C₁₋₆ alkoxycarbonyl, C₁₋₆ alkylthiocarbonyl, C₁₋₆ alkoxythionocarbonyl, carbamoyl, C₁₋₆ alkylaminocarbonyl, di-C₁₋₆ alkylaminocarbonyl, thiocarbamoyl, C₁₋₆ alkylaminothionocarbonyl, di-C₁₋₆ alkylaminothionocarbonyl, C₁₋₈ alkoxy, C₁₋₆ haloalkoxy, phenoxy (optionally substituted by halogen, C₁₋₄ alkyl, C₁₋₄ alkoxy, C₁₋₄ haloalkyl, C₁₋₄ haloalkoxy, CN, NO₂, phenyl), heteroaryloxy (optionally substituted by halo, nitro, cyano, C₁₋₃ alkyl, C₁₋₃ haloalkyl, C₁₋₃ alkoxy or C₁₋₃ haloalkoxy), C₁₋₆ alkylcarbonyloxy, C₁₋₆ alkoxycarbonyloxy, C₁₋₆ alkylaminocarbonyloxy, di-C₁₋₆ (alkylaminocarbonyloxy, C₁₋₆ alkylaminothionocarbonyloxy, di-C₁₋₆ alkylaminothionocarbonyloxy, C₁₋₆ alkylthio, C₁₋₆ haloalkylthio, arylthio or heteroarylthio (where the aryl is optionally substituted by halogen, C₁₋₄ alkyl, C₁₋₄ alkoxy, C₁₋₄ haloalkyl, C₁₋₄ haloalkoxy, CN, NO₂ or phenyl), C₁₋₆ alkylcarbonylthio, C₁₋₆ alkylaminocarbonylthio, di-C₁₋₆ alkylaminocarbonylthio, di(C₁₋₈)alkylamino, C₁₋₆ alkylcarbonylamino, C₁₋₆ alkoxycarbonylamino, C₁₋₆ alkylaminocarbonylamino, di-C₁₋₆ alkylaminocarbonylamino, aminothionocarbonylamino, C₁₋₆ alkylaminothionocarbonylamino, di-C₁₋₆ alkylaminothionocarbonylamino; or 2 adjacent groups R⁴ together with the carbon atoms to which they are attached form a 4, 5, 6 or 7 membered carbocyclic or heterocyclic ring which may be optionally substituted by halogen; and n is 0, 1, 2 or 3, preferably 1, 2, or 3.

More preferably each R⁴ is independently halogen, nitro, cyano, C₁₋₈ alkyl, C₁₋₈ haloalkyl, cyano(C₁₋₆)alkyl, C₁₋₆ alkoxy(C₁₋₆)alkyl, C₂₋₆ alkynyl, heterocyclyl (optionally substituted by C₁₋₆ alkyl), C₁₋₈ alkoxy, C₁₋₆ haloalkoxy, phenoxy (optionally substituted by halo, cyano, C₁₋₃ alkyl or C₁₋₃ haloalkyl), heteroaryloxy (optionally substituted by halo, cyano, C₁₋₃ alkyl or C₁₋₃ haloalkyl), C₁₋₃ alkoxy, C₁₋₃ haloalkoxy, C₁₋₃ alkylthio, C₁₋₃ haloalkylthio, C₁₋₃ alksulfonyl, di(C₁₋₈)alkylamino, or 2 adjacent groups R⁴ together with the carbon atoms to which they are attached form a 4, 5, 6 or 7 membered carbocyclic or heterocyclic ring which may be optionally substituted by halogen; and n is 0, 1, 2 or 3, preferably 1, 2 or 3, more preferably 1 or 2.

Even more preferably each R⁴ is independently fluoro, chloro, bromo, C₁₋₄ alkyl or C₁₋₄ haloalkyl, most preferably fluoro, C₁₋₄ alkyl or C₁₋₄ haloalkyl; and n is 0, 1, 2 or 3, preferably 1 or 2.

Preferably at least one group R⁴ is positioned adjacent to the group Y. In particularly preferred embodiments this R⁴ group is selected from fluoro, fluoromethyl, difluoromethyl, trifluoromethyl and methyl.

In some embodiments R⁷³ is hydrogen, C₁₋₄ alkoxy-C₁₋₄ alkyl, C₁₋₄ haloalkoxy-C₁₋₄ alkyl, cyano-C₁₋₄ alkyl, 2-tetrahydropyranyl, 2-tetrahydrofuranyl, vinyl, cinnamyl (wherein the phenyl group is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl), C₃₋₆ alkenyl, C₃₋₆ alkinyl, aryldi(C₁₋₄)alkylsilyl, (C₁₋₄)alkyldiarylsilyl, formyl, C₁₋₁₂ alkylcarbonyl, where the alkyl group may be substituted by one or more halogen, C₁₋₆ alkoxy, C₁₋₆ alkylthio, C₁₋₆ alkoxycarbonyl, C₁₋₆ alkylcarbonyloxy, C₃₋₆ cycloalkyl, phenyl or phenoxy (wherein the phenyl group is optionally substituted by halogen, C₁₋₃ alkyl, C₁₋₃ haloalkyl, C₁₋₃ alkoxy, cyano, nitro); C₂₋₁₂ alkenylcarbonyl, where alkenyl may be substituted by Halogen, C₁₋₆alkoxycarbonyl or phenyl (that itself may be substituted by halogen, C₁₋₃ alkyl, C₁₋₃ haloalkyl, C₁₋₃ alkoxy, cyano or nitro); C₁₋₆ alkylthiocarbonyl, C₃₋₆ cycloalkylcarbonyl, adamantylcarbonyl, arylcarbonyl or heteroarylcarbonyl (wherein aryl or heteroaryl is optionally substituted by halogen, C₁₋₄ alkyl, C₁₋₄ alkoxy, C₁₋₄ haloalkyl, C₁₋₄ haloalkoxy, cyano or nitro); C₁₋₆ alkoxycarbonyl, C₁₋₆ alkylthiocarbonyl, C₁₋₆ alkoxythionocarbonyl, C₁₋₆ alkylthiothionocarbonyl, benzyloxycarbonyl, phenoxycarbonyl or phenylthiocarbonyl (wherein the phenyl group is optionally substituted by halogen, C₁₋₃ alkyl, C₁₋₃ haloalkyl, C₁₋₃ alkoxy, cyano, nitro); R⁷⁸R⁷⁹N—C(O)— or R⁷⁸R⁷⁹N—C(S)—, wherein R⁷⁸ and R⁷⁹ are independently hydrogen, C₁₋₆ alkyl, C₃₋₆ alkenyl, C₃₋₆ alkinyl or phenyl (which is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl.

In some embodiments R⁷³ is preferably C₁₋₄ alkoxy-C₁₋₄ alkyl, C₁₋₄ haloalkoxy-C₁₋₄ alkyl, cyano-C₁₋₄ alkyl, 2-tetrahydropyranyl, 2-tetrahydrofuranyl, cinnamyl (wherein the phenyl group is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl); aryldi(C₁₋₄)alkylsilyl, C₁₋₄ alkyldiarylsilyl, C₁₋₁₂ alkylcarbonyl, where the alkyl group is substituted by C₁₋₆ alkoxycarbonyl, C₁₋₆ alkylcarbonyloxy, C₃₋₆ cycloalkyl, phenyl or phenoxy (wherein phenyl group a are optionally substituted by halogen, C₁₋₃ alkyl, C₁₋₃ haloalkyl, C₁₋₃alkoxy, cyano or nitro); C₃₋₆ cycloalkylcarbonyl, adamantylcarbonyl, arylcarbonyl or heteroarylcarbonyl (wherein aryl or heteroaryl is substituted by C₁₋₄ haloalkyl, C₁₋₄ haloalkylthio, di-C₁₋₄-alkylamino, benzoyloxymethyl, phenyl or phenylsulfonyl (wherein the phenyl group is optionally substituted by halogen, C₁₋₃ alkyl, C₁₋₃ haloalkyl, C₁₋₃ alkoxy, cyano or nitro); naphthylcarbonyl, (2,3-dihydro-benzofuranyl)carbonyl, (2,2-difluoro-benzo[1,3]dioxolyl)carbonyl, phenoxycarbonyl or phenylthiocarbonyl (wherein phenyl groups are optionally substituted by halogen, C₁₋₃ alkyl, C₁₋₃ haloalkyl, C₁₋₃ alkoxy, cyano or nitro); C₂₋₁₂ alkenylcarbonyl, where alkenyl is substituted by C₁₋₆alkoxycarbonyl or phenyl (wherein the phenyl group is optionally substituted by halogen, C₁₋₃ alkyl, C₁₋₃ haloalkyl, C₁₋₃alkoxy, cyano or nitro); benzyloxycarbonyl (wherein the phenyl group is optionally substituted by halogen, C₁₋₃ alkyl, C₁₋₃ haloalkyl, C₁₋₃ alkoxy, cyano or nitro); R⁷¹R⁷⁹N—C(O)— or R⁷³R⁷⁹N—C(S)—, wherein R⁷⁸ and R⁷⁹ are independently hydrogen, C₃₋₆ alkenyl or phenyl (wherein the phenyl group is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl); or R⁷⁸ and R⁷⁹ together with the N atom to which they are attached form a five, six or seven membered ring which may contain one or two further hetero atoms selected from O, N or S and which may be optionally substituted by one to four C₁₋₆ alkyl groups.

Preferably R⁷³ is hydrogen, C₃₋₆ alkenyl, C₃₋₆ alkinyl, formyl, C₁₋₆ alkylcarbonyl, C₁₋₆ alkylthiocarbonyl, aryl- or heteroarylcarbonyl (where aryl or heteroaryl are optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, C₁₋₆ alkoxythionocarbonyl, C₁₋₆ alkylthiothionocarbonyl, R⁷⁸R⁷⁹N—C(O)— or R⁷⁸R⁷⁹N—C(S)—, wherein R⁷⁸ and R⁷⁹ are independently hydrogen, C₁₋₆ alkyl, C₃₋₆ alkenyl, C₃₋₆ alkinyl or phenyl (which is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl);

More preferably R⁷³ is hydrogen, formyl, C₁₋₆ alkylcarbonyl, phenylcarbonyl (where phenyl is optionally substituted by halogen, C₁₋₄ alkyl, C₁₋₃ alkoxy, C₁₋₃ haloalkyl, C₁₋₃ haloalkoxy, cyano or nitro), C₁₋₆ alkoxycarbonyl, R⁷⁸R⁷⁹N—C(O)— or R⁷⁸R⁷⁹N—C(S)—, wherein R⁷⁸ and R⁷⁹ are independently hydrogen, C₁₋₆ alkyl, C₃₋₆ alkenyl, C₃₋₆ alkinyl or phenyl (which is optionally substituted by halogen, C₁₋₄ alkyl, C₁ alkoxy, C₁₋₃ haloalkyl, C₁₋₃ haloalkoxy, cyano, nitro, C₁— alkylsulfonyl or C₁₋₃ alkoxycarbonyl);

Most preferably R⁷³ is hydrogen.

In some embodiments R¹⁰ and R⁷² are independently hydrogen, hydroxy, amino, cyano, C₁₋₆ alkyl, C₁₋₆ haloalkyl, C₁₋₆ alkoxy(C₁₋₆)alkyl, C₃₋₆-alkenyl, C₃₋₆alkinyl, aryl(C₁₋₃)alkyl or heteroaryl(C₁₋₃)alkyl (wherein aryl or heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₄ alkylsulfonyl or C₁₋₆ to alkoxycarbonyl); C₃₋₅ cycloalkyl-C₁₋₃alkyl, C₃₋₅ cycloalkyl, aryl or heteroaryl (wherein aryl or heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl and C₁₋₆ alkoxycarbonyl), C₁₋₆ alkylthio, C₁₋₆ haloalkylthio, C₁₋₆ alkyldithio, C₁₋₆ alkylthiosulfinyl, formyl, C₁₋₁₂ alkylcarbonyl, C₂₋₁₂ alkenylcarbonyl, where alkenyl is substituted by C₁₋₁₂ alkoxycarbonyl or phenyl (wherein the phenyl group is optionally substituted by halogen, C₁₋₃ alkyl, C₁₋₃ haloalkyl, C₁₋₃ alkoxy, cyano and/or nitro); C₃₋₆ cycloalkylcarbonyl, aryl- or heteroarylcarbonyl (wherein aryl or heteroaryl is optionally substituted by halogen, C₁₋₄ alkyl, C₁₋₄ alkoxy, C₁₋₄ haloalkyl, C₁₋₄ haloalkoxy, cyano and/or nitro), napthylcarbonyl, C₁₋₆ alkoxycarbonyl, C₁₋₆ alkylthiocarbonyl, C₁₋₆ alkylcarbonylamino, R²⁴R²⁵N—, R²⁴R²⁵N—S— or R²⁴R²⁵N-A-, wherein R²⁴ and R²⁵ are preferably hydrogen or C₁₋₆alkyl and A is SO₂, C(O) or C(S);

In some embodiments R¹⁰ and R⁷² are independently preferably hydroxy, amino, phenyl(C₂₋₃)alkyl (wherein the phenyl group is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl and/or C₁₋₆ alkoxycarbonyl), heteroaryl(C₁₋₃)alkyl (wherein heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₅ alkylsulfonyl or C₁₋₆ alkoxycarbonyl); C₃₋₅ cycloalkyl-C₁₋₃alkyl, heteroaryl (wherein heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl), C₁₋₆ alkylthio, C₁₋₆ haloalkylthio, C₁₋₆ alkyldithio, C₁₋₆ alkylthiosulfinyl, C₇₋₁₂ alkylcarbonyl, C₇₋₁₂ alkenylcarbonyl, cinnamylcarbonyl, (wherein the phenyl group is optionally substituted by halogen, C₁₋₃ alkyl, C₁₋₃ haloalkyl, C₁₋₃ alkoxy, cyano and/or nitro); aryl- or heteroarylcarbonyl (wherein aryl or heteroaryl is substituted by halogen, C₁₋₄ alkyl, C₁₋₄ alkoxy, C₁₋₄ haloalkyl, C₁₋₄ haloalkoxy, cyano and/or nitro); napthylcarbonyl, (2,2-difluoro-benzo[1,3]dioxolyl)-carbonyl, C₁₋₆ alkylcarbonylamino, R²⁴R²⁵N—, R²⁴R²⁵N—S— or R²⁴R²⁵N-A-, wherein R²⁴ and R²⁵ are preferably hydrogen or C₁₋₆alkyl and A is SO₂ or C(S).

Preferably R¹⁰ and R⁷² are independently hydrogen, hydroxy, amino, cyano, C₁₋₆ alkyl, C₁₋₆ haloalkyl, C₁₋₆ alkoxy(C₁₋₆)alkyl, C₃₋₆-alkenyl, C₃₋₆alkinyl, phenyl(C₁₋₃)alkyl (wherein the phenyl group is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl), C₃₋₅ cycloalkyl-C₁₋₃alkyl, C₃₋₅ cycloalkyl, aryl or heteroaryl (wherein aryl or heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl, C₁₋₆ alkoxycarbonyl), C₁₋₆ alkylthio, C₁₋₆ haloalkylthio, C₁₋₆ alkyldithio, C₁₋₆ alkylthiosulfinyl, formyl, C₁₋₆ alkylcarbonyl, aryl- or heteroarylcarbonyl (wherein aryl or heteroaryl is optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, C₁₋₆ alkylcarbonylamino, R²⁴R²⁵N—, R²⁴R²⁵N—S— or R²⁴R²⁵N-A-, wherein R²⁴ and R²⁵ are preferably hydrogen or C₁₋₆alkyl and A is SO₂, C(O) or C(S);

More preferably one of R¹⁰ or R⁷² is hydrogen and the other is hydrogen, amino, cyano, C₁₋₆ alkyl, C₁₋₆ haloalkyl, C₁₋₆ alkoxy(C₁₋₃)alkyl, C₃₋₆ alkenyl, C₃₋₆ alkinyl, phenyl-(C₁₋₁₂) alkyl (wherein phenyl is optionally substituted by halogen, C₁₋₄ alkyl, C₁₋₃ alkoxy, C₁₋₃ haloalkyl, C₁₋₃ haloalkoxy, cyano, nitro, C₁₋₃ alkylsulfonyl or C₁₋₄ alkoxycarbonyl), C₃₋₅ cycloalkyl-(C₁₋₃)alkyl, C₃₋₅ cycloalkyl, furyl, phenyl (which may be optionally substituted by halogen, C₁₋₄ alkyl, C₁₋₃ alkoxy, C₁₋₃ haloalkyl, C₁₋₃ haloalkoxy, cyano, nitro, C₁₋₃ alkylsulfonyl, or C₁₋₄ alkoxycarbonyl), C₁₋₆ alkylthio, C₁₋₆ haloalkylthio, C₁₋₆ alkyldithio, C₁₋₆ alkylthiosulfinyl, formyl, C₁₋₆ alkylcarbonyl, phenylcarbonyl (where phenyl is optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, C₁₋₆ alkylcarbonylamino, R²⁴R²⁵N—, R²⁴R²⁵N—S— or R²⁴R²⁵N-A-, wherein R²⁴ and R²⁵ are preferably hydrogen or C₁₋₆ alkyl and A is SO₂, C(O) or C(S).

Most preferably R¹⁰ and R⁷² are both hydrogen.

Preferably L is a direct bond, CR⁷⁴CR⁷⁵ or CR⁷⁴R⁷⁵CR⁷⁶R⁷⁷, wherein R⁷⁴, R⁷⁵, R⁷⁶ and R⁷⁷ are independently hydrogen, halogen, cyano, C₁₋₆ alkyl, C₁₋₆ haloalkyl, hydroxyl(C₁₋₆)alkyl, C₁₋₆ alkoxy(C₁₋₆)alkyl, phenyl(C₁₋₃)alkyl (wherein the phenyl group may be optionally substituted by halogen, C₁₋₄ alkyl, C₁₋₄ alkoxy, C₁₋₄ haloalkyl, C₁₋₄ haloalkoxy, cyano; nitro, aryl, heteroaryl, C₁₋₆ alkylsulfonyl, C₁₋₆ alkoxycarbonyl), C₃₋₆ cycloalkyl-C₁₋₃alkyl, C₃₋₆ cycloalkyl, 1,3-dioxolan-2-yl, phenyl (which is optionally substituted by halogen, C₁₋₄ alkyl, C₁₋₄ alkoxy, C₁₋₄ haloalkyl, C₁₋₄ haloalkoxy, cyano, nitro, C₁₋₅ alkylsulfonyl or C₁₋₆ alkoxycarbonyl), C₁₋₆ alkoxy, C₁₋₆ haloalkoxy, C₃₋₆ alkenyloxy, C₃₋₆ alkinyloxy, C₁₋₃ alkoxy(C₁₋₃)alkoxy, benzyloxy (wherein phenyl is optionally substituted by halogen, C₁₋₄ alkyl, C₁₋₄ alkoxy, C₁₋₄ haloalkyl, C₁₋₄ haloalkoxy, cyano or nitro), C₁₋₆ alkylthio, C₁₋₆ haloalkylthio, formyl, C₁₋₆ alkylcarbonyl, phenylcarbonyl (wherein phenyl is optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, R⁸⁰R⁸¹N—C(O)— or R⁸⁰R⁸¹N—C(S)—, wherein R⁸⁰ and R⁸¹ are independently hydrogen, C₁₋₆ alkyl, C₃₋₆ alkenyl C₃₋₆ alkinyl or phenyl (which is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl); or the groups R⁷⁴ and R⁷⁵ and/or R⁷⁶ and R⁷⁷ together with the carbon atom to which they are attached form a three to six membered ring, containing at least 2 carbon atoms and optionally containing a sulphur atom, an oxygen atom, S(O), S(O)₂ or a group NR⁸², wherein R⁸² is hydrogen, C₁₋₆ alkyl, C₂₋₆ alkenyl, C₃₋₆ alkinyl or benzyl (which may be optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl); the ring being optionally substituted by one or two C₁₋₃alkyl groups; or two of the groups R⁷⁴, R⁷⁵, R⁷⁵ and R⁷⁷ attached to different atoms together with the atoms to which they are attached form a three to six membered ring, that optionally contains a sulphur atom, an oxygen atom, S(O), S(O)₂, C(O) or a group NR⁸², and the ring being optionally substituted by one or two C₁₋₃ alkyl groups.

More preferably L is a direct bond or CR⁷⁴R⁷⁵, wherein each R⁷⁴ and R⁷⁵ are independently hydrogen, halogen, cyano, C₁₋₆ alkyl, C₁₋₆ haloalkyl, hydroxyl(C₁₋₆)alkyl, C₁₋₆ alkoxy(C₁₋₆)alkyl, phenyl(C₁₋₂)alkyl (wherein phenyl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl, C₁₋₆ alkoxycarbonyl), C₃₋₆cycloalkyl-C₁₋₃alkyl, C₃₋₅ cycloalkyl, 1,3-dioxolan-2-yl, phenyl (which is optionally substituted by halogen, C₁₋₄ alkyl, C₁₋₄ alkoxy, C₁₋₄ haloalkyl; C₁₋₄ haloalkoxy, cyano, nitro, C₁₋₆ alkylsulfonyl or C₁₋₆ alkoxycarbonyl), C₁₋₆ alkoxy, C₁₋₆ haloalkoxy, C₃₋₆ alkenyloxy, C₃₋₆ alkinyloxy, C₁₋₃ alkoxy(C₁₋₃)alkoxy, benzyloxy (wherein phenyl is optionally substituted by halogen, C₁₋₄ alkyl, C₁₋₄ alkoxy, C₁₋₄ haloalkyl, C₁₋₄ haloalkoxy, cyano or nitro), C₁₋₆ alkylthio, C₁₋₆ haloalkylthio, formyl, C₁₋₆ alkylcarbonyl, phenylcarbonyl (where phenyl is optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, R⁸⁰R⁸¹N—C(O)— or R⁸⁰R⁸¹N—C(S)—, wherein R⁸⁰ and R⁸¹ are independently hydrogen, C₁₋₆ alkyl, C₃₋₆alkenyl C₃₋₆ alkinyl or phenyl (which is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl); or the groups R⁷⁴ and R⁷⁵ together with the carbon atom to which they are attached form a three to six membered ring, containing at least 2 carbon atoms and optionally containing a sulphur atom, an oxygen atom, S(O), S(O)₂, C(O) or a group NR⁸², wherein R⁸² is hydrogen, C₁₋₆ alkyl, C₂₋₆-alkenyl, C₃₋₆-alkinyl or benzyl (where phenyl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl); the ring being optionally substituted by one or two C₁₋₃ alkyl groups;

Even more preferably L is a direct bond or CH₂. Most preferably L is a direct bond.

Preferably R⁴⁶, R⁴⁷, R⁴⁸ and R⁴⁹ are independently hydrogen, fluoro, C₁₋₆ alkyl, C₁₋₆ haloalkyl, hydroxy(C₁₋₆)alkyl, C₁₋₆ alkoxy(C₁₋₆)alkyl, phenyl(C₁₋₃)alkyl (wherein phenyl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl, C₁₋₆ alkoxycarbonyl), C₁₋₃ cycloalkyl(C₁₋₃)alkyl, C₃₋₅ cycloalkyl, 1,3-dioxolan-2-yl, phenyl (which may be optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl), C₁₋₆ alkylcarbonyl, phenylcarbonyl (where phenyl is optionally substituted by halogen, C₁₋₄ alkyl, C₁₋₄ alkoxy, C₁₋₄ haloalkyl, C₁₋₄ haloalkoxy, cyano or nitro), C₁₋₆ alkoxycarbonyl, C₁₋₆ alkylthiocarbonyl, R⁸³R⁸⁴N—C(O)— or R⁸³R⁸⁴N—C(S)—, wherein R⁸³ and R⁸⁴ are independently hydrogen, C₁₋₆ alkyl, C₃₋₆ alkenyl C₃₋₆ alkinyl or phenyl (which may be optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl); or the groups R⁴⁶ and R⁴⁷ and/or R⁴⁸ and R⁴⁹ together with the carbon atom to which they are attached form a three to six membered ring, containing at least 2 carbon atoms and optionally containing one or two sulphur and/or one or two non-adjacent oxygen atoms or a group S(O), S(O)₂, C(O) or NR⁸⁵, wherein R⁸⁵ is hydrogen, C₁₋₆-alkyl, C₂₋₆-alkenyl, C₃₋₆-alkinyl or benzyl (which is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl); the ring being optionally substituted by one or two C₁₋₃-alkyl groups; or two of the groups R⁴⁶, R⁴⁷, R⁴⁸ and R⁴⁹ attached to different atoms together with the atoms to which they are attached form a three to six membered ring, that optionally contains one or two sulphur and/or one or two non-adjacent oxygen atoms or a group S(O), S(O)₂, C(O) or NR⁸⁵, the ring being optionally substituted by one or two C₁₋₃-alkyl groups.

More preferably R⁴⁶, R⁴⁷, R⁴⁸ and R⁴⁹ are independently hydrogen, halogen, C₁₋₆ alkyl, C₁₋₆ haloalkyl, hydroxyl(C₁₋₆)alkyl, C₁₋₆ alkoxy(C₁₋₆)alkyl, phenyl(C₁₋₂)alkyl (wherein phenyl is optionally substituted by halogen, C₁₋₄ alkyl, C₁₋₄ alkoxy, C₁₋₄ haloalkyl, C₁₋₄ haloalkoxy, cyano, C₁₋₆ alkylsulfonyl, C₁₋₆ alkoxycarbonyl), C₃₋₆ cycloalkyl(C₁₋₃)alkyl, C₃₋₅ cycloalkyl, phenyl (which is substituted by halogen, C₁₋₄ alkyl, C₁₋₄ alkoxy, C₁₋₄ haloalkyl, C₁₋₄ haloalkoxy, cyano, C₁₋₆ alkylsulfonyl or C₁₋₆ alkoxycarbonyl), C₁₋₆ alkylcarbonyl, phenylcarbonyl (where phenyl is optionally substituted by halogen, C₁₋₄ alkyl, C₁₋₄ alkoxy, C₁₋₄ haloalkyl or C₁₋₄ haloalkoxy), C₁₋₆ alkoxycarbonyl, C₁₋₆ alkylthiocarbonyl, R⁸³R⁸⁴N—C(O)— or R⁸³R⁸⁴N—C(S)—, wherein R⁸³ and R⁸⁴ are independently hydrogen, C₁₋₆ alkyl, C₃₋₆ alkenyl, C₃₋₆ alkinyl or phenyl (which is optionally substituted by halogen, C₁₋₄ alkyl, C₁₋₄ alkoxy, C₁₋₄ haloalkyl, C₁₋₆ alkylsulfonyl or C₁₋₆ alkoxycarbonyl); or the groups R⁴⁶ and R⁴⁷ and/or R⁴⁸ and R⁴⁹ together with the carbon atom to which they are attached form a three to six membered ring, the ring being optionally substituted by one or two C₁₋₃ alkyl groups; or two of the groups R⁴⁶, R⁴⁷, R⁴⁴ and R⁴⁹ attached to different atoms together with the atoms to which they are attached form a three to five membered ring, the ring being optionally substituted by one or two C₁₋₃ alkyl groups.

Most preferably R⁴⁶, R⁴⁷, R⁴⁸ and R⁴⁹ are hydrogen.

It is preferred that the ring

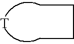

is a 6-membered aromatic ring or is a 5 or 6 membered heteroaromatic ring, wherein the ring members are each independently CH, S, N, NR⁴, O or CR⁴, wherein R⁴ is as defined herein, provided that there are no more than one O or S atoms present in the ring. In a particularly preferred group of compounds T is a 6-membered aromatic ring, n is 1, 2, or 3 and at least one substituent R⁴ is selected from fluoro, methyl, fluoromethyl, difluoromethyl, and trifluoromethyl.

More preferably the ring

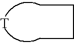

is a benzene, thiophene, furan, pyridine, pyrimidine, pyrazine, pyridazine, triazine, pyrrole, imidazole, pyrazole, oxazole, thiazole, isoxazole, isothiazole, [1,2,3]triazole, [1,2,3]oxadiazole or [1,2,3]thiadiazole ring.

Most preferably the ring

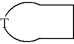

is a benzene, pyridine or thiophene ring, especially a benzene ring.

Preferably T and Y complete an indane ring system. A particularly preferred group of compounds wherein T and Y form an indane ring system are those wherein at least one of R¹⁰ and R⁷² is hydrogen.

Another especially preferred group of compounds are those compounds of formula (IA)

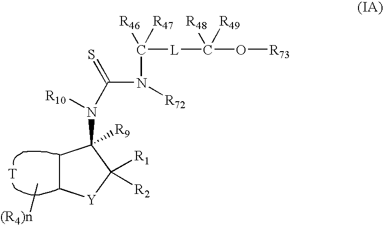

where the chirality on the R⁹ bearing carbon atom is that shown in the structure above, and L, T, Y, R¹, R², R⁴, R⁹, R¹⁰, R⁴⁶, R⁴⁷, R⁴⁸, R⁴⁹, R⁷², R⁷³ and n are as defined in relation to formula (I).

Another preferred group of compounds are those of formula I wherein T is phenyl, Y is CR⁵R⁶ or CR⁵R⁶CR⁷R⁸, L is a direct bond, each of R⁴⁶, R⁴⁷, R⁴⁸, and R⁴⁹ are hydrogen, and R¹, R², R⁴, R⁵, R⁶, R⁷, R⁸, R⁹, R¹⁰, R⁷² and R⁷³ are as defined herein.

Another preferred group of compounds of formula I are those wherein Y is O, S(O)ₘ, NR³, SO₂—NR³, NR³SO₂, NR³—O, O—NR³, O—CR⁷R⁸, S(O)ₘ—CR⁷R⁸, NR³—CR⁷R⁸, CR⁵R⁶—O, CR⁵R⁶—S(O)ₘ or CR⁵R⁶—NR³ wherein R³, R⁵, R⁶, R⁷ and R⁸ are as defined herein, and wherein L, T, R⁴, R⁹, R¹⁰, R⁴⁶, R⁴⁷, R⁴⁸, R⁴⁹, R⁷² and R⁷³ are as defined herein.

Another preferred group of compounds are those of formula I wherein T is a benzene ring, Y is CH₂ or CH₂CH₂, L is a direct bond, and R¹, R², R⁹, R⁴⁶, R⁴⁷, R⁴⁸ and R⁴⁹ are each hydrogen.

Another preferred group of compounds are those of formula I wherein at least one of R¹, R², R⁵, R⁶, R⁷, R⁸ or R⁹ is other than hydrogen.

In highly preferred compounds of formula I, L is a direct bond, R⁴⁶, R⁴⁷, R⁴⁸, R⁴⁹ are hydrogen, and least one of R¹⁰ and R⁷² is hydrogen.

The compounds in Tables 1 to 11 below illustrate the compounds of the invention.

TABLE 1

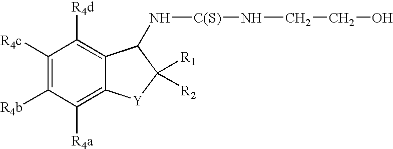

| Cpd. No | R₁ | R₂ | Y | R₄a | R₄b | R₄c | R₄d | Mp [° C.] |
|---|---|---|---|---|---|---|---|---|
| 1.001 | H | H | CH₂ | H | H | H | H | 119-121 |
| 1.002 | H | H | CH₂ | H | H | Ph | H | — |
| 1.003 | H | H | CH₂ | H | H | SCH₃ | H | — |
| 1.004 | H | H | CH₂ | H | H | S(O)CH₃ | H | — |
| 1.005 | H | H | CH₂ | H | H | SO₂CH₃ | H | — |
| 1.006 | H | H | CH₂ | H | H | SCF₃ | H | — |
| 1.007 | H | H | CH₂ | H | H | SCHF₂ | H | — |
| 1.008 | H | H | CH₂ | H | H | SOCHF₂ | H | — |
| 1.009 | H | H | CH₂ | H | H | OCHF₂ | H | — |
| 1.010 | H | H | CH₂ | H | H | OCF₃ | H | — |
| 1.011 | H | H | CH₂ | H | H | OCH₃ | H | — |
| 1.012 | H | H | CH₂ | H | H | F | H | 129-131 |
| 1.013 | H | H | CH₂ | H | H | Cl | H | — |
| 1.014 | H | H | CH₂ | H | H | Br | H | — |
| 1.015 | H | H | CH₂ | H | H | I | H | — |
| 1.016 | H | H | CH₂ | H | H | CH₂F | H | — |
| 1.017 | H | H | CH₂ | H | H | CHF₂ | H | — |
| 1.018 | H | H | CH₂ | H | H | CF₃ | H | — |
| 1.019 | H | H | CH₂ | H | H | CCl₃ | H | — |
| 1.020 | H | H | CH₂ | H | H | NO₂ | H | solid |
| 1.021 | H | H | CH₂ | H | H | CHO | H | — |
| 1.022 | H | H | CH₂ | H | H | CH₂OH | H | — |
| 1.023 | H | H | CH₂ | H | H | OH | H | — |
| 1.024 | H | H | CH₂ | H | H | NH₂ | H | — |
| 1.025 | H | H | CH₂ | H | H | NHCOCH₃ | H | — |
| 1.026 | H | H | CH₂ | H | H | OSO₂CF₃ | H | — |
| 1.027 | H | H | CH₂ | H | H | CH₃ | H | 142-144 |
| 1.028 | H | H | CH₂ | H | H | CO₂H | H | — |
| 1.029 | H | H | CH₂ | H | H | CO₂CH₃ | H | — |
| 1.030 | H | H | CH₂ | H | H | CN | H | — |
| 1.031 | H | H | CH₂ | H | H | CH═CH₂ | H | — |
| 1.032 | H | H | CH₂ | H | H | CCH | H | — |
| 1.033 | H | H | CH₂ | H | H | CONH₂ | H | — |
| 1.034 | H | H | CH₂ | H | H | N(CH₃)₂ | H | — |
| 1.035 | H | H | CH₂ | H | H | CH₂—CH═CH₂ | H | — |
| 1.036 | H | H | CH₂ | H | H | COCH₃ | H | — |
| 1.037 | H | H | CH₂ | H | H | CF₂CH₃ | H | — |
| 1.038 | H | H | CH₂ | H | H | CHFCH₃ | H | — |
| 1.039 | H | H | CH₂ | CH₂F | H | H | H | — |
| 1.040 | H | H | CH₂ | CHF₂ | H | H | H | — |
| 1.041 | H | H | CH₂ | CF₃ | H | H | H | — |
| 1.042 | H | H | CH₂ | CCl₃ | H | H | H | — |
| 1.043 | H | H | CH₂ | NO₂ | H | H | H | — |
| 1.044 | H | H | CH₂ | CHO | H | H | H | — |
| 1.045 | H | H | CH₂ | CH₂OH | H | H | H | — |
| 1.046 | H | H | CH₂ | OH | H | H | H | — |
| 1.047 | H | H | CH₂ | NH₂ | H | H | H | — |
| 1.048 | H | H | CH₂ | NHCOCH₃ | H | H | H | — |
| 1.049 | H | H | CH₂ | OSO₂CF₃ | H | H | H | — |
| 1.050 | H | H | CH₂ | CH₃ | H | H | H | 90-93 |
| 1.051 | H | H | CH₂ | CO₂H | H | H | H | — |
| 1.052 | H | H | CH₂ | CO₂CH₃ | H | H | H | — |
| 1.053 | H | H | CH₂ | CN | H | H | H | — |
| 1.054 | H | H | CH₂ | CH═CH₂ | H | H | H | — |
| 1.055 | H | H | CH₂ | CCH | H | H | H | — |
| 1.056 | H | H | CH₂ | CONH₂ | H | H | H | — |
| 1.057 | H | H | CH₂ | N(CH₃)₂ | H | H | H | — |
| 1.058 | H | H | CH₂ | CH₂—CH═CH₂ | H | H | H | — |
| 1.059 | H | H | CH₂ | COCH₃ | H | H | H | — |
| 1.060 | H | H | CH₂ | CF₂CH₃ | H | H | H | — |
| 1.061 | H | H | CH₂ | CHFCH₃ | H | H | H | — |
| 1.062 | H | H | CH₂ | OPh | H | H | H | — |
| 1.063 | H | H | CH₂ | Ph | H | H | H | — |
| 1.064 | H | H | CH₂ | SCH₃ | H | H | H | — |
| 1.065 | H | H | CH₂ | S(O)CH₃ | H | H | H | — |
| 1.066 | H | H | CH₂ | SO₂CH₃ | H | H | H | — |
| 1.067 | H | H | CH₂ | SCF₃ | H | H | H | — |
| 1.068 | H | H | CH₂ | SCHF₂ | H | H | H | 103-105 |
| 1.069 | H | H | CH₂ | S(O)CHF₂ | H | H | H | — |
| 1.070 | H | H | CH₂ | OCHF₂ | H | H | H | — |
| 1.071 | H | H | CH₂ | OCF₃ | H | H | H | — |
| 1.072 | H | H | CH₂ | OCH₃ | H | H | H | — |
| 1.073 | H | H | CH₂ | F | H | H | H | 123-125 |
| 1.074 | H | H | CH₂ | Cl | H | H | H | — |
| 1.075 | H | H | CH₂ | Br | H | H | H | — |
| 1.076 | H | H | CH₂ | I | H | H | H | — |
| 1.077 | H | H | CH₂ | CH₂F | H | F | H | — |
| 1.078 | H | H | CH₂ | CHF₂ | H | F | H | — |
| 1.079 | H | H | CH₂ | CF₃ | H | F | H | — |
| 1.080 | H | H | CH₂ | CCl₃ | H | F | H | — |
| 1.081 | H | H | CH₂ | NO₂ | H | F | H | — |
| 1.082 | H | H | CH₂ | SCF₃ | H | F | H | — |
| 1.083 | H | H | CH₂ | SCHF₂ | H | F | H | — |
| 1.084 | H | H | CH₂ | S(O)CHF₂ | H | F | H | — |
| 1.085 | H | H | CH₂ | OCHF₂ | H | F | H | — |
| 1.086 | H | H | CH₂ | OCF₃ | H | F | H | — |
| 1.087 | H | H | CH₂ | OCH₃ | H | F | H | — |
| 1.088 | H | H | CH₂ | F | H | F | H | 124-125 |
| 1.089 | H | H | CH₂ | Cl | H | F | H | — |
| 1.090 | H | H | CH₂ | Br | H | F | H | — |
| 1.091 | H | H | CH₂ | I | H | F | H | — |
| 1.092 | H | H | CH₂ | CH₃ | H | F | H | 121-123 |
| 1.093 | H | H | CH₂ | OH | H | F | H | — |
| 1.094 | H | H | CH₂ | F | H | Ph | H | — |
| 1.095 | H | H | CH₂ | F | H | SCH₃ | H | — |
| 1.096 | H | H | CH₂ | F | H | S(O)CH₃ | H | — |
| 1.097 | H | H | CH₂ | F | H | SO₂CH₃ | H | — |
| 1.098 | H | H | CH₂ | F | H | SCF₃ | H | — |
| 1.099 | H | H | CH₂ | F | H | SCHF₂ | H | — |
| 1.100 | H | H | CH₂ | F | H | S(O)CHF₂ | H | — |
| 1.101 | H | H | CH₂ | F | H | OCHF₂ | H | — |
| 1.102 | H | H | CH₂ | F | H | OCF₃ | H | — |
| 1.103 | H | H | CH₂ | F | H | OCH₃ | H | — |
| 1.104 | H | H | CH₂ | F | H | Cl | H | — |
| 1.105 | H | H | CH₂ | F | H | Br | H | — |
| 1.106 | H | H | CH₂ | F | H | I | H | — |
| 1.107 | H | H | CH₂ | F | H | CH₂F | H | — |
| 1.108 | H | H | CH₂ | F | H | CHF₂ | H | — |
| 1.109 | H | H | CH₂ | F | H | CF₃ | H | — |
| 1.110 | H | H | CH₂ | F | H | CCl₃ | H | — |
| 1.111 | H | H | CH₂ | F | H | NO₂ | H | — |
| 1.112 | H | H | CH₂ | F | H | CHO | H | — |
| 1.113 | H | H | CH₂ | F | H | CH₂OH | H | — |
| 1.114 | H | H | CH₂ | F | H | OH | H | — |
| 1.115 | H | H | CH₂ | F | H | NH₂ | H | — |
| 1.116 | H | H | CH₂ | F | H | NHCOCH₃ | H | — |
| 1.117 | H | H | CH₂ | F | H | OSO₂CF₃ | H | — |
| 1.118 | H | H | CH₂ | F | H | CH₃ | H | resin |
| 1.119 | H | H | CH₂ | F | H | CO₂H | H | — |
| 1.120 | H | H | CH₂ | F | H | CO₂CH₃ | H | — |
| 1.121 | H | H | CH₂ | F | H | CN | H | — |
| 1.122 | H | H | CH₂ | F | H | CH═CH₂ | H | — |
| 1.123 | H | H | CH₂ | F | H | CCH | H | — |
| 1.124 | H | H | CH₂ | F | H | CONH₂ | H | — |
| 1.125 | H | H | CH₂ | F | H | N(CH₃)₂ | H | — |
| 1.126 | H | H | CH₂ | F | H | CH₂—CH═CH₂ | H | — |
| 1.127 | H | H | CH₂ | F | H | COCH₃ | H | — |
| 1.128 | H | H | CH₂ | F | H | CF₂CH₃ | H | — |
| 1.129 | CH₃ | H | CH₂ | F | H | H | H | — |
| 1.130 | CH₃ | H | CH₂ | Cl | H | H | H | — |
| 1.131 | CH₃ | H | CH₂ | Br | H | H | H | — |
| 1.132 | CH₃ | H | CH₂ | I | H | H | H | — |
| 1.133 | CH₃ | H | CH₂ | CH₃ | H | H | H | — |
| 1.134 | CH₃ | H | CH₂ | H | H | H | H | — |
| 1.135 | CH₃ | H | CH₂ | CH₂F | H | H | H | — |
| 1.136 | CH₃ | H | CH₂ | CHF₂ | H | H | H | — |
| 1.137 | CH₃ | H | CH₂ | CF₃ | H | H | H | — |
| 1.138 | CH₃ | H | CH₂ | NO₂ | H | H | H | — |
| 1.139 | CH₃ | H | CH₂ | OCHF₂ | H | H | H | — |
| 1.140 | CH₃ | H | CH₂ | OCF₃ | H | H | H | — |
| 1.141 | CH₃ | H | CH₂ | F | H | F | H | — |
| 1.142 | CH₃ | H | CH₂ | Cl | H | F | H | — |
| 1.143 | CH₃ | H | CH₂ | Br | H | F | H | — |
| 1.144 | CH₃ | H | CH₂ | I | H | F | H | — |
| 1.145 | CH₃ | H | CH₂ | CH₃ | H | F | H | — |
| 1.146 | CH₃ | H | CH₂ | H | H | F | H | — |
| 1.147 | CH₃ | H | CH₂ | CH₂F | H | F | H | — |
| 1.148 | CH₃ | H | CH₂ | CHF₂ | H | F | H | — |
| 1.149 | CH₃ | H | CH₂ | CF₃ | H | F | H | — |
| 1.150 | CH₃ | H | CH₂ | NO₂ | H | F | H | — |
| 1.151 | CH₃ | H | CH₂ | OCHF₂ | H | F | H | — |
| 1.152 | CH₃ | H | CH₂ | OCF₃ | H | F | H | — |
| 1.153 | F | H | CH₂ | F | H | H | H | — |
| 1.154 | F | H | CH₂ | Cl | H | H | H | — |
| 1.155 | F | H | CH₂ | Br | H | H | H | — |
| 1.156 | F | H | CH₂ | I | H | H | H | — |
| 1.157 | F | H | CH₂ | CH₃ | H | H | H | — |
| 1.158 | F | H | CH₂ | H | H | H | H | — |
| 1.159 | F | H | CH₂ | CH₂F | H | H | H | — |
| 1.160 | F | H | CH₂ | CHF₂ | H | H | H | — |
| 1.161 | F | H | CH₂ | CF₃ | H | H | H | — |
| 1.162 | F | H | CH₂ | NO₂ | H | H | H | — |
| 1.163 | F | H | CH₂ | OCHF₂ | H | H | H | — |
| 1.164 | F | H | CH₂ | OCF₃ | H | H | H | — |
| 1.165 | F | H | CH₂ | F | H | F | H | — |
| 1.166 | F | H | CH₂ | Cl | H | F | H | — |
| 1.167 | F | H | CH₂ | Br | H | F | H | — |
| 1.168 | F | H | CH₂ | I | H | F | H | — |
| 1.169 | F | H | CH₂ | CH₃ | H | F | H | — |
| 1.170 | F | H | CH₂ | H | H | F | H | — |
| 1.171 | F | H | CH₂ | CH₂F | H | F | H | — |
| 1.172 | F | H | CH₂ | CHF₂ | H | F | H | — |
| 1.173 | F | H | CH₂ | CF₃ | H | F | H | — |
| 1.174 | F | H | CH₂ | NO₂ | H | F | H | — |
| 1.175 | F | H | CH₂ | OCHF₂ | H | F | H | — |
| 1.176 | F | H | CH₂ | OCF₃ | H | F | H | — |
| 1.177 | H | H | CH₂ | H | H | H | F | — |
| 1.178 | H | H | CH₂ | H | H | H | Cl | — |
| 1.179 | H | H | CH₂ | H | H | H | Br | — |
| 1.180 | H | H | CH₂ | H | H | H | I | — |
| 1.181 | H | H | CH₂ | H | H | H | CH₃ | — |
| 1.182 | H | H | CH₂ | H | H | H | CH₂F | — |
| 1.183 | H | H | CH₂ | H | H | H | CHF₂ | — |
| 1.184 | H | H | CH₂ | H | H | H | CF₃ | — |
| 1.185 | H | H | CH₂ | H | H | H | NO₂ | — |
| 1.186 | H | H | CH₂ | H | H | H | OCHF₂ | — |
| 1.187 | H | H | CH₂ | H | H | H | OCF₃ | — |
| 1.188 | H | H | CH₂ | H | H | H | Ph | — |
| 1.189 | H | H | CH₂ | H | H | H | NH₂ | — |
| 1.190 | H | H | CH₂ | H | H | H | NHCOCH₃ | — |
| 1.191 | H | H | CH₂ | Cl | H | H | F | — |
| 1.192 | H | H | CH₂ | Cl | H | H | Cl | — |
| 1.193 | H | H | CH₂ | Cl | H | H | Br | — |
| 1.194 | H | H | CH₂ | Cl | H | H | I | — |
| 1.195 | H | H | CH₂ | Cl | H | H | CH₃ | — |
| 1.196 | H | H | CH₂ | Cl | H | H | CH₂F | — |
| 1.197 | H | H | CH₂ | Cl | H | H | CHF₂ | — |
| 1.198 | H | H | CH₂ | Cl | H | H | CF₃ | — |
| 1.199 | H | H | CH₂ | Cl | H | H | NO₂ | — |
| 1.200 | H | H | CH₂ | Cl | H | H | OCHF₂ | — |
| 1.201 | H | H | CH₂ | Cl | H | H | OCF₃ | — |
| 1.202 | H | H | CH₂ | Cl | H | H | Ph | — |
| 1.203 | H | H | CH₂ | Cl | H | H | NH₂ | — |
| 1.204 | H | H | CH₂ | Cl | H | H | NHCOCH₃ | — |
| 1.205 | H | H | CH₂ | Cl | H | Cl | H | 134-136 |
| 1.206 | H | H | CH₂ | Cl | H | Br | H | — |
| 1.207 | H | H | CH₂ | Cl | H | I | H | — |
| 1.208 | H | H | CH₂ | Cl | H | CH₃ | H | — |
| 1.209 | H | H | CH₂ | Cl | H | CH₂F | H | — |
| 1.210 | H | H | CH₂ | Cl | H | CHF₂ | H | — |
| 1.211 | H | H | CH₂ | Cl | H | CF₃ | H | — |
| 1.212 | H | H | CH₂ | Cl | H | NO₂ | H | — |
| 1.213 | H | H | CH₂ | Cl | H | OCHF2 | H | — |
| 1.214 | H | H | CH₂ | Cl | H | OCF₃ | H | — |
| 1.215 | H | H | CH₂ | Cl | H | Ph | H | — |
| 1.216 | H | H | CH₂ | Cl | H | NH₂ | H | — |
| 1.217 | H | H | CH₂ | Cl | H | NHCOCH₃ | H | — |
| 1.218 | H | H | C(═CH₂) | F | H | F | H | — |
| 1.219 | H | H | C(═CH₂) | Cl | H | F | H | — |
| 1.220 | H | H | C(═CH₂) | Br | H | F | H | — |
| 1.221 | H | H | C(═CH₂) | I | H | F | H | — |
| 1.222 | H | H | C(═CH₂) | CH₃ | H | F | H | — |
| 1.223 | H | H | C(═CH₂) | CH₂F | H | F | H | — |
| 1.224 | H | H | C(═CH₂) | CHF₂ | H | F | H | — |
| 1.225 | H | H | C(═CH₂) | CF₃ | H | F | H | — |
| 1.226 | H | H | C(═CH₂) | NO₂ | H | F | H | — |
| 1.227 | H | H | C(═CH₂) | OCHF₂ | H | F | H | — |
| 1.228 | H | H | C(═CH₂) | OCF₃ | H | F | H | — |
| 1.229 | H | H | C(═CH₂) | NHCOCH₃ | H | F | H | — |
| 1.230 | H | H | C(═CH₂) | F | H | Cl | H | — |
| 1.231 | H | H | C(═CH₂) | F | H | Br | H | — |
| 1.232 | H | H | C(═CH₂) | F | H | I | H | — |
| 1.233 | H | H | C(═CH₂) | F | H | CH₃ | H | — |
| 1.234 | H | H | C(═CH₂) | F | H | CH₂F | H | — |
| 1.235 | H | H | C(═CH₂) | F | H | CHF₂ | H | — |
| 1.236 | H | H | C(═CH₂) | F | H | CF₃ | H | — |
| 1.237 | H | H | C(═CH₂) | F | H | NO₂ | H | — |
| 1.238 | H | H | C(═CH₂) | F | H | OCHF₂ | H | — |
| 1.239 | H | H | C(═CH₂) | F | H | OCF₃ | H | — |
| 1.240 | H | H | C(═CH₂) | F | H | H | H | — |
| 1.241 | H | H | CHF | F | H | F | H | — |
| 1.242 | H | H | CHF | Cl | H | F | H | — |
| 1.243 | H | H | CHF | Br | H | F | H | — |
| 1.244 | H | H | CHF | I | H | F | H | — |
| 1.245 | H | H | CHF | CH₃ | H | F | H | — |
| 1.246 | H | H | CHF | CH₂F | H | F | H | — |
| 1.247 | H | H | CHF | CHF₂ | H | F | H | — |
| 1.248 | H | H | CHF | CF₃ | H | F | H | — |
| 1.249 | H | H | CHF | NO₂ | H | F | H | — |
| 1.250 | H | H | CHF | OCHF₂ | H | F | H | — |
| 1.251 | H | H | CHF | OCF₃ | H | F | H | — |
| 1.252 | H | H | CHF | NHCOCH₃ | H | F | H | — |
| 1.253 | H | H | CHF | F | H | Cl | H | — |
| 1.254 | H | H | CHF | F | H | Br | H | — |
| 1.255 | H | H | CHF | F | H | I | H | — |
| 1.256 | H | H | CHF | F | H | CH₃ | H | — |
| 1.257 | H | H | CHF | F | H | CH₂F | H | — |
| 1.258 | H | H | CHF | F | H | CHF₂ | H | — |
| 1.259 | H | H | CHF | F | H | CF₃ | H | — |
| 1.260 | H | H | CHF | F | H | NO₂ | H | — |
| 1.261 | H | H | CHF | F | H | OCHF₂ | H | — |
| 1.262 | H | H | CHF | F | H | OCF₃ | H | — |
| 1.263 | H | H | CHF | F | H | H | H | — |
| 1.264 | H | H | CH(CH₃) | F | H | F | H | — |
| 1.265 | H | H | CH(CH₃) | Cl | H | F | H | — |
| 1.266 | H | H | CH(CH₃) | Br | H | F | H | — |
| 1.267 | H | H | CH(CH₃) | I | H | F | H | — |
| 1.268 | H | H | CH(CH₃) | CH₃ | H | F | H | — |
| 1.269 | H | H | CH(CH₃) | CH₂F | H | F | H | — |
| 1.270 | H | H | CH(CH₃) | CHF₂ | H | F | H | — |
| 1.271 | H | H | CH(CH₃) | CF₃ | H | F | H | — |
| 1.272 | H | H | CH(CH₃) | NO₂ | H | F | H | — |
| 1.273 | H | H | CH(CH₃) | OCHF₂ | H | F | H | — |
| 1.274 | H | H | CH(CH₃) | OCF₃ | H | F | H | — |
| 1.275 | H | H | CH(CH₃) | NHCOCH₃ | H | F | H | — |
| 1.276 | H | H | CH(CH₃) | F | H | Cl | H | — |
| 1.277 | H | H | CH(CH₃) | F | H | Br | H | — |
| 1.278 | H | H | CH(CH₃) | F | H | I | H | — |
| 1.279 | H | H | CH(CH₃) | F | H | CH₃ | H | — |
| 1.280 | H | H | CH(CH₃) | F | H | CH₂F | H | — |
| 1.281 | H | H | CH(CH₃) | F | H | CHF₂ | H | — |
| 1.282 | H | H | CH(CH₃) | F | H | CF₃ | H | — |
| 1.283 | H | H | CH(CH₃) | F | H | NO₂ | H | — |
| 1.284 | H | H | CH(CH₃) | F | H | OCHF₂ | H | — |
| 1.285 | H | H | CH(CH₃) | F | H | OCF₃ | H | — |
| 1.286 | H | H | CH(CH₃) | F | H | H | H | — |
| 1.287 | OH | H | CH₂ | F | H | F | H | — |
| 1.288 | OH | H | CH₂ | Cl | H | F | H | — |
| 1.289 | OH | H | CH₂ | Br | H | F | H | — |
| 1.290 | OH | H | CH₂ | I | H | F | H | — |
| 1.291 | OH | H | CH₂ | CH₃ | H | F | H | — |
| 1.292 | OH | H | CH₂ | CH₂F | H | F | H | — |
| 1.293 | OH | H | CH₂ | CHF₂ | H | F | H | — |
| 1.294 | OH | H | CH₂ | CF₃ | H | F | H | — |
| 1.295 | OH | H | CH₂ | NO₂ | H | F | H | — |
| 1.296 | OH | H | CH₂ | OCHF₂ | H | F | H | — |
| 1.297 | OH | H | CH₂ | OCF₃ | H | F | H | — |
| 1.298 | OH | H | CH₂ | NHCOCH₃ | H | F | H | — |
| 1.299 | OH | H | CH₂ | F | H | Cl | H | — |
| 1.300 | OH | H | CH₂ | F | H | Br | H | — |
| 1.301 | OH | H | CH₂ | F | H | I | H | — |
| 1.302 | OH | H | CH₂ | F | H | CH₃ | H | — |
| 1.303 | OH | H | CH₂ | F | H | CH₂F | H | — |
| 1.304 | OH | H | CH₂ | F | H | CHF₂ | H | — |
| 1.305 | OH | H | CH₂ | F | H | CF₃ | H | — |
| 1.306 | OH | H | CH₂ | F | H | NO₂ | H | — |
| 1.307 | OH | H | CH₂ | F | H | OCHF₂ | H | — |
| 1.308 | OH | H | CH₂ | F | H | OCF₃ | H | — |
| 1.309 | OH | H | CH₂ | F | H | H | H | — |
| 1.310 | F | F | CH₂ | F | H | Cl | H | — |
| 1.311 | F | F | CH₂ | F | H | Br | H | — |
| 1.312 | F | F | CH₂ | F | H | I | H | — |
| 1.313 | F | F | CH₂ | F | H | CH₃ | H | — |
| 1.314 | F | F | CH₂ | F | H | CH₂F | H | — |
| 1.315 | F | F | CH₂ | F | H | CHF₂ | H | — |
| 1.316 | F | F | CH₂ | F | H | CF₃ | H | — |
| 1.317 | F | F | CH₂ | F | H | NO₂ | H | — |
| 1.318 | F | F | CH₂ | F | H | OCHF₂ | H | — |
| 1.319 | F | F | CH₂ | F | H | OCF₃ | H | — |
| 1.320 | F | F | CH₂ | F | H | H | H | — |
| 1.321 | F | F | CH₂ | F | H | F | H | — |
| 1.322 | OCH₃ | H | CH₂ | F | H | Cl | H | — |
| 1.323 | OCH₃ | H | CH₂ | F | H | Br | H | — |
| 1.324 | OCH₃ | H | CH₂ | F | H | I | H | — |
| 1.325 | OCH₃ | H | CH₂ | F | H | CH₃ | H | — |
| 1.326 | OCH₃ | H | CH₂ | F | H | CH₂F | H | — |
| 1.327 | OCH₃ | H | CH₂ | F | H | CHF₂ | H | — |
| 1.328 | OCH₃ | H | CH₂ | F | H | CF₃ | H | — |
| 1.329 | OCH₃ | H | CH₂ | F | H | NO₂ | H | — |
| 1.330 | OCH₃ | H | CH₂ | F | H | OCHF₂ | H | — |
| 1.331 | OCH₃ | H | CH₂ | F | H | OCF₃ | H | — |
| 1.332 | OCH₃ | H | CH₂ | F | H | H | H | — |
| 1.333 | OCH₃ | H | CH₂ | F | H | F | H | — |
| 1.334 | H | H | CH₂ | H | F | H | H | — |
| 1.335 | H | H | CH₂ | H | Cl | H | H | — |
| 1.336 | H | H | CH₂ | F | F | H | H | — |
| 1.337 | H | H | CH₂ | H | CHF₂ | H | H | — |
| 1.338 | H | H | CH₂ | H | Br | H | H | — |
| 1.339 | H | H | CH₂ | H | CH₃ | H | H | — |
| 1.340 | H | H | CH₂ | H | I | H | H | — |
| 1.341 | H | H | CH₂—CH₂ | CH₂F | H | H | H | — |
| 1.342 | H | H | CH₂—CH₂ | CHF₂ | H | H | H | — |
| 1.343 | H | H | CH₂—CH₂ | CF₃ | H | H | H | — |
| 1.344 | H | H | CH₂—CH₂ | CCl₃ | H | H | H | — |
| 1.345 | H | H | CH₂—CH₂ | NO₂ | H | H | H | — |
| 1.346 | H | H | CH₂—CH₂ | CHO | H | H | H | — |
| 1.347 | H | H | CH₂—CH₂ | CH₂OH | H | H | H | — |
| 1.348 | H | H | CH₂—CH₂ | OH | H | H | H | — |
| 1.349 | H | H | CH₂—CH₂ | NH₂ | H | H | H | — |
| 1.350 | H | H | CH₂—CH₂ | NHCOCH₃ | H | H | H | — |
| 1.351 | H | H | CH₂—CH₂ | OSO₂CF₃ | H | H | H | — |
| 1.352 | H | H | CH₂—CH₂ | CH₃ | H | H | H | — |
| 1.353 | H | H | CH₂—CH₂ | CO₂H | H | H | H | — |
| 1.354 | H | H | CH₂—CH₂ | CO₂CH₃ | H | H | H | — |
| 1.355 | H | H | CH₂—CH₂ | CN | H | H | H | — |
| 1.356 | H | H | CH₂—CH₂ | CH═CH₂ | H | H | H | — |
| 1.357 | H | H | CH₂—CH₂ | CCH | H | H | H | — |
| 1.358 | H | H | CH₂—CH₂ | CONH₂ | H | H | H | — |
| 1.359 | H | H | CH₂—CH₂ | N(CH₃)₂ | H | H | H | — |
| 1.360 | H | H | CH₂—CH₂ | CH₂—CH═CH₂ | H | H | H | — |
| 1.361 | H | H | CH₂—CH₂ | COCH₃ | H | H | H | — |
| 1.362 | H | H | CH₂—CH₂ | CF₂CH₃ | H | H | H | — |
| 1.363 | H | H | CH₂—CH₂ | CHFCH₃ | H | H | H | — |
| 1.364 | H | H | CH₂—CH₂ | OPh | H | H | H | — |
| 1.365 | H | H | CH₂—CH₂ | Ph | H | H | H | — |
| 1.366 | H | H | CH₂—CH₂ | SCH₃ | H | H | H | — |
| 1.367 | H | H | CH₂—CH₂ | S(O)CH₃ | H | H | H | — |
| 1.368 | H | H | CH₂—CH₂ | SO₂CH₃ | H | H | H | — |
| 1.369 | H | H | CH₂—CH₂ | SCF₃ | H | H | H | — |
| 1.370 | H | H | CH₂—CH₂ | SCHF₂ | H | H | H | — |
| 1.371 | H | H | CH₂—CH₂ | S(O)CHF₂ | H | H | H | — |
| 1.372 | H | H | CH₂—CH₂ | OCHF₂ | H | H | H | — |
| 1.373 | H | H | CH₂—CH₂ | OCF₃ | H | H | H | — |
| 1.374 | H | H | CH₂—CH₂ | OCH₃ | H | H | H | — |
| 1.375 | H | H | CH₂—CH₂ | F | H | H | H | — |
| 1.376 | H | H | CH₂—CH₂ | Cl | H | H | H | — |
| 1.377 | H | H | CH₂—CH₂ | Br | H | H | H | — |
| 1.378 | H | H | CH₂—CH₂ | I | H | H | H | — |
| 1.379 | H | H | CH₂—CH₂ | CH₂F | H | F | H | — |
| 1.380 | H | H | CH₂—CH₂ | CHF₂ | H | F | H | — |
| 1.381 | H | H | CH₂—CH₂ | CF₃ | H | F | H | — |
| 1.382 | H | H | CH₂—CH₂ | CCl₃ | H | F | H | — |
| 1.383 | H | H | CH₂—CH₂ | NO₂ | H | F | H | — |
| 1.384 | H | H | CH₂—CH₂ | SCF₃ | H | F | H | — |
| 1.385 | H | H | CH₂—CH₂ | SCHF₂ | H | F | H | — |
| 1.386 | H | H | CH₂—CH₂ | S(O)CHF₂ | H | F | H | — |
| 1.387 | H | H | CH₂—CH₂ | OCHF₂ | H | F | H | — |
| 1.388 | H | H | CH₂—CH₂ | OCF₃ | H | F | H | — |
| 1.389 | H | H | CH₂—CH₂ | OCH₃ | H | F | H | — |
| 1.390 | H | H | CH₂—CH₂ | F | H | F | H | — |
| 1.391 | H | H | CH₂—CH₂ | Cl | H | F | H | — |
| 1.392 | H | H | CH₂—CH₂ | Br | H | F | H | — |
| 1.393 | H | H | CH₂—CH₂ | I | H | F | H | — |
| 1.394 | H | H | CH₂—CH₂ | CH₃ | H | F | H | — |
| 1.395 | H | H | CH₂—CH₂ | OH | H | F | H | — |
| 1.396 | H | H | CH₂—CH₂ | H | H | F | H | — |
| 1.397 | H | H | CH₂—CH₂ | H | H | Cl | H | — |
| 1.398 | H | H | CH₂—CH₂ | H | H | Br | H | — |
| 1.399 | H | H | CH₂—CH₂ | H | H | I | H | — |
| 1.400 | H | H | CH₂—CH₂ | H | H | CH₂F | H | — |
| 1.401 | H | H | CH₂—CH₂ | H | H | CHF₂ | H | — |
| 1.402 | H | H | CH₂—CH₂ | H | H | CF₃ | H | — |
| 1.403 | H | H | CH₂—CH₂ | H | H | CCl₃ | H | — |
| 1.404 | H | H | CH₂—CH₂ | H | H | NO₂ | H | — |
| 1.405 | H | H | CH₂—CH₂ | H | H | SCF₃ | H | — |
| 1.406 | H | H | CH₂—CH₂ | H | H | SCHF₂ | H | — |
| 1.407 | H | H | CH₂—CH₂ | H | H | S(O)CHF₂ | H | — |
| 1.408 | H | H | CH₂—CH₂ | H | H | OCHF₂ | H | — |
| 1.409 | H | H | CH₂—CH₂ | H | H | OCF₃ | H | — |
| 1.410 | H | H | O | F | H | H | H | — |
| 1.411 | H | H | O | Cl | H | H | H | — |
| 1.412 | H | H | O | Br | H | H | H | — |
| 1.413 | H | H | O | I | H | H | H | — |
| 1.414 | H | H | O | CH₃ | H | H | H | — |
| 1.415 | H | H | O | CH₂F | H | H | H | — |
| 1.416 | H | H | O | CHF₂ | H | H | H | — |
| 1.417 | H | H | O | CF₃ | H | H | H | — |
| 1.418 | H | H | O | F | H | F | H | — |
| 1.419 | H | H | O | OCHF₂ | H | H | H | — |
| 1.420 | H | H | O | OCF₃ | H | H | H | — |
| 1.421 | H | H | O | H | H | F | H | — |
| 1.422^{a)} | H | H | CH₂ | H | H | H | H | Resin |
| 1.423^{b)} | H | H | CH₂ | H | H | H | H | Resin |
| 1.424^{c)} | OH | H | CH₂ | H | H | H | H | 139-146 |
| 1.425^{a)} | H | H | CH₂ | F | H | H | H | Resin |
| 1.426^{b)} | H | H | CH₂ | F | H | H | H | Resin |

^{a)}S-Enantiomer.
^{b)}R-Enantiomer.
^{c)}Single diastereoisomer (anti).

TABLE 2

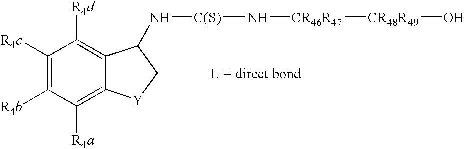

| Cpd. No | CR₄₆R₄₇ | CR₄₈R₄₉ | Y | R₄a | R₄b | R₄c | R₄d | Mp [° C.]/Ret ^{a)} |
|---|---|---|---|---|---|---|---|---|
| 2.001 | CH(CH₃) | CH₂ | CH₂ | CH₂F | H | H | H | — |
| 2.002 | CH(CH₃) | CH₂ | CH₂ | CHF₂ | H | H | H | — |
| 2.003 | CH(CH₃) | CH₂ | CH₂ | CF₃ | H | H | H | — |
| 2.004 | CH(CH₃) | CH₂ | CH₂ | CCl₃ | H | H | H | — |
| 2.005 | CH(CH₃) | CH₂ | CH₂ | NO₂ | H | H | H | — |
| 2.006 | CH(CH₃) | CH₂ | CH₂ | CHO | H | H | H | — |
| 2.007 | CH(CH₃) | CH₂ | CH₂ | CH₂OH | H | H | H | — |
| 2.008 | CH(CH₃) | CH₂ | CH₂ | OH | H | H | H | — |
| 2.009 | CH(CH₃) | CH₂ | CH₂ | NH₂ | H | H | H | — |
| 2.010 | CH(CH₃) | CH₂ | CH₂ | NHCOCH₃ | H | H | H | — |
| 2.011 | CH(CH₃) | CH₂ | CH₂ | OSO₂CF₃ | H | H | H | — |
| 2.012 | CH(CH₃) | CH₂ | CH₂ | CH₃ | H | H | H | Ret = 1.25 |
| 2.013 | CH(CH₃) | CH₂ | CH₂ | CO₂H | H | H | H | — |
| 2.014 | CH(CH₃) | CH₂ | CH₂ | CO₂CH₃ | H | H | H | — |
| 2.015 | CH(CH₃) | CH₂ | CH₂ | CN | H | H | H | — |
| 2.016 | CH(CH₃) | CH₂ | CH₂ | CH═CH₂ | H | H | H | — |
| 2.017 | CH(CH₃) | CH₂ | CH₂ | CCH | H | H | H | — |
| 2.018 | CH(CH₃) | CH₂ | CH₂ | CONH₂ | H | H | H | — |
| 2.019 | CH(CH₃) | CH₂ | CH₂ | N(CH₃)₂ | H | H | H | — |
| 2.020 | CH(CH₃) | CH₂ | CH₂ | CH₂—CH═CH₂ | H | H | H | — |
| 2.021 | CH(CH₃) | CH₂ | CH₂ | COCH₃ | H | H | H | — |
| 2.022 | CH(CH₃) | CH₂ | CH₂ | CF₂CH₃ | H | H | H | — |
| 2.023 | CH(CH₃) | CH₂ | CH₂ | CHFCH₃ | H | H | H | — |
| 2.024 | CH(CH₃) | CH₂ | CH₂ | OPh | H | H | H | — |
| 2.025 | CH(CH₃) | CH₂ | CH₂ | Ph | H | H | H | — |
| 2.026 | CH(CH₃) | CH₂ | CH₂ | SCH₃ | H | H | H | — |
| 2.027 | CH(CH₃) | CH₂ | CH₂ | S(O)CH₃ | H | H | H | — |
| 2.028 | CH(CH₃) | CH₂ | CH₂ | SO₂CH₃ | H | H | H | — |
| 2.029 | CH(CH₃) | CH₂ | CH₂ | SCF₃ | H | H | H | — |
| 2.030 | CH(CH₃) | CH₂ | CH₂ | SCHF₂ | H | H | H | — |
| 2.031 | CH(CH₃) | CH₂ | CH₂ | S(O)CHF₂ | H | H | H | — |
| 2.032 | CH(CH₃) | CH₂ | CH₂ | OCHF₂ | H | H | H | — |
| 2.033 | CH(CH₃) | CH₂ | CH₂ | OCF₃ | H | H | H | — |
| 2.034 | CH(CH₃) | CH₂ | CH₂ | OCH₃ | H | H | H | — |
| 2.035 | CH(CH₃) | CH₂ | CH₂ | F | H | H | H | Ret = 1.30 |
| 2.036 | CH(CH₃) | CH₂ | CH₂ | Cl | H | H | H | — |
| 2.037 | CH(CH₃) | CH₂ | CH₂ | Br | H | H | H | — |
| 2.038 | CH(CH₃) | CH₂ | CH₂ | I | H | H | H | — |
| 2.039 | CH(CH₃) | CH₂ | CH₂ | CH₂F | H | F | H | — |
| 2.040 | CH(CH₃) | CH₂ | CH₂ | CHF₂ | H | F | H | — |
| 2.041 | CH(CH₃) | CH₂ | CH₂ | CF₃ | H | F | H | — |
| 2.042 | CH(CH₃) | CH₂ | CH₂ | CCl₃ | H | F | H | — |
| 2.043 | CH(CH₃) | CH₂ | CH₂ | NO₂ | H | F | H | — |
| 2.044 | CH(CH₃) | CH₂ | CH₂ | SCF₃ | H | F | H | — |
| 2.045 | CH(CH₃) | CH₂ | CH₂ | SCHF₂ | H | F | H | — |
| 2.046 | CH(CH₃) | CH₂ | CH₂ | S(O)CHF₂ | H | F | H | — |
| 2.047 | CH(CH₃) | CH₂ | CH₂ | OCHF₂ | H | F | H | — |
| 2.048 | CH(CH₃) | CH₂ | CH₂ | OCF₃ | H | F | H | — |
| 2.049 | CH(CH₃) | CH₂ | CH₂ | OCH₃ | H | F | H | — |
| 2.050 | CH(CH₃) | CH₂ | CH₂ | F | H | F | H | Ret = 1.33 |
| 2.051 | CH(CH₃) | CH₂ | CH₂ | Cl | H | F | H | — |
| 2.052 | CH(CH₃) | CH₂ | CH₂ | Br | H | F | H | — |
| 2.053 | CH(CH₃) | CH₂ | CH₂ | I | H | F | H | — |
| 2.054 | CH(CH₃) | CH₂ | CH₂ | CH₃ | H | F | H | — |
| 2.055 | CH(CH₃) | CH₂ | CH₂ | OH | H | F | H | — |
| 2.056 | CH(CH₃) | CH₂ | CH₂ | H | H | H | F | — |
| 2.057 | CH(CH₃) | CH₂ | CH₂ | H | H | H | Cl | — |
| 2.058 | CH(CH₃) | CH₂ | CH₂ | H | H | H | Br | — |
| 2.059 | CH(CH₃) | CH₂ | CH₂ | H | H | H | I | — |
| 2.060 | CH(CH₃) | CH₂ | CH₂ | H | H | H | CH₃ | — |
| 2.061 | CH(CH₃) | CH₂ | CH₂ | H | H | H | CH₂F | — |
| 2.062 | CH(CH₃) | CH₂ | CH₂ | H | H | H | CHF₂ | — |
| 2.063 | CH(CH₃) | CH₂ | CH₂ | H | H | H | CF₃ | — |
| 2.064 | CH(CH₃) | CH₂ | CH₂ | H | H | H | NO₂ | — |
| 2.065 | CH(CH₃) | CH₂ | CH₂ | H | H | H | OCHF₂ | — |
| 2.066 | CH(CH₃) | CH₂ | CH₂ | H | H | H | OCF₃ | — |
| 2.067 | CH(CH₃) | CH₂ | CH₂ | H | H | H | Ph | — |
| 2.068 | CH(CH₃) | CH₂ | CH₂ | H | H | H | NH₂ | — |
| 2.069 | CH(CH₃) | CH₂ | CH₂ | H | H | H | NHCOCH₃ | — |
| 2.070 | CH(CH₃) | CH₂ | CH₂ | H | F | H | H | — |
| 2.071 | CH(CH₃) | CH₂ | CH₂ | H | Cl | H | H | — |
| 2.072 | CH(CH₃) | CH₂ | CH₂ | H | Br | H | H | — |
| 2.073 | CH(CH₃) | CH₂ | CH₂ | H | I | H | H | — |
| 2.074 | CH(CH₃) | CH₂ | CH(CH₃) | F | H | H | H | — |
| 2.075 | CH(CH₃) | CH₂ | CH(CH₃) | Cl | H | H | H | — |
| 2.076 | CH(CH₃) | CH₂ | CH(CH₃) | Br | H | H | H | — |
| 2.077 | CH(CH₃) | CH₂ | CH(CH₃) | I | H | H | H | — |
| 2.078 | CH(CH₃) | CH₂ | CH(CH₃) | CH₃ | H | H | H | — |
| 2.079 | CH(CH₃) | CH₂ | CH(CH₃) | CH₂F | H | H | H | — |
| 2.080 | CH(CH₃) | CH₂ | CH(CH₃) | CHF₂ | H | H | H | — |
| 2.081 | CH(CH₃) | CH₂ | CH(CH₃) | CF₃ | H | H | H | — |
| 2.082 | CH(CH₃) | CH₂ | CH(CH₃) | NO₂ | H | H | H | — |
| 2.083 | CH(CH₃) | CH₂ | CH(CH₃) | OCHF₂ | H | H | H | — |
| 2.084 | CH(CH₃) | CH₂ | CH(CH₃) | OCF₃ | H | H | H | — |
| 2.085 | CH(CH₃) | CH₂ | CH(CH₃) | NHCOCH₃ | H | H | H | — |
| 2.086 | CH(CH₃) | CH₂ | CH(CH₃) | Ph | H | H | H | — |
| 2.087 | C(CH₃)₂ | CH₂ | CH₂ | F | H | H | H | Ret = 1.31 |
| 2.088 | C(CH₃)₂ | CH₂ | CH₂ | Cl | H | H | H | — |
| 2.089 | C(CH₃)₂ | CH₂ | CH₂ | Br | H | H | H | — |
| 2.090 | C(CH₃)₂ | CH₂ | CH₂ | I | H | H | H | — |
| 2.091 | C(CH₃)₂ | CH₂ | CH₂ | CH₃ | H | H | H | Ret = 1.34 |
| 2.092 | C(CH₃)₂ | CH₂ | CH₂ | CH₂F | H | H | H | — |
| 2.093 | C(CH₃)₂ | CH₂ | CH₂ | CHF₂ | H | H | H | — |
| 2.094 | C(CH₃)₂ | CH₂ | CH₂ | CF₃ | H | H | H | — |
| 2.095 | C(CH₃)₂ | CH₂ | CH₂ | NO₂ | H | H | H | — |
| 2.096 | C(CH₃)₂ | CH₂ | CH₂ | OCHF₂ | H | H | H | — |
| 2.097 | C(CH₃)₂ | CH₂ | CH₂ | OCF₃ | H | H | H | — |
| 2.098 | C(CH₃)₂ | CH₂ | CH₂ | Ph | H | H | H | — |
| 2.099 | C(CH₃)₂ | CH₂ | CH₂ | NH₂ | H | H | H | — |
| 2.100 | C(CH₃)₂ | CH₂ | CH₂ | NHCOCH₃ | H | H | H | — |
| 2.101 | C(CH₃)₂ | CH₂ | CH₂ | NHCOCF₃ | H | H | H | — |
| 2.102 | CH₂ | CH(CH₃) | CH₂ | F | H | H | H | 119-121 |
| 2.103 | CH₂ | CH(CH₃) | CH₂ | Cl | H | H | H | — |
| 2.104 | CH₂ | CH(CH₃) | CH₂ | Br | H | H | H | — |
| 2.105 | CH₂ | CH(CH₃) | CH₂ | I | H | H | H | — |
| 2.106 | CH₂ | CH(CH₃) | CH₂ | CH₃ | H | H | H | Ret = 1.25 |
| 2.107 | CH₂ | CH(CH₃) | CH₂ | CH₂F | H | H | H | — |
| 2.108 | CH₂ | CH(CH₃) | CH₂ | CHF₂ | H | H | H | — |
| 2.109 | CH₂ | CH(CH₃) | CH₂ | CF₃ | H | H | H | — |
| 2.110 | CH₂ | CH(CH₃) | CH₂ | NO₂ | H | H | H | — |
| 2.111 | CH₂ | CH(CH₃) | CH₂ | OCHF₂ | H | H | H | — |
| 2.112 | CH₂ | CH(CH₃) | CH₂ | OCF₃ | H | H | H | — |
| 2.113 | CH₂ | CH(CH₃) | CH₂ | Ph | H | H | H | — |
| 2.114 | CH₂ | CH(CH₃) | CH₂ | NH₂ | H | H | H | — |
| 2.115 | CH₂ | CH(CH₃) | CH₂ | NHCOCH₃ | H | H | H | — |
| 2.116 | CH₂ | CH(CH₃) | CH₂ | NHCOCF₃ | H | H | H | — |
| 2.117 | CHCO₂CH₃ | CH₂ | CH₂ | F | H | H | H | — |
| 2.118 | CHCO₂CH₃ | CH₂ | CH₂ | Cl | H | H | H | — |
| 2.119 | CHCO₂CH₃ | CH₂ | CH₂ | Br | H | H | H | — |
| 2.120 | CHCO₂CH₃ | CH₂ | CH₂ | I | H | H | H | — |
| 2.121 | CHCO₂CH₃ | CH₂ | CH₂ | CH₃ | H | H | H | — |
| 2.122 | CHCO₂CH₃ | CH₂ | CH₂ | CH₂F | H | H | H | — |
| 2.123 | CHCO₂CH₃ | CH₂ | CH₂ | CHF₂ | H | H | H | — |
| 2.124 | CHCO₂CH₃ | CH₂ | CH₂ | CF₃ | H | H | H | — |
| 2.125 | CHCO₂CH₃ | CH₂ | CH₂ | NO₂ | H | H | H | — |
| 2.126 | CHCO₂CH₃ | CH₂ | CH₂ | OCHF₂ | H | H | H | — |
| 2.127 | CHCO₂CH₃ | CH₂ | CH₂ | OCF₃ | H | H | H | — |
| 2.128 | CHCO₂CH₃ | CH₂ | CH₂ | Ph | H | H | H | — |
| 2.129 | CHCO₂CH₃ | CH₂ | CH₂ | NH₂ | H | H | H | — |
| 2.130 | CHCO₂CH₃ | CH₂ | CH₂ | NHCOCH₃ | H | H | H | — |
| 2.131 | CHCO₂CH₃ | CH₂ | CH₂ | NHCOCF₃ | H | H | H | — |
| 2.132 | CH(CH₃) | CH₂ | CH₂—CH₂ | CH₂F | H | H | H | — |
| 2.133 | CH(CH₃) | CH₂ | CH₂—CH₂ | CHF₂ | H | H | H | — |
| 2.134 | CH(CH₃) | CH₂ | CH₂—CH₂ | CF₃ | H | H | H | — |
| 2.135 | CH(CH₃) | CH₂ | CH₂—CH₂ | CCl₃ | H | H | H | — |
| 2.136 | CH(CH₃) | CH₂ | CH₂—CH₂ | NO₂ | H | H | H | — |
| 2.137 | CH(CH₃) | CH₂ | CH₂—CH₂ | CHO | H | H | H | — |
| 2.138 | CH(CH₃) | CH₂ | CH₂—CH₂ | CH₂OH | H | H | H | — |
| 2.139 | CH(CH₃) | CH₂ | CH₂—CH₂ | OH | H | H | H | — |
| 2.140 | CH(CH₃) | CH₂ | CH₂—CH₂ | NH₂ | H | H | H | — |
| 2.141 | CH(CH₃) | CH₂ | CH₂—CH₂ | NHCOCH₃ | H | H | H | — |
| 2.142 | CH(CH₃) | CH₂ | CH₂—CH₂ | OSO₂CF₃ | H | H | H | — |
| 2.143 | CH(CH₃) | CH₂ | CH₂—CH₂ | CH₃ | H | H | H | — |
| 2.144 | CH(CH₃) | CH₂ | CH₂—CH₂ | CO₂H | H | H | H | — |
| 2.145 | CH(CH₃) | CH₂ | CH₂—CH₂ | CO₂CH₃ | H | H | H | — |
| 2.146 | CH(CH₃) | CH₂ | CH₂—CH₂ | CN | H | H | H | — |
| 2.147 | CH(CH₃) | CH₂ | CH₂—CH₂ | CH═CH₂ | H | H | H | — |
| 2.148 | CH(CH₃) | CH₂ | CH₂—CH₂ | CCH | H | H | H | — |
| 2.149 | CH(CH₃) | CH₂ | CH₂—CH₂ | CONH₂ | H | H | H | — |
| 2.150 | CH(CH₃) | CH₂ | CH₂—CH₂ | N(CH₃)₂ | H | H | H | — |
| 2.151 | CH(CH₃) | CH₂ | CH₂—CH₂ | CH₂CH═CH₂ | H | H | H | — |
| 2.152 | CH(CH₃) | CH₂ | CH₂—CH₂ | COCH₃ | H | H | H | — |
| 2.153 | CH(CH₃) | CH₂ | CH₂—CH₂ | CF₂CH₃ | H | H | H | — |
| 2.154 | CH(CH₃) | CH₂ | CH₂—CH₂ | CHFCH₃ | H | H | H | — |
| 2.155 | CH(CH₃) | CH₂ | CH₂—CH₂ | OPh | H | H | H | — |
| 2.156 | CH(CH₃) | CH₂ | CH₂—CH₂ | Ph | H | H | H | — |
| 2.157 | CH(CH₃) | CH₂ | CH₂—CH₂ | SCH₃ | H | H | H | — |
| 2.158 | CH(CH₃) | CH₂ | CH₂—CH₂ | S(O)CH₃ | H | H | H | — |
| 2.159 | CH(CH₃) | CH₂ | CH₂—CH₂ | SO₂CH₃ | H | H | H | — |
| 2.160 | CH(CH₃) | CH₂ | CH₂—CH₂ | SCF₃ | H | H | H | — |
| 2.161 | CH(CH₃) | CH₂ | CH₂—CH₂ | SCHF₂ | H | H | H | — |
| 2.162 | CH(CH₃) | CH₂ | CH₂—CH₂ | S(O)CHF₂ | H | H | H | — |
| 2.163 | CH(CH₃) | CH₂ | CH₂—CH₂ | OCHF₂ | H | H | H | — |
| 2.164 | CH(CH₃) | CH₂ | CH₂—CH₂ | OCF₃ | H | H | H | — |
| 2.165 | CH(CH₃) | CH₂ | CH₂—CH₂ | OCH₃ | H | H | H | — |
| 2.166 | CH(CH₃) | CH₂ | CH₂—CH₂ | F | H | H | H | — |
| 2.167 | CH(CH₃) | CH₂ | CH₂—CH₂ | Cl | H | H | H | — |
| 2.168 | CH(CH₃) | CH₂ | CH₂—CH₂ | Br | H | H | H | — |
| 2.169 | CH(CH₃) | CH₂ | CH₂—CH₂ | I | H | H | H | — |
| 2.170 | CHC₂H₅ | CH₂ | CH₂ | F | H | H | H | Ret = 1.30 |
| 2.171 | CHC₂H₅ | CH₂ | CH₂ | F | H | F | H | Ret = 1.34 |
| 2.172 | CHC₂H₅ | CH₂ | CH₂ | CH₃ | H | H | H | Ret = 1.33 |
| 2.173 | CHnPr | CH₂ | CH₂ | F | H | H | H | |
| 2.174 | CHnPr | CH₂ | CH₂ | F | H | F | H | |
| 2.175 | CHnPr | CH₂ | CH₂ | CH₃ | H | H | H | |
| 2.176 | CHPh | CH₂ | CH₂ | F | H | H | H | |
| 2.177 | CHPh | CH₂ | CH₂ | F | H | F | H | |
| 2.178 | CHPh | CH₂ | CH₂ | CH₃ | H | H | H | |
| 2.179 | CHCH₂Ph | CH₂ | CH₂ | F | H | H | H | |
| 2.180 | CHCH₂Ph | CH₂ | CH₂ | F | H | F | H | |
| 2.181 | CHCH₂Ph | CH₂ | CH₂ | CH₃ | H | H | H | |
| 2.182 | CH-allyl | CH₂ | CH₂ | F | H | H | H | |
| 2.183 | CH-allyl | CH₂ | CH₂ | F | H | F | H | |
| 2.184 | CH-allyl | CH₂ | CH₂ | CH₃ | H | H | H | |
| 2.185 | CH- | CH₂ | CH₂ | F | H | H | H | |
| | propargyl | | | | | | | |
| 2.186 | CH- | CH₂ | CH₂ | F | H | F | H | |
| | propargyl | | | | | | | |
| 2.187 | CH- | CH₂ | CH₂ | CH₃ | H | H | H | |
| | propargyl | | | | | | | |
| 2.188 | CH₂ | C(CH₃)₂ | CH₂ | F | H | H | H | |
| 2.189 | CH₂ | C(CH₃)₂ | CH₂ | F | H | F | H | |
| 2.190 | CH₂ | C(CH₃)₂ | CH₂ | CH₃ | H | H | H | |
| 2.191 | CH₂ | CH(C₂H₅) | CH₂ | F | H | H | H | Ret = 1.33 |
| 2.192 | CH₂ | CH(C₂H₅) | CH₂ | F | H | F | H | Ret = 1.36 |
| 2.193 | CH₂ | CH(C₂H₅) | CH₂ | CH₃ | H | H | H | Ret = 1.35 |
| 2.194 | CH₂ | CH(nPr) | CH₂ | F | H | H | H | |
| 2.195 | CH₂ | CH(nPr) | CH₂ | F | H | F | H | |
| 2.196 | CH₂ | CH(nPr) | CH₂ | CH₃ | H | H | H | |
| 2.197 | CH₂ | CHPh | CH₂ | F | H | H | H | Ret = 1.50 |
| 2.198 | CH₂ | CHPh | CH₂ | F | H | F | H | Ret = 1.53 |
| 2.199 | CH₂ | CHPh | CH₂ | CH₃ | H | H | H | Ret = 1.52 |
| 2.200 | CH(CF₃) | CH₂ | CH₂ | F | H | H | H | Ret = 1.42 |
| 2.201 | CH(CH₃) | CHPh | CH₂ | F | H | F | H | Ret = 1.58 |
| 2.202 | CH-iBu ^{b)} | CH₂ | CH₂ | F | H | CH₃ | H | — |
| 2.203 | CH- | CH₂ | CH₂ | F | H | H | H | Ret = 1.44 |
| | (2-thienyl) | | | | | | | |
| 2.204 | CH(CH₂OH) | CH₂ | CH₂ | F | H | H | H | Ret = 1.12 |
| 2.205 | CH-iBu ^{b)} | CH(CH₃) | CH₂ | F | H | H | H | Ret = 1.50 |
| 2.206 | CH-iPr | CH₂ | CH₂ | F | H | H | H | Ret = 1.34 |
| 2.207 | C(CH₃)₂ | CH₂ | CH₂ | F | H | F | H | Ret = 1.36 |
| 2.208 | CH(CF₃) | CH₂ | CH₂ | F | H | F | H | Ret = 1.47 |
| 2.209 | CH₂ | CH(CH₃) | CH₂ | F | H | F | H | Ret = 1.27 |
| 2.210 | CH-iPr | CH₂ | CH₂ | F | H | F | H | Ret = 1.41 |
| 2.211 | CH₂ | CH(CH₂CH) | CH₂ | F | H | F | H | Ret = 1.16 |
| 2.212 | CH₂ | CH(CH₂CH) | CH₂ | F | H | H | H | Ret = 1.14 |
| 2.213 | CH- | CH₂ | CH₂ | F | H | F | H | Ret = 1.47 |
| | (2-thienyl) | | | | | | | |
| 2.214 | CH(CH₂OH) | CH₂ | CH₂ | F | H | F | H | Ret = 1.15 |
| 2.215 | CH-iBu ^{b)} | CH(CH₃) | CH₂ | F | H | F | H | Ret = 1.58 |
| 2.216 | CH(CF₃) | CH₂ | CH₂ | CH₃ | H | H | H | Ret = 1.49 |
| 2.217 | CH(CH₃) | CHPh | CH₂ | CH₃ | H | H | H | Ret = 1.59 |
| 2.218 | CH-iPr | CH₂ | CH₂ | CH₃ | H | H | H | Ret = 1.40 |
| 2.219 | CH₂ | CH—CH₂OPh | CH₂ | CH₃ | H | H | H | Ret = 1.57 |
| 2.220 | CH₂ | CH(CH₂OH) | CH₂ | CH₃ | H | H | H | Ret = 1.14 |
| 2.221 | CH- | CH₂ | CH₂ | CH₃ | H | H | H | Ret = 1.46 |
| | (2-thienyl) | | | | | | | |
| 2.222 | CH(CH₂OH) | CH₂ | CH₂ | CH₃ | H | H | H | Ret = 1.14 |
| 2.223 | CH-iBu ^{b)} | CH(CH₃) | CH₂ | CH₃ | H | H | H | solid |
| 2.224 | CH- | CH₂ | CH₂ | F | H | H | H | Ret = 1.21 |
| | (3-THF) ^{c)} | | | | | | | |
| 2.225 | CH- | CH₂ | CH₂ | F | H | F | H | Ret = 1.25 |
| | (3-THF) ^{c)} | | | | | | | |
| 2.226 | CH₂ | CH—CH₂OPh | CH₂ | F | H | H | H | Ret = 1.54 |

^{a)} The retention times (Ret) were obtained on an analytical HPLC column as follows:
A 3 × 20 mm 3 um dc18 Atlantis column was used. A gradient of water in acetonitrile was used. Each solvent contained 0.05% trifluoroacetic acid. There was a 3 minute run time for the analysis. From 0 minutes to 2.5 minutes a linear gradient starting from 5% acetonitrile to 100% acetonitrile was used, then a further 0.3 minutes of 100% acetonitrile, then over 0.1 minute the concentration of acetonitrile was reduced to 5%, ready for the next analysis. The compounds were detected with Waters detectors; ZQ2000, 2996PDA, 2420 ELSD.
^{b)} iBu = CH₂—CH(CH₃)₂.
^{c)} 3-THF = 3-tetrahydrofuranyl.

TABLE 3

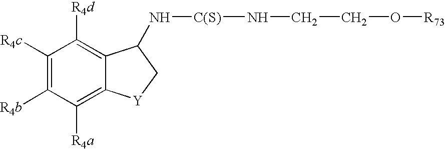

| Cpd. No | R₇₃ | Y | R₄a | R₄b | R₄c | R₄d | Mp [° C.]/Ret ^{a)} |
|---|---|---|---|---|---|---|---|
| 3.001 | COC(CH₃)₃ | CH₂ | CH₂F | H | H | H | — |
| 3.002 | COC(CH₃)₃ | CH₂ | CHF₂ | H | H | H | — |
| 3.003 | COC(CH₃)₃ | CH₂ | CH₃ | H | H | H | — |
| 3.004 | COC(CH₃)₃ | CH₂ | F | H | H | H | Ret = 1.7 |
| 3.005 | COC(CH₃)₃ | CH₂ | Cl | H | H | H | — |
| 3.006 | COC(CH₃)₃ | CH₂ | CH₂F | H | F | H | — |
| 3.007 | COC(CH₃)₃ | CH₂ | CHF₂ | H | F | H | — |
| 3.008 | COC(CH₃)₃ | CH₂ | CH₃ | H | F | H | — |
| 3.009 | COC(CH₃)₃ | CH₂ | F | H | F | H | — |
| 3.010 | COC(CH₃)₃ | CH₂ | H | H | H | H | Resin |
| 3.011 | COC(CH₃)₃ | CH₂—CH₂ | CH₂F | H | H | H | — |
| 3.012 | COC(CH₃)₃ | CH₂—CH₂ | CHF₂ | H | H | H | — |
| 3.013 | COC(CH₃)₃ | CH₂—CH₂ | CH₃ | H | H | H | — |
| 3.014 | COC(CH₃)₃ | CH₂—CH₂ | F | H | H | H | — |
| 3.015 | COC(CH₃)₃ | CH₂—CH₂ | Cl | H | H | H | — |
| 3.016 | COC(CH₃)₃ | CH₂—CH₂ | F | H | F | H | — |
| 3.017 | COC(CH₃)₃ | CH₂—CH₂ | CH₃ | H | F | H | — |
| 3.018 | COC(CH₃)₃ | CH₂ | H | F | H | H | — |
| 3.019 | COC(CH₃)₃ | CH₂ | H | Cl | H | H | — |
| 3.020 | COC(CH₃)₃ | CH₂ | H | CH₃ | H | H | — |
| 3.021 | COC(CH₃)₃ | CH₂ | H | H | H | F | — |
| 3.022 | COC(CH₃)₃ | CH₂ | H | H | H | Cl | — |
| 3.023 | COC(CH₃)₃ | CH₂ | H | H | H | CH₂F | — |
| 3.024 | COC(CH₃)₃ | CH₂ | H | H | H | CHF₂ | — |
| 3.025 | COC(CH₃)₃ | CH₂ | H | H | H | CH₃ | — |
| 3.026 | COC(CH₃)₃ | CH₂ | H | H | F | H | — |
| 3.027 | COC(CH₃)₃ | CH₂ | H | H | Cl | H | — |
| 3.028 | COC(CH₃)₃ | CH₂ | H | H | CHF₂ | H | — |
| 3.029 | COC(CH₃)₃ | CH₂ | H | H | CH₃ | H | — |
| 3.030 | COC₂H₅ | CH₂ | CH₃ | H | H | H | — |
| 3.031 | COC₂H₅ | CH₂ | F | H | H | H | solid |
| 3.032 | COC₂H₅ | CH₂ | Cl | H | H | H | — |
| 3.033 | COC₂H₅ | CH₂ | F | H | F | H | — |
| 3.034 | COPh | CH₂ | CH₃ | H | H | H | — |
| 3.035 | COPh | CH₂ | F | H | H | H | Ret = 1.7 |
| 3.036 | COPh | CH₂ | F | H | F | H | — |
| 3.037 | COCH₃ | CH₂ | CH₃ | H | H | H | — |
| 3.038 | COCH₃ | CH₂ | F | H | H | H | 137-138 |
| 3.039 | COCH₃ | CH₂ | F | H | F | H | — |
| 3.040 | C(O)cyclopropyl | CH₂ | F | H | H | H | Resin |
| 3.041 | C(O)cyclopropyl | CH₂ | F | H | F | H | |
| 3.042 | C(O)cyclopropyl | CH₂ | CH₃ | H | H | H | |
| 3.043 | C(O)OCH₃ | CH₂ | F | H | H | H | Ret = 1.3 |
| 3.044 | C(O)OCH₃ | CH₂ | F | H | F | H | |
| 3.045 | C(O)OCH₃ | CH₂ | CH₃ | H | H | H | |
| 3.046 | C(O)CO₂H₅ | CH₂ | F | H | H | H | solid |
| 3.047 | C(O)OC₂H₅ | CH₂ | F | H | F | H | |
| 3.048 | C(O)CO₂H₅ | CH₂ | CH₃ | H | H | H | |
| 3.049 | C(O)OPh | CH₂ | F | H | H | H | |
| 3.050 | C(O)OPh | CH₂ | F | H | F | H | |
| 3.051 | C(O)OPh | CH₂ | CH₃ | H | H | H | |
| 3.052 | C(O)NHCH₃ | CH₂ | F | H | H | H | |
| 3.053 | C(O)NHCH₃ | CH₂ | F | H | F | H | |
| 3.054 | C(O)NHCH₃ | CH₂ | CH₃ | H | H | H | |
| 3.055 | C(O)NHC₂H₅ | CH₂ | F | H | H | H | |
| 3.056 | C(O)NHC₂H₅ | CH₂ | F | H | F | H | |
| 3.057 | C(O)NHC₂H₅ | CH₂ | CH₃ | H | H | H | |
| 3.058 | C(O)N(CH₃)₂ | CH₂ | F | H | H | H | |
| 3.059 | C(O)N(CH₃)₂ | CH₂ | F | H | F | H | |
| 3.060 | C(O)N(CH₃)₂ | CH₂ | CH₃ | H | H | H | |
| 3.061 | C(O)NHPh | CH₂ | F | H | H | H | |
| 3.062 | C(O)NHPh | CH₂ | F | H | F | H | |
| 3.063 | C(O)NHPh | CH₂ | CH₃ | H | H | H | |
| 3.064 | COCH₃ | CH₂ | CH₃ | H | F | H | Resin |
| 3.065 | CO-nPr | CH₂ | F | H | H | H | Resin |
| 3.066 | CO-iPr | CH₂ | F | H | H | H | Resin |
| 3.067 | COCH₂OCH₃ | CH₂ | F | H | H | H | Resin |
| 3.068 | COCH₃ | CH₂ | F | H | CH₃ | H | Resin |
| 3.069 | COC₂H₅ | CH₂ | F | H | CH₃ | H | Solid |
| 3.070 | CO-nPr | CH₂ | F | H | CH₃ | H | Resin |
| 3.071 | CO-iPr | CH₂ | F | H | CH₃ | H | Resin |
| 3.072 | COC₂H₅ | CH₂ | CH₃ | H | F | H | Resin |
| 3.073 | CO-nPr | CH₂ | CH₃ | H | F | H | Resin |
| 3.074 | CO-iPr | CH₂ | CH₃ | H | F | H | Resin |
| 3.075 | CH₃ | CH₂ | H | H | H | H | Oil |
| 3.076 | CH₂OC₂H₅ | CH₂ | F | H | H | H | Resin |
| 3.077 | CH₃ | CH₂ | F | H | H | H | Oil |
| 3.078 | CH₃ | CH₂ | CH₃ | H | H | H | Oil |
| 3.079 | 2-THP ^{b)} | CH₂ | H | H | H | H | Resin |
| 3.080 | COCHCH₃(C₂H₅) | CH₂ | F | H | H | H | Ret = 1.6 |
| 3.081 | COCH₂CO₂CH₃ | CH₂ | F | H | H | H | Ret = 1.3 |
| 3.082 | COCH₂OCOCH₃ | CH₂ | F | H | H | H | Ret = 1.3 |
| 3.083 | COCH₂CH₂SCH₃ | CH₂ | F | H | H | H | Ret = 1.5 |
| 3.084 | COCH₂CH₂CH₂Cl | CH₂ | F | H | H | H | Ret = 1.6 |
| 3.085 | CO-(2-furanyl) | CH₂ | F | H | H | H | Ret = 1.4 |
| 3.086 | CO-cC₆H₁₁ | CH₂ | F | H | H | H | Ret = 1.8 |
| 3.087 | CO(CH₂)₅CH₃ | CH₂ | F | H | H | H | Ret = 1.9 |
| 3.088 | COCH₂C(CH₃)₃ | CH₂ | F | H | H | H | Ret = 1.8 |
| 3.089 | COCH₂-Ph | CH₂ | F | H | H | H | Ret = 1.7 |
| 3.090 | COCH₂CH₂CO₂H | CH₂ | F | H | H | H | Ret = 1.1 |
| 3.091 | CO(CH₂)₄—Cl | CH₂ | F | H | H | H | Ret = 1.6 |
| 3.092 | COC(CH₃)₂CH₂Cl | CH₂ | F | H | H | H | Ret = 1.7 |
| 3.093 | CO-(2-thienyl) | CH₂ | F | H | H | H | Ret = 1.6 |
| 3.094 | CO(CH₂)₆CH₃ | CH₂ | F | H | H | H | Ret = 2.0 |
| 3.095 | CO(CH₂)₂CO₂C₂H₅ | CH₂ | F | H | H | H | Ret = 1.5 |
| 3.096 | CO(CH₂)₃CO₂CH₃ | CH₂ | F | H | H | H | Ret = 1.4 |
| 3.097 | COCH₂CH₂-Ph | CH₂ | F | H | H | H | Ret = 1.7 |
| 3.098 | CO(CH₂)₃CO₂H | CH₂ | F | H | H | H | Ret = 1.2 |
| 3.099 | COCH₂OPh | CH₂ | F | H | H | H | Ret = 1.7 |
| 3.100 | CO(CH₂)₇CH₃ | CH₂ | F | H | H | H | Ret = 2.2 |
| 3.101 | CO-(3-pyridyl) | CH₂ | F | H | H | H | Ret = 1.3 |
| 3.102 | CO(CH₂)₄CO₂H | CH₂ | F | H | H | H | Ret = 1.3 |
| 3.103 | CO(CH₂)₈CH₃ | CH₂ | F | H | H | H | Ret = 2.3 |
| 3.104 | CO(CH₂)₇CH₂CH═CH₂ | CH₂ | F | H | H | H | Ret = 2.3 |
| 3.105 | CO(CH₂)₅CO₂C₂H₅ | CH₂ | F | H | H | H | Ret = 1.7 |
| 3.106 | CO(CH₂)₆CO₂CH₃ | CH₂ | F | H | H | H | Ret = 1.7 |
| 3.107 | COC(CH₃)₂CO₂CH₃ | CH₂ | F | H | H | H | Ret = 1.5 |
| 3.108 | CO-(1-adamantyl) | CH₂ | F | H | H | H | Ret = 2.09 |
| 3.109 | COO(CH₂)₄Cl | CH₂ | F | H | H | H | Ret = 1.7 |
| 3.110 | CO(CH₂)₁₀CH₃ | CH₂ | F | H | H | H | Ret = 2.5 |
| 3.111 | COC(CH₃)═CH₂ | CH₂ | F | H | H | H | Ret = 1.8 |
| 3.112 | CO(CH₂)₃CO₂C₂H₅ | CH₂ | F | H | H | H | Ret = 1.5 |
| 3.113 | COCH₂CO₂C₂H₅ | CH₂ | F | H | H | H | Ret = 1.4 |
| 3.114 | COCH═C(CH₃)₂ | CH₂ | F | H | H | H | Ret = 1.6 |
| 3.115 | COCH₂CH(CH₃)CH₂C(CH₃)₃ | CH₂ | F | H | H | H | Ret = 2.1 |
| 3.116 | COC(CH₃)₂OAc | CH₂ | F | H | H | H | Resin |
| 3.117 | COCH₂-2-thienyl | CH₂ | F | H | H | H | Resin |
| 3.118 | OSi(Ph)₂C(CH₃)₃ | CH₂ | H | H | H | H | Resin |

^{a)} Ret = Retention time in minutes. HPLC-data were performed with a Waters 2795 HPLC using an Atlantis dC18 3 μm, 3 × 20 mm column. Eluent-system: 80% water, 20% acetonitril, 0.1% formic acid, linear gradient from 20% acetonitril to 100% acetonitril within 2.5 mm. The flow rate was 1.7 ml/min. Compounds were detected by UV using a diode array measuring wavelengths from 200 nm to 400 nm.
^{b)} 2-THP = 2-tetrahydo-pyranyl.

TABLE 4

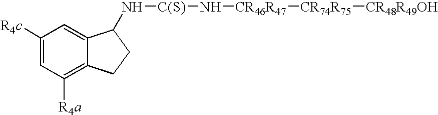

| Cpd. | | | | | | Mp [° C.]/ |
|---|---|---|---|---|---|---|
| No | R4a | R4c | CR₄₆R₄₇ | CR₇₄R₇₅ | CR₄₈R₄₉ | Ret ^{a)} |
| 4.001 | F | H | CH₂ | C(CH₃)₂ | CH₂ | Ret = 1.39 |
| 4.002 | F | F | CH₂ | CH₂ | CH₂ | Ret = 1.24 |
| 4.003 | F | F | CH₂ | C(CH₃)₂ | CH₂ | Ret = 1.44 |
| 4.004 | CH₃ | H | CH₂ | CH₂ | CH₂ | Ret = 1.22 |
| 4.005 | CH₃ | H | CH₂ | C(CH₃)₂ | CH₂ | Ret = 1.43 |
| 4.006 | F | H | CH₂ | CH₂ | CH₂ | 125-127 |

^{a)} Ret = HPLC-retention time in minutes, for experimental details see table 2.

TABLE 5

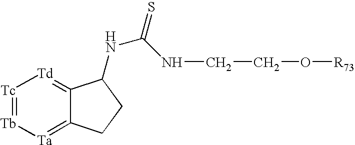

| Cpd. No. | Ta | Tb | Tc | Td | R₇₃ | Phys. Data |
|---|---|---|---|---|---|---|
| 5.001 | CCH₃ | CH | CCl | N | H | 159-160° C. |
| 5.002 | CCF₃ | CH | CCl | N | H | resin *) |
| 5.003 | CCF₃ | CH | CMe | N | H | resin *) |
| 5.004 | CCH₃ | CH | N | CH | H | 175-185 |

*) ¹H-NMR (CDCl₃) of selected compounds:

| | |
|---|---|
| 5.002 | 7.75, b, 1H; 7.62, b, 1H; 7.40, s, 1H; 6.00, m, 1H; 3.8-4.1, |
| | m, 3H; 3.77, m; 1H; 3.18, m, 1H; 2.98, m, 2H; 2.02, m, 1H. |
| 5.003 | 8.5, b, 1H; 7.24, s, 1H; 6.7, b, 1H; 5.33, m, 1H; 3.90, m, 1H; |
| | 3.82, m; 1H; 3.18, m, 1H; 3.02, m, 1H; 2.74, m, 1H; 2.60, s, |
| | 3H; 2.04, m, 1H. |

TABLE 6

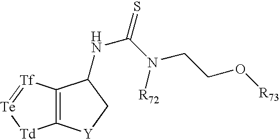

| Cpd. No | Td | Te | Tf | Y | R₇₂ | R₇₃ | Mp [° C.] |
|---|---|---|---|---|---|---|---|
| 6.001 | S | CH | CH | CH₂ | H | H | 110-111 |
| 6.002 | S | CCH₃ | CH | CH₂ | H | H | 122-124 |
| 6.003 | S | CH | CH | CH₂CH₂ | H | H | — |

TABLE 7

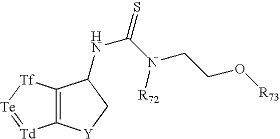

| Cpd. No | Td | Te | Tf | Y | R₇₂ | R₇₃ | Mp [° C.] |
|---|---|---|---|---|---|---|---|
| 7.001 | CH | CH | NCH₃ | CH₂ | H | H | oil |
| 7.002 | CH | CH | S | CH₂ | H | H | — |

TABLE 8

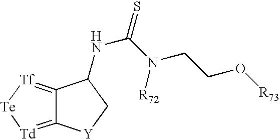

| Cpd. No | Td | Te | Tf | Y | R₇₂ | R₇₃ | Mp [° C.] |
|---|---|---|---|---|---|---|---|
| 8.001 | CCl | S | CCl | CH₂ | H | H | 143-145 |
| 8.002 | CH | S | CH | CH₂ | H | H | 122-124 |
| 8.003 | CCl | S | CH | CH₂ | H | H | 123-125 |

TABLE 9

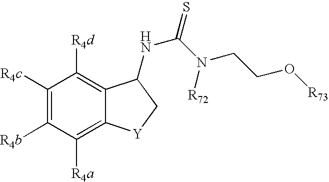

| Cpd. No | R₄ₐ | R_{4b} | R_{4c} | R_{4d} | Y | R₇₂ | R₇₃ | Mp [° C.] |
|---|---|---|---|---|---|---|---|---|
| 9.001 | F | H | F | H | CH₂ | NH₂ | H | 151.5-151 |

TABLE 10

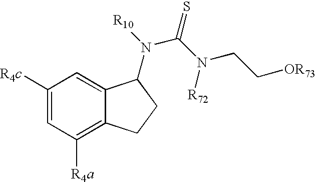

| Cpd. No | R₄ₐ | R_{4c} | R₁₀ | R₇₂ | R₇₃ | Mp [° C.]/Ret ^{a)} |
|---|---|---|---|---|---|---|
| 10.001 | H | H | H | CH₃ | H | oil |
| 10.002 | F | H | H | CH₃ | H | oil |
| 10.003 | CH₃ | H | H | CH₃ | H | oil |
| 10.004 | H | H | H | CH₃ | CH₃ | oil |
| 10.005 | F | H | H | CH₃ | CH₃ | oil |
| 10.006 | CH₃ | H | H | CH₃ | CH₃ | oil |
| 10.007 | H | H | H | CO—C(CH₃)₃ | CO—C(CH₃)₃ | resin |
| 10.008 | CH₃ | F | H | CO-(i-Pr) | CO-(i-Pr) | resin |
| 10.009 | F | H | H | CO-Ph | CO-Ph | 139-140 |
| 10.010 | F | H | CO-Ph | H | CO-Ph | resin |
| 10.011 | H | H | H | CH₂-(3-pyridyl) | H | resin |
| 10.012 | F | H | H | CH₂-(3-pyridyl) | H | 63-64 |
| 10.013 | CH₃ | H | H | CH₂-(3-pyridyl) | H | 127-128 |
| 10.014 | H | H | H | CH₂-(4-pyridyl) | H | 110-112 |
| 10.015 | F | H | H | CH₂-(4-pyridyl) | H | 133-135 |
| 10.016 | CH₃ | H | H | CH₂-(4-pyridyl) | H | 117-118 |
| 10.017 | F | H | H | CO-cyclopropyl | CO-cyclopropyl | Ret = 1.8 |
| 10.018 | F | H | H | CO-(n-Pr) | CO-(n-Pr) | Ret = 1.5 |
| 10.019 | F | H | H | CO—CH(CH₃)C₂H₅ | CO—CH(CH₃)C₂H₅ | Ret = 2.2 |
| 10.020 | F | H | CO—CH₂CH(CH₃)₂ | H | CO—CH₂CH(CH₃)₂ | Ret = 2.0 |
| 10.021 | F | H | H | CO—CH₂CH(CH₃)₂ | CO—CH₂CH(CH₃)₂ | Ret = 2.2 |
| 10.022 | F | H | CO—CH₂C(CH₃)₃ | H | CO—CH₂C(CH₃)₃ | Ret = 2.3 |
| 10.023 | F | H | H | CO—CH₂C(CH₃)₃ | CO—CH₂C(CH₃)₃ | Ret = 2.4 |
| 10.024 | F | H | H | CO-(c-C₆H₁₁) | CO-(c-C₆H₁₁) | Ret = 2.4 |
| 10.025 | F | H | H | 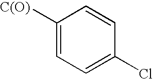 | 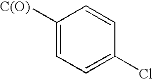 | Ret = 2.4 |
| 10.026 | F | H | H | 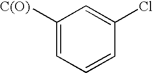 | 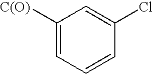 | Ret = 2.3 |
| 10.027 | F | H | H | 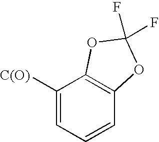 | 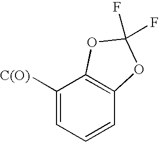 | Ret = 2.3 |
| 10.028 | F | H | H | 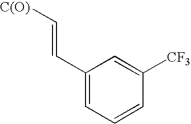 | 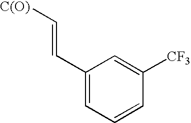 | Ret = 2.5 |
| 10.029 | F | H | 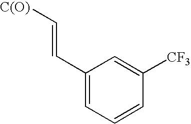 | H | 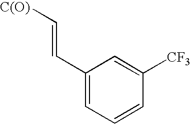 | Ret = 2.5 |
| 10.030 | F | H | H | 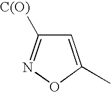 | 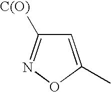 | Ret = 1.7 |
| 10.031 | F | H | H | 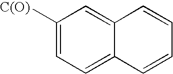 | 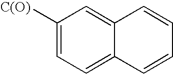 | Ret = 1.9 |
| 10.032 | F | H | H | COCH₃ | COCH₃ | oil |
| 10.033 | F | H | COCH₃ | H | COCH₃ | 110-111 |
| 10.034 | F | H | CO-cyclopropyl | H | CO-cyclopropyl | Resin |

^{a)} Retention time, for further experimental details see table 3.

TABLE 11
Characterising data of selected compounds.

| Cpd. No | Stucture | Mp [° C.] | Ret [min] a) | Ret [min] b) |
|---|---|---|---|---|
| 11.001 | 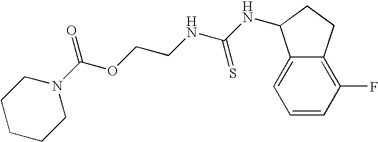 | | 1.5 | |
| 11.002 | 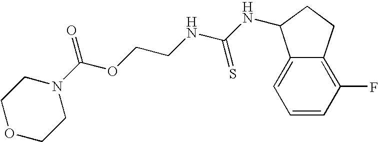 | | 1.2 | |
| 11.003 | 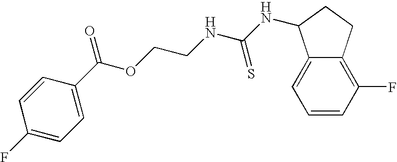 | | 1.7 | |
| 11.004 | 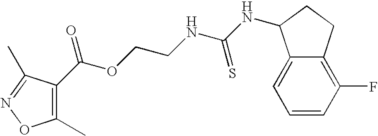 | | 1.5 | |
| 11.005 | 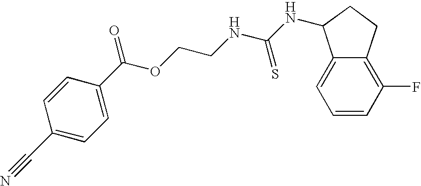 | | 1.6 | |
| 11.006 | 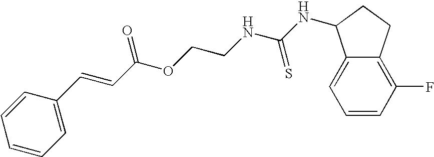 | | 1.8 | |
| 11.007 | 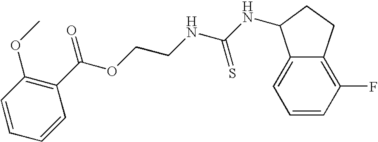 | | 1.6 | |
| 11.008 | 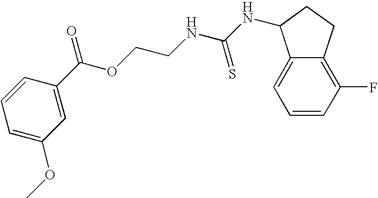 | | 1.7 | |
| 11.009 | 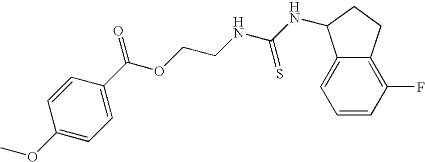 | | 1.7 | |
| 11.010 | 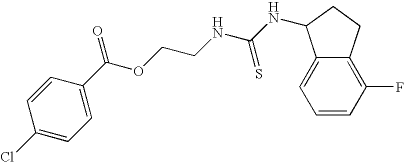 | | 1.9 | |
| 11.011 | 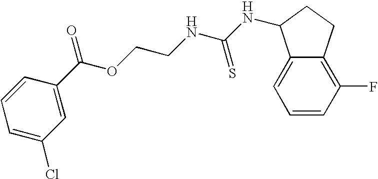 | | 1.8 | |
| 11.012 | 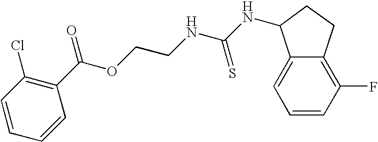 | | 1.8 | |
| 11.013 | 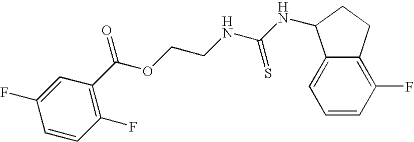 | | 1.7 | |
| 11.014 | 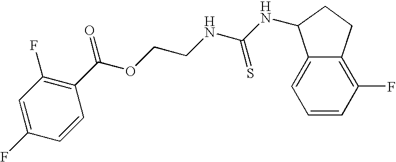 | | 1.7 | |
| 11.015 | 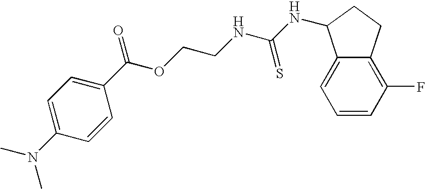 | | 1.8 | |
| 11.016 | 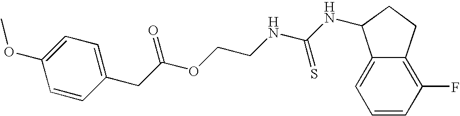 | | 1.7 | |
| 11.017 | 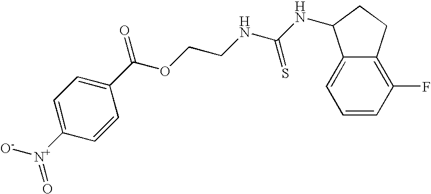 | | 1.7 | |
| 11.018 | 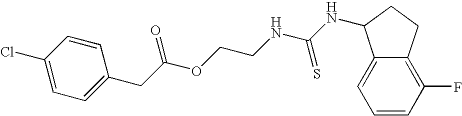 | | 1.8 | |
| 11.019 | 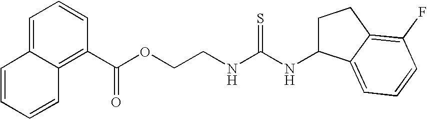 | | 1.9 | |
| 11.020 | 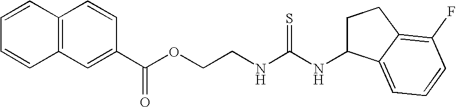 | | 1.9 | |
| 11.021 | 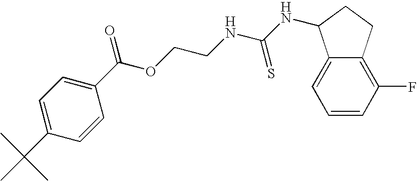 | | 2.1 | |
| 11.022 | 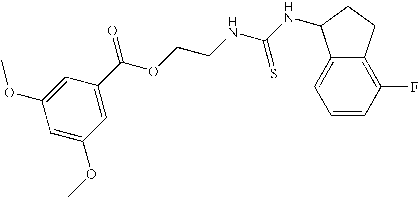 | | 1.7 | |
| 11.023 | 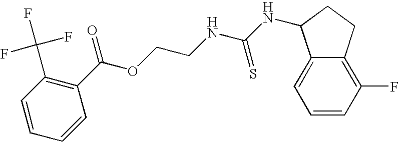 | | 1.8 | |
| 11.024 | 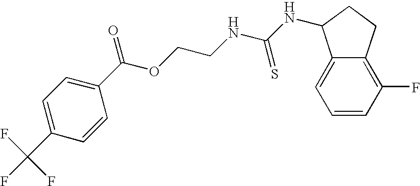 | | 1.9 | |
| 11.025 | 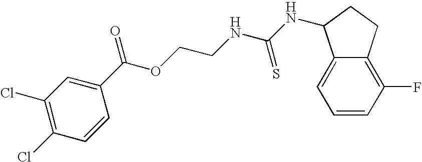 | | 2.0 | |
| 11.026 | 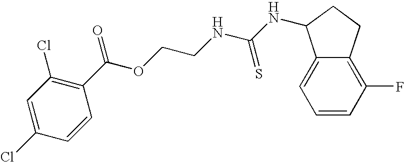 | | 1.9 | |
| 11.027 | 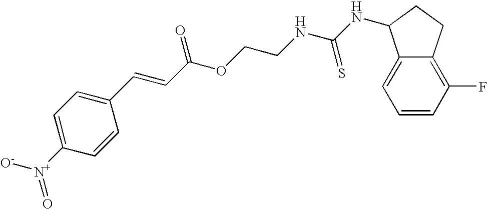 | | 1.8 | |
| 11.028 | 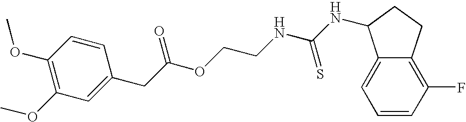 | | 1.6 | |
| 11.029 | 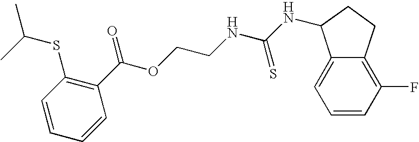 | | 1.9 | |
| 11.030 | 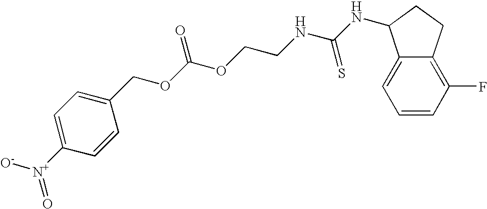 | | 1.7 | |
| 11.031 | 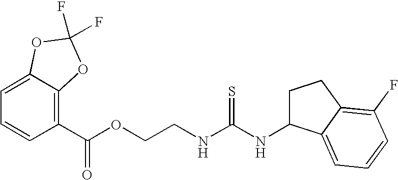 | | 1.9 | |
| 11.032 | 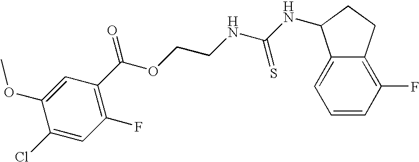 | | 1.8 | |
| 11.033 | 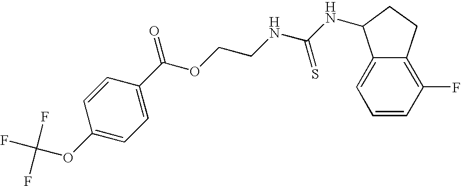 | | 1.9 | |
| 11.034 | 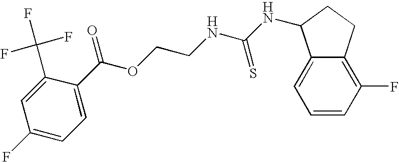 | | 1.9 | |
| 11.035 | 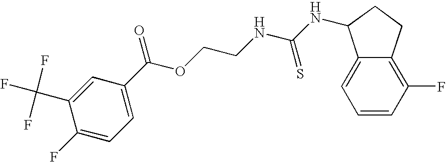 | | 1.9 | |
| 11.036 | 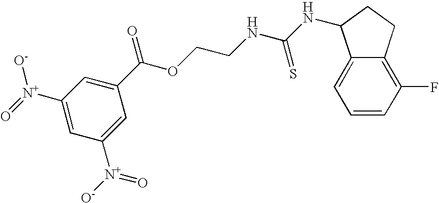 | | 1.7 | |
| 11.037 | 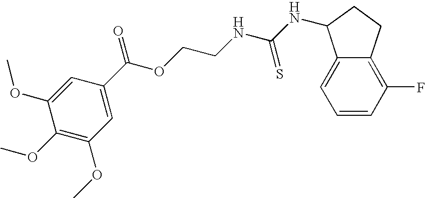 | | 1.6 | |
| 11.038 | 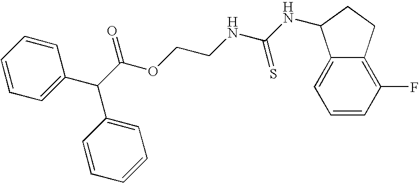 | | 2.0 | |
| 11.039 | 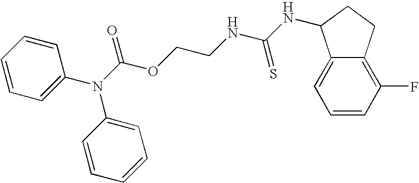 | | 1.9 | |
| 11.040 | 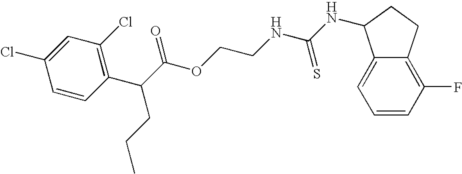 | | 2.3 | |
| 11.041 | 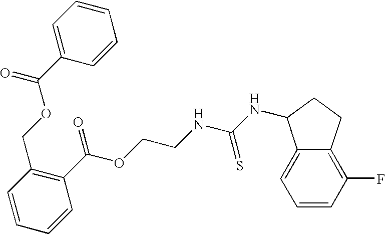 | | 2.0 | |
| 11.042 | 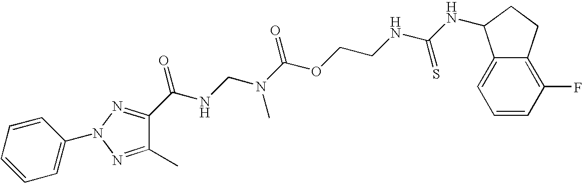 | | 1.8 | |
| 11.043 | 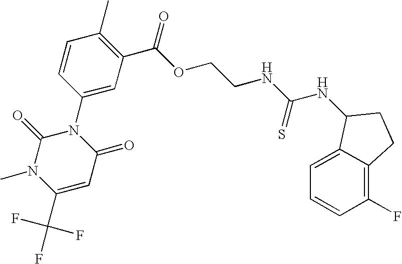 | | 1.8 | |
| 11.044 | 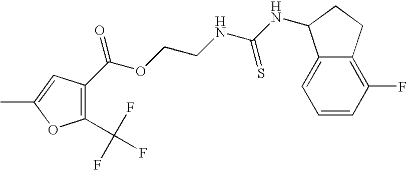 | | 1.9 | |
| 11.045 | 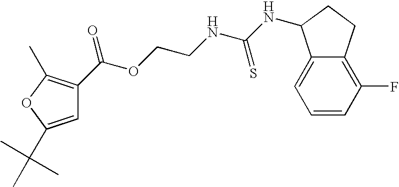 | | 2.1 | |
| 11.046 | 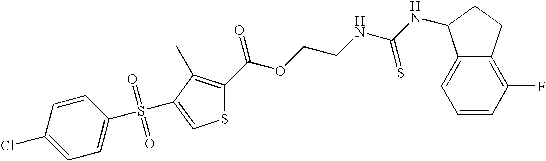 | | 2.0 | |
| 11.047 | 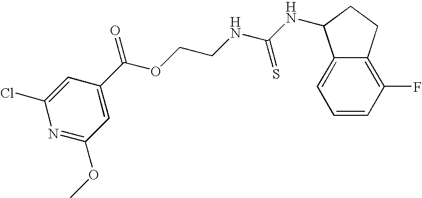 | | 1.8 | |
| 11.048 | 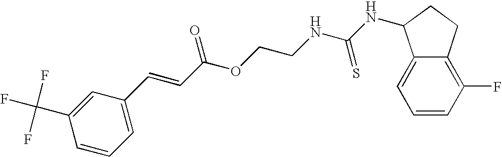 | | 1.9 | |
| 11.049 | 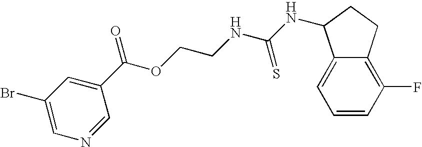 | | 1.6 | |
| 11.050 | 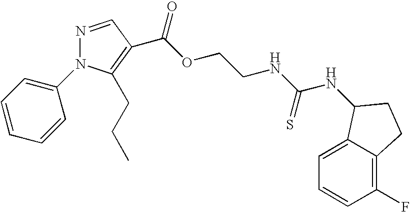 | | 1.9 | |
| 11.051 | 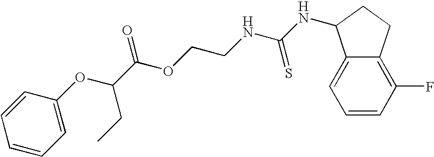 | | 1.8 | |
| 11.052 | 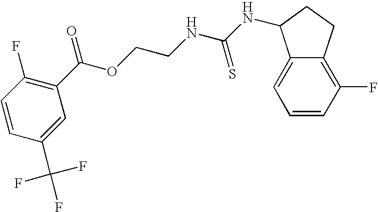 | | 1.8 | |
| 11.053 | 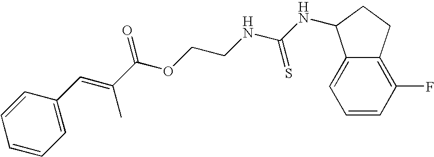 | | 1.8 | |
| 11.054 | 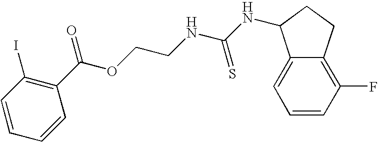 | | 1.8 | |
| 11.055 | 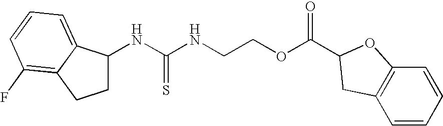 | | 1.7 | |
| 11.056 | 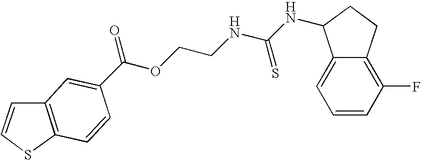 | | 1.8 | |
| 11.057 | 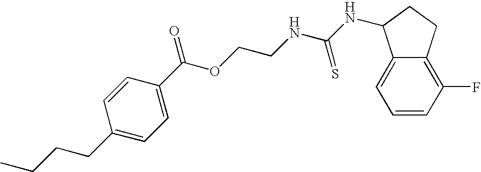 | | 2.1 | |
| 11.058 | 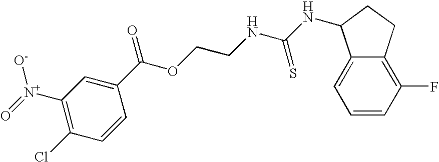 | | 1.8 | |
| 11.059 | 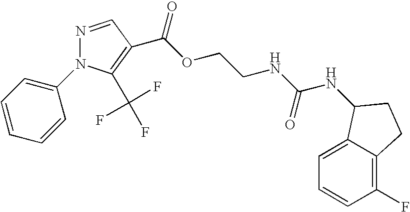 | | 1.9 | |
| 11.060 | 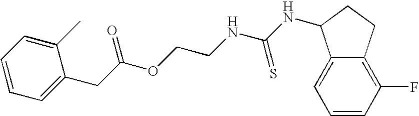 | | 1.7 | |
| 11.061 | 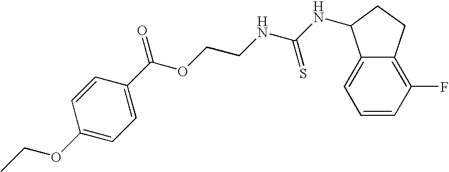 | | 1.8 | |
| 11.062 | 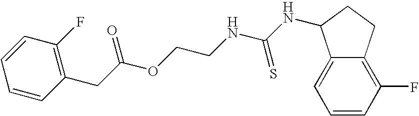 | | 1.7 | |
| 11.063 | 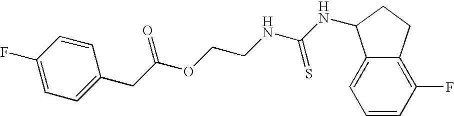 | | 1.7 | |
| 11.064 | 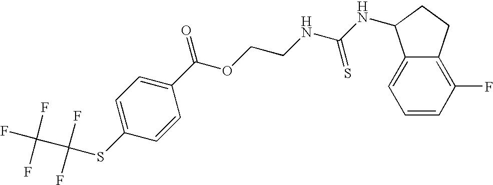 | | 2.1 | |
| 11.065 | 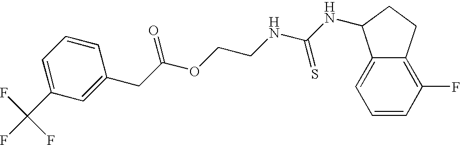 | | 1.9 | |
| 11.066 | 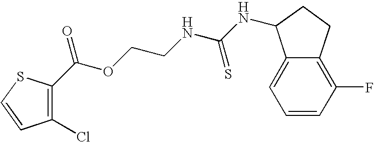 | | 1.7 | |
| 11.067 | 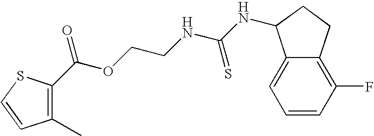 | | 1.7 | |
| 11.068 | 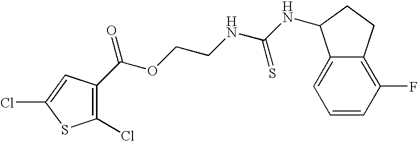 | | 2.0 | |
| 11.069 | 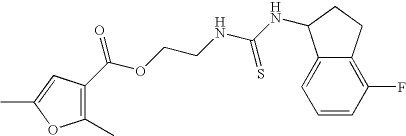 | | 1.8 | |
| 11.070 | 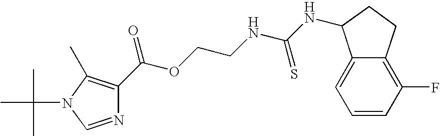 | | 1.7 | |
| 11.071 | 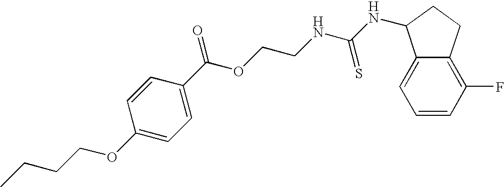 | | 2.1 | |
| 11.072 | 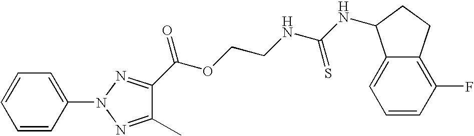 | | 1.9 | |
| 11.073 | 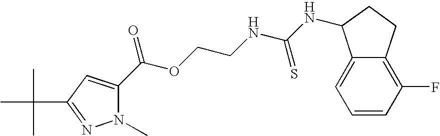 | | 1.8 | |
| 11.074 | 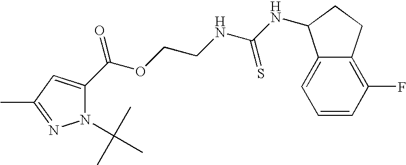 | | 1.7 | |
| 11.075 | 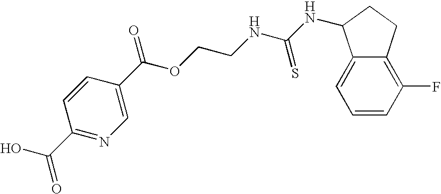 | | 1.2 | |
| 11.076 | 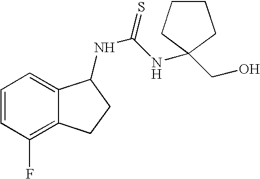 | | | 1.41 |
| 11.077 | 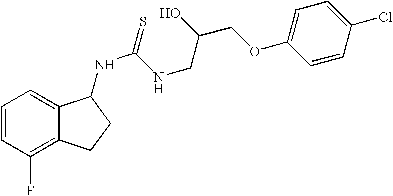 | | | 1.63 |
| 11.078 | 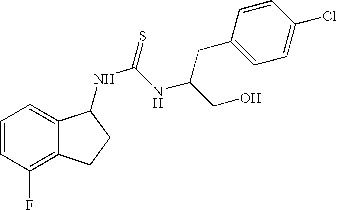 | | | 1.60 |
| 11.079 | 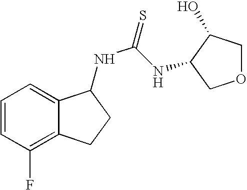 | | | |
| 11.080 | 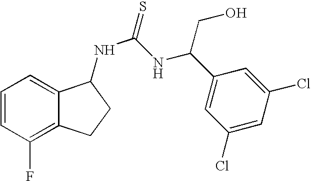 | | | 1.67 |
| 11.081 | 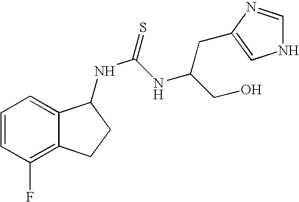 | | | 1.02 |
| 11.082 | 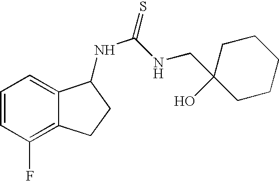 | | | 1.63 |

a) Ret = HPLC-retention time in minute, for details see table 3.
b) For details see table 2.

The compounds of the invention may be made by a variety of methods.

Compounds of formula (I) can be prepared by treatment of an amine of formula (IIa) with thiophosgene to afford an intermediate compound of formula (IIIa), which is then further reacted with an amine of formula (IVa) as outlined in the reaction scheme below (see Houben-Weyl 1983, E4, 484 and Chem. Ber. 1970, 103, 133).

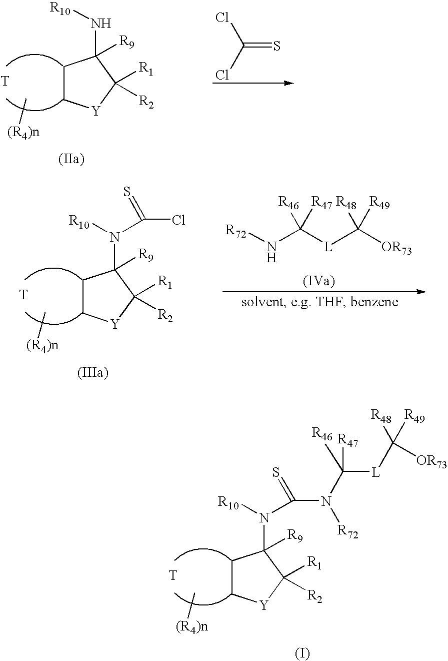

Furthermore, reaction of the isothiocyanates of formula (III) shown below with amine derivatives of formula (IVa) lead to thioureas of formula (Ia) wherein R₁₀ is hydrogen (see Tetrahedron Letters 2003, 44, 795 and Bull. Kor. Chem. Soc. 2000, 21, 919).

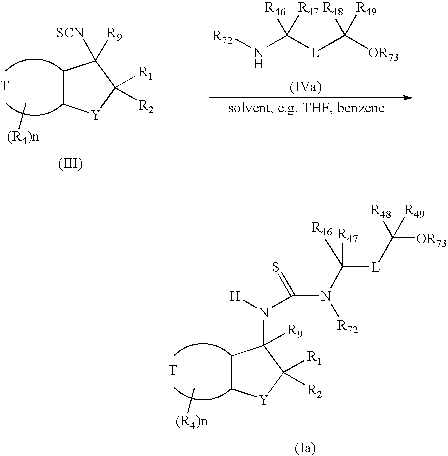

The isothiocyanates of formula (III) can be prepared in various ways. For example many methods for the preparation of isothiocyanates are reviewed by Sharma (Sulfur Reports 1988, 8, 327). Isothiocyanates (III) can be prepared from the amines (IIa) (J. Am. Chem. Soc. 2004, 126, 7450, Bioorg. & Med. Chem., 2003, 11, 4189 and Rec. Trav. Chim 1926, 45, 421), alcohols (IIb) (Chemistry Letters, 2006, 35(11), 1262 and Synthesis 2004 (1), 92) and azides (IIc) (Carbohyd. Res. 2002, 337, 2329) as shown in the reaction scheme below. They can be prepared by addition of thiocyanate to indene (IIf) (Tet. Lett. 1992, 33(25), 3599-3602).

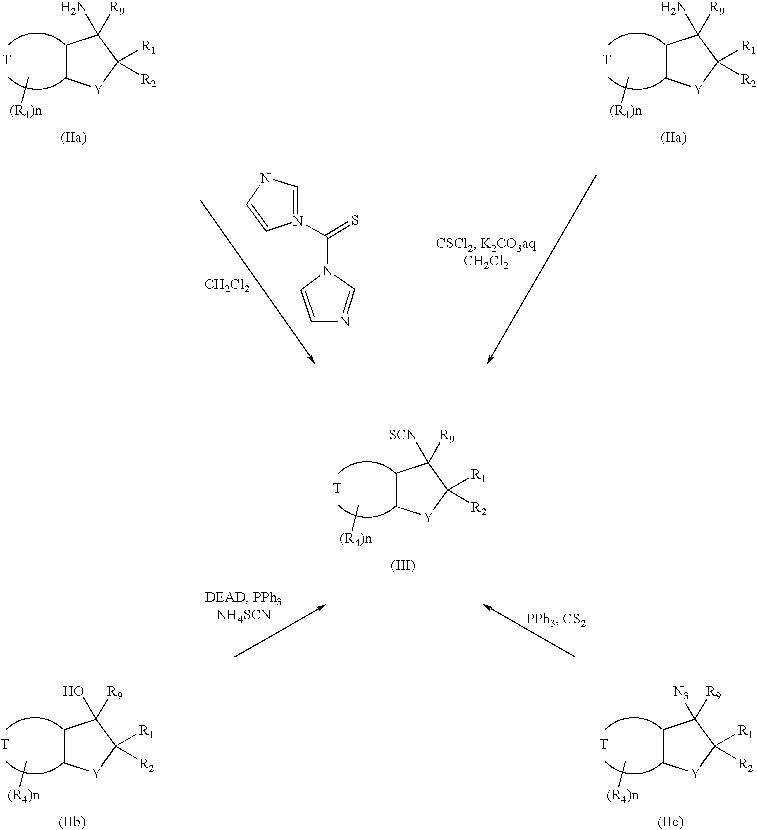

Further methods involve treating of an activated precursor (IId) (X=leaving group e.g. halogen, tosylate or mesylate) with Bu₄N⁺SCN⁻ (see i.e. J. Comb. Chem. 2001, 3, 90) or by rearrangement of the corresponding thiocyanato derivatives (IIe) (see Tetrahedron 1988, 44, 1619 or Synthesis 1986, 817).

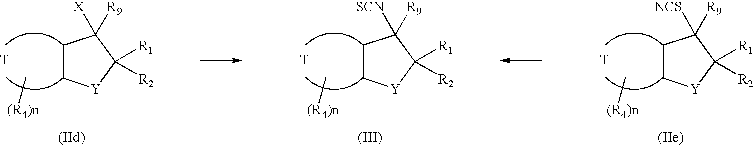

Amines of the formula (IIa), wherein R₉ is hydrogen, can be prepared in various ways. One method starts from ketones (IV). Transformation involves reduction of the ketone with NaBH₄/MeOH in the known manner and conversion to the corresponding azide according to WO 95/01970. Subsequent reduction with PPh₃/H₂O (e.g. J. Med. Chem. 2005, 48, 485) or in the presence of SnCl₂/MeOH (e.g. Synthetic Commun. 1991, 21, 733) leads to the desired amines (IIa).

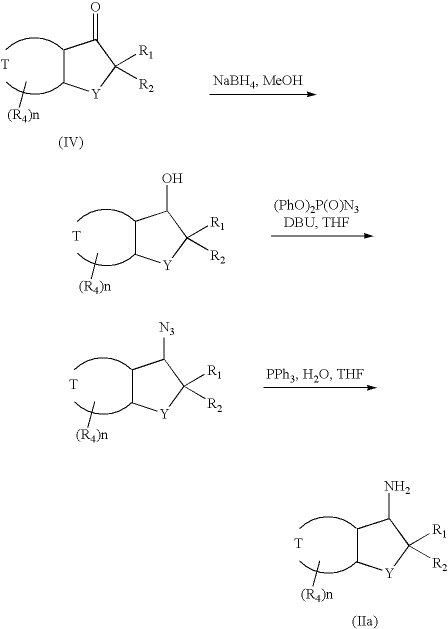

Amines of the formula (IIa) can also be prepared from carboxylic acids (V) by a Curtius degradation, e.g. using (PhO)₂P(O)N₃ as reagent (e.g. Tetrahedron Letters 1997, 38, 1681) as shown below.

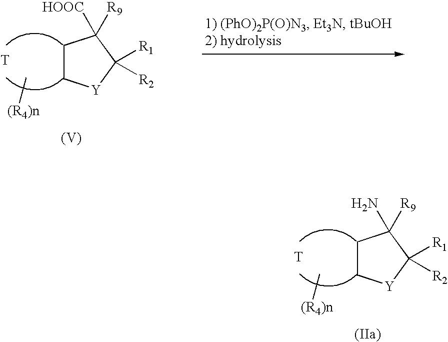

Amines of formula IIa, wherein Y is oxygen are partially known or may be prepared by the known methods as described in Chimica Acta Turica, 13(3), 403-412 (1985) and Farmaco, Edizione Scientifica, 43(7-8), 643-655 (1988).

The compounds of formula (I) can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fibre products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

Examples of pest species which may be controlled by the compounds of formula (I) include: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta migratoria (locust), Choffiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulphureus), Solenopsis geminata (fire ant), Monomorium pharaonis (pharaoh's ant), Damalinia spp. and Linognathus spp. (biting and sucking lice), and Deroceras reticulatum (slug).

The invention therefore provides a method of combating and controlling insects, acarines, or molluscs which comprises applying an insecticidally, acaricidally, nematicidally or molluscicidally effective amount of a compound of formula (I), or a composition containing a compound of formula (I), to a pest, a locus of pest, or to a plant susceptible to attack by a pest, The compounds of formula (I) are preferably used against insects or acarines.

The term “plant” as used herein includes seedlings, bushes and trees.

In order to apply a compound of formula (I) as an insecticide, acaricide, nematicide or molluscicide to a pest, a locus of pest, or to a plant susceptible to attack by a pest, a compound of formula (I) is usually formulated into a composition which includes, in addition to the compound of formula (I), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I). The composition is generally used for the control of pests such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal, acaricidal, nematicidal or molluscicidal composition comprising an insecticidally, acaricidally, nematicidally or molluscicidally effective amount of a compound of formula (I) and a suitable carrier or diluent therefor. The composition is preferably an insecticidal, acaricidal, nematicidal or molluscicidal composition.

In a still further aspect the invention provides a method of combating and controlling pests at a locus which comprises treating the pests or the locus of the pests with an insecticidally, acaricidally, nematicidally or molluscicidally effective amount of a composition comprising a compound of formula (I). The compounds of formula (I) are preferably used against insects, acarines or nematodes.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurised, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters), amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilisers (for example nitrogen-, potassium- or phosphorus-containing fertilisers). Suitable formulation types include granules of fertiliser. The mixtures suitably contain up to 25% by weight of the compound of formula (I).

The invention therefore also provides a fertiliser composition comprising a fertiliser and a compound of formula (I).

The compositions of this invention may contain other compounds having biological activity, for example micronutrients or compounds having fungicidal activity or which possess plant growth regulating, herbicidal, insecticidal, nematicidal or acaricidal activity.

The compound of formula (I) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;
b) Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron or chlorfluazuron;
e) Organic tin compounds, such as cyhexatin, fenbutatin oxide or azocyclotin;
f) Pyrazoles, such as tebufenpyrad and fenpyroximate;
g) Macrolides, such as avermectins or milbemycins, for example abamectin, emamectin benzoate, ivermectin, milbemycin, spinosad or azadirachtin;
h) Hormones or pheromones;
i) Organochlorine compounds such as endosulfan, benzene hexachloride, DDT, chlordane or dieldrin;
j) Amidines, such as chlordimeform or amitraz;
k) Fumigant agents, such as chloropicrin, dichloropropane, methyl bromide or metam;
l) Chloronicotinyl compounds such as imidacloprid, thiacloprid, acetamiprid, nitenpyram or thiamethoxam;
m) Diacylhydrazines, such as tebufenozide, chromafenozide or methoxyfenozide;
n) Diphenyl ethers, such as diofenolan or pyriproxifen;

### o) Indoxacarb;

### p) Chlorfenapyr; or

### q) Pymetrozine.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulphonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)-4-methylbenzamide (RH-7281, zoxamide), N-allyl-4,5-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5pyridyl)-cyclopropane carboxamide, acibenzolar (CGA245704), alanycarb, aldimorph, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulphate, copper tallate and Bordeaux mixture, cymoxanil, cyproconazole, cyprodinil, debacarb, di-2-pyridyl disulphide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl(Z)-N-benzyl-N([methyl(methyl-thioethylideneaminooxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, fluoroimide, fluquinconazole, flusilazole, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, mancozeb, maneb, mefenoxam, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, phenazin oxide, phosetyl-Al, phosphorus acids, phthalide, picoxystrobin (ZA1963), polyoxin D, polyram, probenazole, prochloraz, procymidone, propamocarb, propiconazole, propineb, propionic acid, pyrazophos, pyrifenox, pyrimethanil, pyroquilon, pyroxyfur, pyrrolnitrin, quaternary ammonium compounds, quinomethionate, quinoxyfen, quintozene, sipconazole (F-155), sodium pentachlorophenate, spiroxamine, streptomycin, sulphur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The invention is illustrated by the following Examples:

Mass spectra data were obtained for selected compounds of the following examples using LCMS: LC5: 254 nm—gradient 10% A to 100% B A=H₂O+0.01% HCOOH B═CH₃CN/CH₃OH+0.01% HCOOH positive electrospray 150-1000 m/z.

### EXAMPLE 1

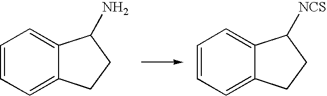

A mixture of 2.3 ml (27.1 mmol) thiophosgene in 20 ml dichloromethane is cooled to 0° C. after which a cold (0° C.) solution of 3.1 g (22.5 mmol) K₂CO₃ in 22 ml water is added. The mixture is stirred for 10 min and 3 ml (22.5 mmol) indan-1-ylamine is then added dropwise with vigorous stirring at 0° C. After an additional 10 min a cold solution of 2.5 g KOH in 22 ml water is added in one portion with cooling. The organic layer and three extracts (Et₂O) are combined, dried over MgSO₄, filtered and concentrated to give 3.9 g of crude 1-isothiocyanato-indane as a brown liquid. The product is used directly without further purification.

¹H-NMR (ppm, CDCl3): 2.20-2.30 (m, 1H); 2.50-2.60 (m, 1H); 2.85-2.95 (m, 1H); 3.05-3.15 (m, 1H); 5.20 (t, 1H); 7.20-7.35 (m, 3H); 7.40-7.50 (m, 1H).

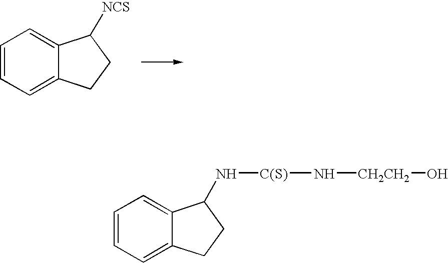

300 mg (1.7 mmol) 1-isothiocyanato-indane is dissolved in 6 ml THF at room temperature under argon. Then 0.103 ml (1.7 mmol) 2-amino-ethanol is added slowly. The reaction mixture is stirred for 2 hours at room temperature. THF is removed under reduced pressure to give the crude product, which is purified by column chromatography on silica gel, eluting with EtOAc/hexane (1:3). The title compound 1-(2-hydroxy-ethyl)-3-indan-1-yl-thiourea is obtained in 96% yield as a beige powder (Mp 119-121° C.).

¹H-NMR (ppm, DMSO): 1.70-1.80 (m, 1H); 2.40-2.50 (m, 1H); 2.75-2.85 (m, 1H); 2.85-2.95 (m, 1H); 3.50 (brs, 4H); 4.75 (brs, 1H); 5.8 (brs, 1H); 7.15-7.30 (m, 4H); 7.40 (brs, 1H); 7.85 (d, 1H).

### EXAMPLE 2

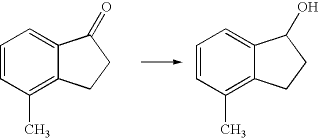

15.0 g (102.6 mmol) 4-methyl-indan-1-one is dissolved in 125 ml MeOH. The solution is cooled to 0-5° C. and 4.3 g (112.9 mmol) NaBH₄ is added portionwise while maintaining the temperature between 0-8° C. After the addition of NaBH₄ the cool-bath is removed and the reaction mixture is stirred till TLC indicates completion of the reaction. The reaction mixture is poured into water and extracted three times with EtOAc. The collected organic layers are washed with water and brine, dried over MgSO₄ and filtered. After evaporation of EtOAc 4-methyl-indan-1-ol is isolated in 98% yield. The crude material may be used directly without further purification.

¹H-NMR (ppm, CDCl₃): 1.95-2.03 (m, 1H); 2.30 (s, 3H); 2.45-2.55 (m, 1H); 2.70-2.80 (m, 1H); 2.95-3.05 (m, 1H); 5.25-5.30 (dd, 1H); 7.12 (d, 1H); 7.18 (t, 1H); 7.28 (d, 1H).

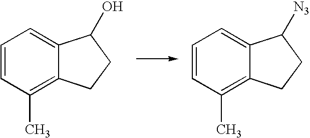

4.34 g (29.28 mmol) 4-methyl-indan-1-ol and 7.8 ml (35.14 mmol) diphenylphosphoryl azide are dissolved in dry THF at room temperature under argon. To this mixture is added slowly 5.0 ml (35.14 mmol) 1,8-diazabicyclo[5.4.0]undec-7-ene. The reaction is stirred for 6 h at room temperature (TLC monitoring). Then the mixture is poured into water and extracted three times with toluene. The organic layers are combined, dried over MgSO₄ and concentrated in vacuo. Purification using silica gel chromatography (hexane/EtOAc 9:1) affords pure 1-azido-4-methyl-indane in 80% yield as light orange liquid.

¹H-NMR (ppm, CDCl₃): 2.05-2.15 (m, 1H); 2.25 (s, 3H); 2.35-2.45 (m, 1H); 2.72-3.02 (m, 1H); 4.82-4.88 (dd, 1H); 7.10 (d, 1H); 7.15 (t, 1H); 7.22 (d, 1H).

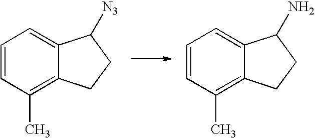

1.0 g (5.8 mmol) 1-azido-4-methyl-indane is treated with 4.54 g (17.3 mmol) triphenylphosphine in 26 ml THF and 1.4 ml H₂O at room temperature overnight. Completion of the reaction is achieved by heating up the mixture for additional 2.5 hours. The reaction mixture is then cooled to room temperature and diluted with water. The pH is adjusted to 2 by adding cold (ca 0° C.) aqueous HCl (1M). After extraction with EtOAc the aqueous layer is separated, basified with NaOH (2M) and extracted into EtOAc. The organic layer is then dried (MgSO₄) and concentrated in vacuo to give 0.61 g (72%) 4-methyl-indan-1-yl-amine as a light yellow liquid.

¹H-NMR (ppm, CDCl₃): 1.55 (brs, 1H); 1.60-1.70 (m, 1H); 2.25 (s, 3H); 2.45-2.52 (m, 1H); 2.62-2.72 (m, 1H); 2.85-2.94 (m, 1H); 4.33 (t, 1H); 6.98-7.05 (m, 1H); 7.10-7.15 (m, 2H).

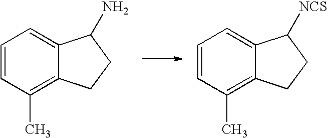

A mixture of 0.36 ml (4.32 mmol) thiophosgene in 4 ml dichloromethane is cooled to 0° C. after which a cold (0° C.) solution of 0.5 g (3.60 mmol) K₂CO₃ in 4 ml water is added. The mixture is stirred for 10 min and 0.53 g (3.60 mmol) indan-1-ylamine is then added drop wise with vigorous stirring at 0° C. After an additional 10 min a cold solution of 0.40 g (7.20 mmol) KOH in 4 ml water is added in one portion with cooling. The organic layer and three extracts (Et₂O) are combined, dried over MgSO₄, filtered and concentrated to give 0.68 g of crude 1-isothiocyanato-4-methyl-indane as an orange brown liquid. The product is used directly without further purification.

¹H-NMR (ppm, CDCl₃): 2.18-2.28 (m, 1H); 2.28 (s, 3H); 2.50-2.60 (m, 1H); 2.75-2.85 (m, 1H); 2.95-3.05 (m, 1H); 5.19 (t, 1H), 7.11 (d, 1H); 7.18 (t, 1H); 7.25 (d, 1H).

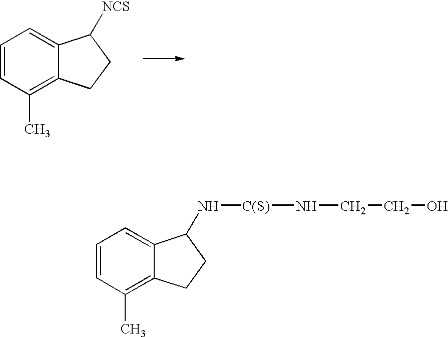

680 mg (3.59 mmol) 1-isothiocyanato-4-methyl-indane is dissolved in 13 ml THF at room temperature under argon. Then 0.21 ml (3.59 mmol) 2-amino-ethanol is added slowly. The reaction mixture is stirred at room temperature overnight. After completion of the reaction as indicated by TLC the solvent is removed under reduced pressure to give the crude product. Subsequent purification by column chromatography on silica gel (CH₂Cl₂/MeOH 15:1) and stirring in Et₂O affords the title compound 1-(2-hydroxy-ethyl)-3-(4-methyl-indan-1-yl)-thiourea in 82% yield as a white powder (Mp 90-93° C.).

¹H-NMR (ppm, CDCl₃): 1.85-1.95 (m, 1H); 2.25 (s, 3H); 2.55 (br s, 1H); 2.60-2.70 (m, 1H); 2.71-2.81 (m, 1H); 2.86-2.99 (m, 1H); 3.62 (br s, 2H); 3.78 (t, 2H); 5.67 (br s, 1H); 6.47 (br s, 1H), 6.11 (br s, 1H); 7.07 (d, 1H); 7.12 (t, 1H); 7.18 (d, 1H).

### EXAMPLE 3

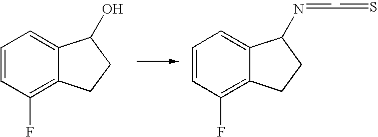

A mixture of 0.304 g (2 mmol) 4-fluoro-indan-1-ol and 0.233 g (2.4 mmol) potassium rhodanide were heated in the presence of 0.180 g (2 mmol) oxalic acid, 0.400 g (2.4 mmol) potassium iodide and 0.051 (0.4 mmol) iodine in 5 ml nitromethane to 90° C. and held over night. The cold reaction mixture was then filtered through Hyflo®, extracted with ethylacetate against water and brine and evaporated. The crude material was purified by silica chromatography (eluent ethyl acetate/n-heptane 1:9) and almost pure 4-fluoro-1-isothiocyanato-indane was obtained.

¹H-NMR (ppm, CDCl₃): 2.23-2.33 (m, 1H); 2.55-2.65 (m, 1H); 2.85-2.95 (m, 1H); 3.08-3.18 (m, 1H); 5.19 (t, 1H); 7.00 (m, 1H); 7.15-7.30 (m, 2H).

### EXAMPLE 4

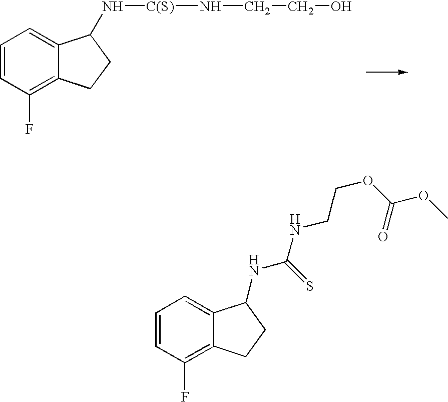

509 mg (2.0 mmol) 1-(2-hydroxy-ethyl)-3-(4-fluoro-indan-1-yl)-thiourea and 646 mg (5.0 mmol) Hünig's base (N,N′-diisopropylethylamine) are dissolved in 10 ml THF at room temperature. Then 0.36 ml (5.0 mmol) acetyl chloride is added dropwise keeping the temperature below 20° C. The reaction mixture is then stirred at room temperature overnight. After completion of the reaction (indicated by TLC) the mixture is diluted with water. After extraction with EtOAc the organic layer is separated, washed (H₂O) and dried over Na₂SO₄. The crude material is purified by silica chromatography (eluent ethylacetate/n-heptane 1:7 at p=1.3 bar) and 135 mg carbonic acid 2-[3(4-fluoro-indan-1-yl)-thioureido]-ethyl methylester are obtained (Mp.: 137-138° C.).

¹H-NMR (ppm, CDCl₃): 1.95-2.05 (m, 1H); 2.07 (s, 3H); 2.68-2.78 (m, 1H); 2.88-2.2.98 (m, 1H); 3.05-3.16 (m, 1H); 3.80 (br t, 2H); 4.29 (t, 2H); 5.71 (brs, 1H); 6.98 (t, 1H); 7.15-7.25 (m, 2H).

As a byproduct 65 mg of the corresponding bis acetyl compound (=compound 10.032) were isolated as a colourless oil. ¹H-NMR data of carbonic acid 2-[1-acetyl-3-(4-fluoro-indan-1-yl)-thioureido]-ethyl methylester is shown below:

¹H-NMR (ppm, CDCl₃): 1.86-1.98 (m, 1H); 2.0 (s, 3H); 2.48 (s; 3H); 2.69-2.88 (m, 2H); 2.95-3.05 (m, 1H); 4.35 (t, 2H), 4.55 (t, 2H); 5.90 (q, 1H); 6.82-6.91 (m, 1H); 7.07-7.15 (m, 2H); 11.5 (s, 1H).

A further product (compound=10.033) obtained is carbonic acid 2-[3-acetyl]-3-(4-fluoro-indan-1-yl)-thioureido]-ethyl methylester (355 mg).

¹H-NMR (ppm, CDCl₃): 2.05 (s, 3H); 2.1 (s, 3H); 22.30-2.42 (m, 1H); 2.6-2.75 (m, 1H); 2.38-3.0 (m, 1H), 2.85-3.0 (m, 1H); 3.1-3.22 (m, 1H); 3.75-3.92 (m, 2H); 4.12-4.28 (m, 2H); 6.65 (t, 1H); 6.92 (t, 1H); 7.05 (d, 1H); 7.15-7.22 (m, 1H).

### EXAMPLE 5

This Example illustrates the pesticidal/insecticidal properties of compounds of formula (I). Test against were performed as follows:

### Heliothis Virescens (Tobacco Budworm):

Eggs (0-24 h old) were placed in 24-well microtiter plate on artificial diet and treated with test solutions at an application rate of 200 ppm by pipetting. After an incubation period of 4 days, samples were checked for egg mortality, larval mortality, and growth regulation.
The following compounds gave at least 80% control of Heliothis virescens:
1.050; 1.073; 1.088; 3.082; 11.023; 11.031; 11.034; 11.046; 11.051.

### Myzus Persicae (Green Peach Aphid):

Sunflower leaf discs were placed on agar in a 24-well microtiter plate and sprayed with test solutions at an application rate of 200 ppm. After drying, the leaf discs were infested with an aphid population of mixed ages. After an incubation period of 6 DAT, samples were checked for mortality.
The following compounds gave at least 80% control of Myzus persicae:
1.001; 1.012; 1.027; 1.050, 1.073, 1.088; 1.092; 1.118; 1.205; 1.422; 1.424; 1.425; 2.091; 2.201; 3.004; 3.010; 3.020; 3.031; 3.035; 3.038; 3.040; 3.043; 3.046; 3.064; 3.065; 3.066; 3.067; 3.068; 3.069; 3.070; 3.071; 3.072; 3.073; 3.074; 3.076; 3.080; 3.081; 3.082; 3.083; 3.084; 3.086; 3.087; 3.089; 3.090; 3.094; 3.095; 3.096; 3.097; 3.099; 3.100; 3.101; 3.102; 3.103; 3.104; 3.105; 3.106; 3.107; 3.108; 3.110; 3.111; 3.112; 3.113; 3.115; 3.116; 3.117; 4.003; 6.001; 8.002; 9.001; 10.007; 10.009; 10.010; 10.014; 10.015; 10.016; 10.017; 10.018; 10.019; 10.020; 10.021; 10.022; 10.024; 10.025; 10.026; 10.027; 10.028; 10.030; 10.032; 10.033; 10.034; 11.001; 11.003; 11.005; 11.006; 11.007; 11.009; 11.010; 11.011; 11.012; 11.013; 11.014; 11.015; 11.016; 11.018; 11.023; 11.024; 11.027; 11.028; 11.030; 11.031; 11.034; 11.035; 11.039; 11.040; 11.041; 11.043; 11.044; 11.045; 11.046; 11.047; 11.048; 11.049; 11.051; 11.052; 11.053; 11.054; 11.055; 11.058; 11.059; 11.060; 11.062; 11.063; 11.064; 11.065; 11.067; 11.068; 11.070; 11.072.

### Myzus Persicae (Green Peach Aphid):

Roots of pea seedlings, infested with an aphid population of mixed ages, were placed directly in the test solutions of 24 ppm. 6 days after introduction, samples were checked for mortality.
The following compounds gave at least 80% control of Myzus persicae:
1.001, 1.012; 1.027; 1.050, 1.073, 1.088; 1.092; 1.118; 1.422; 1.424; 1.425; 3.004; 3.010; 3.020; 3.031; 3.035; 3.038; 3.040; 3.043; 3.046; 3.064; 3.065; 3.066 3.067; 3.068; 3.069; 3.070; 3.071; 3.072; 3.073; 3.074; 3.076; 3.080; 3.081; 3.082; 3.083; 3.084; 3.086; 3.087; 3.089; 3.090; 3.094; 3.095; 3.096; 3.097; 3.099; 3.100; 3.101; 3.102; 3.103; 3.104; 3.105; 3.106; 3.107; 3.110; 3.112; 3.113; 3.116; 3.117; 10.007; 10.009; 10.010; 10.014; 10.017; 10.018; 10.019; 10.020; 10.021; 10.022; 10.030; 10.032; 11.003; 11.005; 11.006; 11.007; 11.009; 11.010; 11.011; 11.012; 11.013; 11.014; 11.016; 11.018; 11.023; 11.024; 11.028; 11.030; 11.031; 11.034; 11.035; 11.040; 11.044; 11.045; 11.047; 11.048; 11.049; 11.051; 11.053; 11.054; 11.055; 11.058; 11.059; 11.060; 11.062; 11.063; 11.065; 11.070.

### Tetratnychus Urticae (Two-Spotted Spider Mite):

Bean leaf discs on agar in 24-well microtiter plates were sprayed with test solutions at an application rate of 200 ppm. After drying, the leaf discs are infested with mite populations of mixed ages. 8 days later, discs are checked for egg mortality, larval mortality, and adult mortality.
The following compounds gave at least 80% control of Tetranychus urticae:
1.050, 1.073, 1.088; 1.092; 1.422; 1.424; 1.425; 3.004; 3.020; 3.031; 3.038; 3.040; 3.043; 3.046; 3.066; 3.069; 3.072; 3.074; 3.080; 3.081; 3.082; 3.084; 3.086; 3.087; 3.089; 3.090; 3.095; 3.096; 3.097; 3.099; 3.100; 3.101; 3.102; 3.103; 3.104; 3.105; 3.106; 3.107; 3.108; 3.110; 3.111; 3.112; 3.113; 3.115; 3.116; 3.117; 10.007; 10.009; 10.010; 10.012; 10.017; 10.018; 10.019; 10.024; 10.025; 10.028; 10.030; 10.032; 11.001; 11.003; 11.005; 11.006; 11.007; 11.009; 11.010; 11.011; 11.012; 11.013; 11.014; 11.016; 11.018; 11.023; 11.024; 11.028; 11.030; 11.035; 11.040; 11.041; 11.043; 11.044; 11.046; 11.047; 11.048; 11.051; 11.052; 11.053; 11.055; 11.058; 11.059; 11.060; 11.062; 11.063; 11.064; 11.065; 11.067; 11.068; 11.070; 11.072.

1. A method of combating and controlling insects, acarines, or molluscs which comprises applying to a pest, to a locus of a pest, or to a plant susceptible to attack by a pest an insecticidally, acaricidally, nematicidally or molluscicidally effective amount of a compound of formula (I): 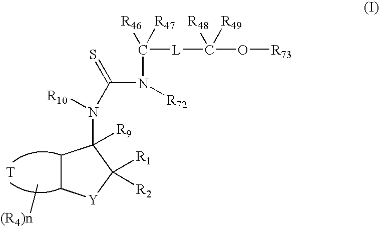
wherein
R⁷³ is hydrogen, G-, formyl, G-C(O)—, G-C(S)—, G-O—C(O)—, G-O—C(S)—, R⁷⁸R⁷⁹N—C(O)—, R⁷⁸R⁷⁹N—C(S)—, where R⁷⁸ and R⁷⁹ are independently H or G-, or R⁷⁸ and R⁷⁹ together with the N atom to which they are attached, form a group N═CRaRb where Ra and Rb are H, C₁₋₆ alkyl or phenyl; or R⁷⁸ and R⁷⁹ together with the N atom to which they are attached form a five, six or seven membered ring which may contain one or two further hetero atoms selected from O, N or S and which may be optionally substituted by one to four C₁₋₆ alkyl groups;
R¹⁰ and R⁷² are independently hydrogen, hydroxy, amino, cyano, formyl, G-, G-O—, G-S—, G-S—S—, G-A-, R²⁴R²⁵N—, G-A-NR¹⁷, R²⁴R²⁵N S—, R²⁴R²⁵N-A R¹⁸N═C(R¹⁹)—, G-O-A- or G-S-A-; where R²⁴ and R²⁵ are independently H or G-, or R²⁴ and R²⁵ together with the N atom to which they are attached, form a group N═CRaRb where Ra and Rb are H, C₁₋₆ alkyl or phenyl; or R²⁴ and R²⁵ together with the N atom to which they are attached form a five, six or seven membered ring which may contain one or two further hetero atoms selected from O, N or S and which may be optionally substituted by one to four C₁₋₆ alkyl groups or phenyl; R¹⁷ is H, G-, G-C(O)— or G-OC(O)—; R¹⁸ is H, OH, cyano, nitro, G-, G-O— or R³⁸R³⁹N—, where R³⁸ and R³⁹ are independently H or G-, or R³⁸ and R³⁹ together with the N atom to which they are attached, form a group N═CRaRb where Ra and Rb are H, C₁₋₆ alkyl or phenyl; or R³⁸ and R³⁹ together with the N atom to which they are attached form a five, six or seven membered ring which may contain one or two further hetero atoms selected from O, N or S and which may be optionally substituted by one to four C₁₋₆ alkyl groups; R¹⁹ is H, cyano, G-, G-O—, G-S— or R⁴²R⁴³N—, where R⁴² and R⁴³ are independently H or G-; or R⁴² and R⁴³ together with the N atom to which they are attached, form a group N═CRaRb where Ra and Rb are H, C₁₋₆ alkyl or phenyl; or R⁴² and R⁴³ together with the N atom to which they are attached form a five, six or seven membered ring which may contain one or two further hetero atoms selected from O, N or S and which may be optionally substituted by one to four C₁₋₆ alkyl groups or phenyl;
L is a direct bond, CR⁷⁴R⁷⁵ or CR⁷⁴R⁷⁵—CR⁷⁶, R⁷⁷, where R⁷⁴, R⁷⁵, R⁷⁶ and R⁷⁷ are each independently hydrogen, OH, halogen, COOH, cyano, formyl, G-, GO-, GS—, G-C(O)—, G-C(S)—, G-O—C(O)—, G-O—C(S)—, R⁸⁰R⁸¹N C(O)—, R⁸⁰R⁸¹N—C(S)—; or the groups R⁷⁴ and R⁷⁵ and/or R⁷⁶ and R⁷⁷ together with the carbon atom to which they are attached form a three to six membered ring, containing at least 2 carbon atoms and optionally containing one or two sulphur and/or one or two non-adjacent oxygen atoms or a group NR⁸², S(O)₂, S(O), or C(O) the ring being optionally substituted by C₁₋₆ alkyl or phenyl; where R⁸⁰ and R⁸¹ are independently H or G-, or R⁸⁰ and R⁸¹ together with the N atom to which they are attached, form a group N═CRaRb where Ra and Rb are H, C₁₋₆ alkyl or phenyl; or R⁸⁰ and R⁸¹ together with the N atom to which they are attached form a five, six or seven membered ring which may contain one or two further hetero atoms selected from O, N or S and which may be optionally substituted by one to four C₁₋₆alkyl groups; R⁸² is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷ and R²³ are independently H or G-, or R²⁷ and R²³ together with the N atom to which they are attached, form a group N═CRaRb where Ra and Rb are H, C₁₋₆ alkyl or phenyl; or R²⁷ and R²⁸ together with the N atom to which they are attached form a five, six or seven membered ring which may contain one or two further hetero atoms selected from O, N or S and which may be optionally substituted by one to four C₁₋₆ alkyl groups or phenyl; R²⁹ is H or G-; or two of the groups R⁷⁴, R⁷⁵, R⁷⁶ and R⁷⁷ attached to different atoms together with the atoms to which they are attached form a three to seven membered ring, that optionally contains one or two sulphur and/or one or two non-adjacent oxygen atoms or a group NR⁸², S(O)₂, S(O) or C(O), the ring being optionally substituted by C₁-C₆ alkyl or phenyl; where R⁸² has the meanings assigned to it above;
R⁴⁶, R⁴⁷, R⁴⁸ and R⁴⁹ are each independently hydrogen, halogen, G-, G-C(O)—, G-C(S)—, G-O—C(O)—, G-O—C(S)—, R⁸³R⁸⁴N—C(O)—, R⁸³R⁸⁴N—C(S)—; where R⁸³ and R⁸⁴ are independently H or G-, or R³³ and R³⁴ together with the N atom to which they are attached, form a group N═CRaRb where Ra and Rb are H, C₁₋₆ alkyl or phenyl; or R⁸³ and R⁸⁴ together with the N atom to which they are attached form a five, six or seven membered ring which may contain one or two further hetero atoms selected from O, N or S and which may be optionally substituted by one to four C₁₋₆ alkyl groups or phenyl, or the groups R⁴⁶ and R⁴⁷ and/or R⁴⁸ and R⁴⁹ together with the carbon atom to which they are attached form a three to six membered ring, containing at least 2 carbon atoms and optionally containing one or two sulphur and/or one or two non-adjacent oxygen atoms or a group NR⁸⁵, where R⁸⁵ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹, where R²⁷ and R²⁸ are independently H or G-, or R²⁷ and R²⁸ together with the N atom to which they are attached, form a group N═CRaRb where Ra and Rb are H, C₁₋₆ alkyl or phenyl; or R²⁷ and R²³ together with the N atom to which they are attached form a five, six or seven membered ring which may contain one or two further hetero atoms selected from O, N or S and which may be optionally substituted by one to four C₁₋₆ alkyl groups or phenyl; or two of the groups R⁴⁶, R⁴⁷, R⁴⁸, R⁴⁹, R⁷⁴, R⁷⁵, R⁷⁶ and R⁷⁷ attached to different atoms form together with the atoms to which they are attached a three to seven membered ring, that optionally contains one or two sulphur and/or one or two non-adjacent oxygen atoms or a group NR⁸⁵, where R⁸² and R⁸⁵ have the meanings assigned to them as above, or a group S(O)₂, S(O) or C(O) the ring being optionally substituted by C₁-C₆ alkyl or phenyl;
Y is O, S(O)ₘ, where m is 0, 1 or 2, NR³, SO₂—NR³, NR³—SO₂, NR³—O or O—NR³ where R³ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²³ and R²⁹ have the meanings assigned to them above, or Y is CR⁵R⁵, CR⁵R⁶—CR⁷R⁸, O—CR⁷R⁸, S(O)ₘ—CR⁷R⁸, NR³—CR⁷R³, CR⁵R⁶—O, CR⁵R⁶—S(O)ₘ, CR⁵R⁵—NR³, where R³ and m have the meanings assigned to them above, and R⁵, R⁶, R⁷ and R⁸ are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²²N-A-O—, R²¹R²²N-A-S—, R²¹R²²N-A-NR²³—, G-O-A-O—, G-O-A-S—, G-O-A-NR²³—, G-S-A-O, G-S-A-NR²³—, or R²⁰S(O)(═NR¹⁷)—, where R²¹ and R²² are independently H or G-, or R²¹ and R²² together with the N atom to which they are attached, form a group N═CRaRb where Ra and Rb are H, C₁₋₆ alkyl or phenyl; or R²¹ and R²² together with the N atom to which they are attached form a five, six or seven membered ring which may contain one or two further hetero atoms selected from O, N or S and which may be optionally substituted by one to four C₁₋₆ alkyl groups or phenyl; R²³ is H or G- and R¹⁷ is as defined above; R²⁰ is C₁₋₆ alkyl, optionally substituted phenyl, optionally substituted benzyl; or two of the groups R⁵, R⁶, R⁷ and R⁸ attached to the same carbon atom are ═O, ═S, ═NR¹¹ or ═CR¹²R¹³, where R¹¹ is H, OH, nitro, cyano, formyl, formyloxy, G-, G-O—, G-A-, R³⁶R³⁷N—, G-C(O)—O—, G-C(O)—NR²⁶—, R³⁶, R³⁷N—C(O)O—, G-O—C(O)O—, G-O—C(O)—NR²⁶—, where R³⁶, R³⁷ and R²⁵ are independently H or G-, or R³⁶ and R³⁷ together with the N atom to which they are attached, form a group N═CRaRb where Ra and Rb are H, C₁₋₆ alkyl or phenyl; or R³⁶ and R³⁷ together with the N atom to which they are attached form a five, six or seven membered ring which may contain one or two further hetero atoms selected from O, N or S and which may be optionally substituted by one to four C₁₋₆ alkyl groups or phenyl, and R¹² and R¹³ are each independently H, halogen, nitro, cyano, formyl, formyloxy, G-, G-O—, G-S—, G-A-, R⁴⁰R⁴¹N—, R⁴⁰R⁴¹N-A-, G-O-A-, G-A-O—, R⁴⁰R⁴¹N-A-O—, R⁴⁰R⁴¹N-A-S—, G-O-A-O—, G-O-A-S—, G-O-A-NR³⁰—, where R⁴⁰, R⁴¹ and R³⁰ are independently H or G-, or R⁴⁰ and R⁴¹ together with the N atom to which they are attached, form a group N═CRaRb where Ra and Rb are H, C₁₋₆ alkyl or phenyl; or R⁴⁰ and R⁴¹ together with the N atom to which they are attached form a five, six or seven membered ring which may contain one or two further hetero atoms selected from O, N or S and which may be optionally substituted by one to four C₁₋₆ alkyl groups or phenyl, or R¹² and R¹³ together with the carbon atom to which they are attached form a 3 to 6 membered carbocyclic ring; or the groups R⁵ and R⁶ or R⁷ and R⁸ together with the carbon atom to which they are attached form a three to six membered ring, containing at least 2 carbon atoms and optionally containing one or two sulfur and/or one or two non-adjacent oxygen atoms or a group NR¹⁴, where R¹⁴ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²⁸ and R²⁹ have the meanings assigned to them above, the ring being optionally substituted by one to four C₁-C₆alkyl groups or phenyl; or two of the groups R⁵, R⁶, R⁷ and R³ attached to different atoms together with the atoms they are attached form a three to seven membered ring, that optionally contains one or two sulfur and/or one or two non-adjacent oxygen atoms or a group NR¹⁴, where R¹⁴ is as defined above, or two of the groups R⁵, R⁶, R⁷ and R⁸ attached to adjacent atoms combine to form a bond;
the ring (T) 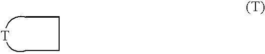
is a 5- or 6-membered aromatic or heteroaromatic ring;
R¹ and R² are each independently H, OH, halogen, nitro, cyano, rhodano, carboxy, formyl, formyloxy, G-, G-O—, G-S—, G-A-, R²¹R²²N—, R²¹R²²N-A-, G-O-A-, G-S-A-, G-A-O—, G-A-S—, G-A-NR²³—, R²¹R²² N-A-O—, R²¹R²²N-A-S—, R²¹R²²N-A-NR²³—, G-O-A-O—, G-O-A-S—, G-O-A-NR²³—, G-S-A-O, G-S-A-NR²³—, or R²⁰S(O)(═NR¹⁷)—, where R¹⁷, R²⁰, R²¹, R²² and R²³ are as defined above, or two of the groups R¹ and R² attached to the same carbon atom are ═O, ═S, ═NR¹¹ or ═CR¹²R¹³, where R¹¹, R¹² and R¹³ are defined as above, or the groups R¹ and R² together with the same carbon atom to which they are attached form a three to six membered ring, containing at least 2 carbon atoms and optionally containing one or two sulfur and/or one or two non-adjacent oxygen atoms or a group NR¹⁴, where R¹⁴ is as defined above, the ring being optionally substituted by C₁-C₆ alkyl; or two of the groups R¹, R² and R⁷, R⁸ attached to different atoms together with the atoms they are attached form a three to seven membered ring, that optionally contains one or two sulfur and/or one or two non-adjacent oxygen atoms or a group NR¹⁴, where R¹⁴ is defined as above, the ring being optionally substituted by one or four C₁-C₆alkyl groups or phenyl; or two of the groups R¹, R², R⁵, R⁶, R⁷ and R³ attached to adjacent atoms combine to form a bond; each R⁴ is independently OH, halogen, nitro, cyano, azido, rhodano, isothiocyanato, carboxy, formyl, formyloxy, G-, G-O—, G-S—, G-A-, R³¹R³²N—, R³¹R³²N-A-, G-O-A-, G-S-A-, G-A-O—, G-A-S—, G-A-NR³³—, R³¹R³²N-A-O—, R³¹R³²N-A-S—, R³¹R³²N-A-NR³³—, G-O-A-O—, G-O-A-S—, G-O-A-NR³³—, G-S-A-O, G-S-A-NR³³—, R²⁰S(O)(═NR¹⁷)—, R¹⁸N═C(R¹⁹)—, R⁴⁴R⁴⁵P(O)— or R⁴⁴R⁴⁵P(S)—, where R¹⁷, R¹⁸, R¹⁹ and R²⁰ have the meanings assigned to them above, and R³¹, R³² and R³³ are independently H or G-, or R³¹ and R³² together with the N atom to which they are attached, form a group N═CRaRb where Ra and Rb are H, C₁₋₆ alkyl or phenyl; or R³¹ and R³² together with the N atom to which they are attached form a five, six or seven membered ring which may contain one or two further hetero atoms selected from O, N or S and which may be optionally substituted by one to four C₁₋₆ alkyl groups or phenyl, and R⁴⁴ and R⁴⁵ are independently H, C₁₋₆alkyl, C₁₋₆-alkoxy, phenyl, phenoxy; or 2 adjacent groups R⁴ together with the carbon atoms to which they are attached form a 4, 5, 6 or 7 membered carbocyclic or heterocyclic ring which may be optionally substituted by C₁₋₆alkyl or halogen; or a group R⁴ together with a group R³, R⁵, R⁶ or R⁹ and the atoms to which they are attached form a 5-7 membered ring optionally containing an NR¹⁵group where R¹⁵ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²³ and R²⁹ have the meanings assigned to them above, or containing an S or O atom, the ring being optionally substituted by one to four C₁-C₆ alkyl groups or phenyl;
n is 0, 1, 2, 3 or 4;
R⁹ is H, formyl, G-, G-A-, R³⁴R³⁵N-A-, where R³⁴ and R³⁵ are independently H or G-, or R³⁴ and R³⁵ together with the N atom to which they are attached, form a group N═CRaRb where Ra and Rb are H, C₁₋₆ alkyl or phenyl; or R³⁴ and R³⁵ together with the N atom to which they are attached form a five, six or seven membered ring which may contain one or two further hetero atoms selected from O, N or S and which may be optionally substituted by one to four C₁₋₆ alkyl groups or phenyl; or R⁹ is G-O-A- or G-S-A-; or R⁹ together with a group R¹, R², R³, R⁵, R⁶, R⁷ or R³ and the atoms to which they are attached may form a three to seven membered ring, that optionally may contain one or two sulfur and/or one or two non-adjacent oxygen atoms or a group NR¹⁶, where R¹⁶ is H, OH, cyano, formyl, G-, G-O—, G-S—, G-A-, R²⁷R²⁸N—, R²⁷R²⁸N-A-, G-O-A-, G-S-A-, G-A-NR²⁹—, R²⁷R²⁸N-A-NR²⁹—, G-O-A-NR²⁹— or G-S-A-NR²⁹—, where R²⁷, R²³ and R²⁹ have the meanings assigned to them above;
G is optionally substituted C₁₋₁₂ alkyl, optionally substituted C₂₋₁₂ alkenyl, optionally substituted C₂₋₁₂ alkynyl, optionally substituted C₃₋₈ cycloalkyl, optionally substituted C₃₋₈ cycloalkenyl, optionally substituted aryl, optionally substituted heteroaryl or optionally substituted heterocyclyl;
A is S(O), SO₂, C(O) or C(S);
or salts or N-oxides thereof,
with the proviso, that the compound of formula I is not: 1-(2-hydroxy-ethyl)-3-(5-methoxy-1,2,3,4-tetrahydro-naphthalen-1-yl)-thiourea, 1-(2-hydroxy-ethyl)-3-(1,2,3,4-tetrahydro-naphthalen-1-yl)-thiourea, methoxy-acetic acid 2-[3-(1,2,3,4-tetrahydro-naphthalen-1-yl)-thioureido]-ethyl ester, acetic acid 2-[3-(7-chloro-1,2,3,4-tetrahydro-naphthalen-1-yl)-thioureido]-ethyl ester, benzoic acid 2-[3-(7-chloro-1,2,3,4-tetrahydro-naphthalen-1-yl)-thioureido]-ethyl ester, acetic acid 2-[3-(7-bromo-1,2,3,4-tetrahydro-naphthalen-1-yl)-thioureido]-ethyl ester, benzoic acid 2-[3-(5-methoxy-1,2,3,4-tetrahydro-naphthalen-1-yl)-thioureido]-ethyl ester, 1-(7-bromo-1,2,3,4-tetrahydro-naphthalen-1-yl)-3-(2-hydroxy-ethyl)-thiourea, 1S-benzoic acid 2-(3-indan-1-yl-thioureido)-ethyl ester, nicotinic acid 2-(3-indan-1-yl-thioureido)-ethyl ester, nicotinic acid 2-(1-acetyl-3-indan-1-yl-thioureido)-ethyl ester, isonicotinic acid 2-(3-indan-1-yl-thioureido)-ethyl ester, pyridine-2-carboxylic acid 2-(3-indan-1-yl-thioureido)-ethyl ester, methoxy-acetic acid 2-(3-indan-1-yl-thioureido)-ethyl ester, 1-(2-hydroxy-ethyl)-3-indan-1-yl-thiourea, 1R-benzoic acid 2-(3-indan-1-yl-thioureido)-ethyl ester, acetic acid 2-[3-(6-methoxy-indan-1-yl)-thioureido]-ethyl ester, acetic acid 2-[3-(4-chloro-indan-1-yl)-thioureido]-ethyl ester, benzoic acid 2-[3-(4-chloro-indan-1-yl)-thioureido]-ethyl ester, 1-indan-1-yl-3-[2-(thiazol-2-yloxy)-ethyl]-thiourea, 4,5-dihydro-thiazole-2-carboxylic acid 2-(3-indan-1-yl-thioureido)-ethyl ester, 5-chloro-thiophene-2-carboxylic acid 2-((S)-3-indan-1-ylthioureido)-ethyl ester, furan-2-carboxylic acid 2-((S)-3-indan-1-ylthioureido)-ethyl ester, 3-trifluoromethyl-benzoic acid 2-((S)-3-indan-1-ylthioureido)-ethyl ester, 2-ethylsulfanyl-nicotinic acid 2-((S)-3-indan-1-ylthioureido)-ethyl ester, 2-methylsulfanyl-nicotinic acid 2-((S)-3-indan-1-ylthioureido)-ethyl ester, 3-cyano-benzoic acid 2-((S)-3-indan-1-ylthioureido)-ethyl ester, phenyl-acetic acid 2-((S)-3-indan-1-ylthioureido)-ethyl ester, 2-acetoxy-benzoic acid 2-((S)-3-indan-1-ylthioureido)-ethyl ester, diphenyl-acetic acid 2-((S)-3-indan-1-ylthioureido)-ethyl ester, 3-(2-chloro-phenyl)-5-methyl-isoxazole-4-carboxylic acid 2-((S)-3-indan-1-ylthioureido)-ethyl ester, 2-phenyl-butyric acid 2-((S)-3-indan-1-ylthioureido)-ethyl ester, cyclopentanecarboxylic acid 2-((S)-3-indan-1-ylthioureido)-ethyl ester, 3-methyl-butyric acid 2-((S)-3-indan-1-ylthioureido)-ethyl ester, naphthalene-2-carboxylic acid 2-((S)-3-indan-1-ylthioureido)-ethyl ester, 2,2-dimethyl-propionic acid 2-((S)-3-indan-1-ylthioureido)-ethyl ester, 3-phenyl-propionic acid 2-((S)-3-indan-1-ylthioureido)-ethyl ester, thiophen-2-yl-acetic acid 2-((S)-3-indan-1-ylthioureido)-ethyl ester, propionic acid 2-((S)-3-indan-1-ylthioureido)-ethyl ester, 3-methylsulfanyl-propionic acid 2-((S)-3-indan-1-ylthioureido)-ethyl ester, phenoxy-acetic acid 2-((S)-3-indan-1-ylthioureido)-ethyl ester, or 3,5-dimethyl-isoxazole-4-carboxylic acid 2-((S)-3-indan-1-ylthioureido)-ethyl ester.

2. The method according to claim 1, wherein the compound of formula I is a compound of formula (IA) 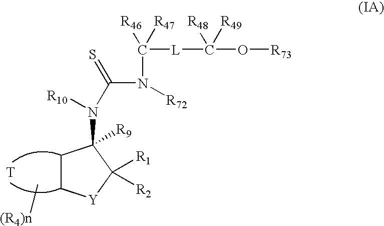
wherein the chirality on the carbon atom bearing R⁹ is as shown, and L, T, Y, R¹, R², R⁴, R⁹, R¹⁰, R⁴⁶, R⁴⁷, R⁴⁸, R⁴⁹, R⁷², R⁷³ and n are as defined in claim 1.

3. The method according to claim 1, wherein the ring (T) 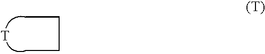
is an aromatic ring, n is 1, 2 or 3, and at least one substituent R⁴ is selected from fluoro, methyl, fluoromethyl, difluoromethyl, or trifluoromethyl.

4. The method according to claim 1, wherein the ring (T) 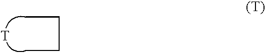
is a 5- or 6-membered heteroaromatic ring, wherein the ring members are each independently CH, S, N, NR⁴, O or CR⁴ and R⁴ is as defined in claim 1, provided that there is no more than one O or S atom present in the ring.

5. The method according to claim 1, wherein Y is O, S(O)ₘ, NR³, SO₂—NR³, NR³—SO₂, NR³—O, O—NR³, O—CR⁷R⁸, S(O)ₘ—CR⁷R⁸, NR³—CR⁷R⁸, CR⁵R⁶—O, CR⁵R⁶—S(O)ₘ or CR⁵R⁶—NR³, and wherein R³, R⁵, R⁶, R⁷ and R³ are as defined in claim 1.

6. The method according to claim 1, wherein R¹ and R² are each independently: hydrogen; hydroxyl; halogen; cyano; C₁₋₆ alkyl; C₁₋₆ haloalkyl; C₁₋₆ cyanoalkyl; C₁₋₆ hydroxyalkyl; C₁₋₆ alkoxy(C₁₋₆)alkyl; phenyl(C₁₋₃)alkyl, wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, or C₁₋₆ alkoxycarbonyl; C₃₋₅ cycloalkyl; 1,3-dioxolan-2-yl; phenyl optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino, dialkylamino, C₁₋₆ alkylsulfonyl, or C₁₋₆ alkoxycarbonyl; C₁₋₆ alkoxy; C₁₋₆ haloalkoxy; C₂₋₆ alkenyloxy; C₂₋₆ alkynyloxy; C₁₋₆ alkylthio; C₁₋₆ haloalkylthio; formyl; C₂₋₆ alkylcarbonyl; phenylcarbonyl wherein the phenyl group is optionally substituted by halogen, C₁₋₄ alkyl, C₁₋₄ alkoxy, C₁₋₄ haloalkyl, C₁₋₄ haloalkoxy, CN, or NO₂; or R¹ and R² together are ═O, ═S, ═NR¹¹ or ═CR¹²R¹³, wherein R¹¹ is OH, C₁₋₆ alkoxy or C₁₋₆ alkylcarbonylamino, and R¹² and R¹³ are each independently H, C₁₋₆ alkyl, or C₁₋₆ haloalkyl; or R¹ and R⁹ together with the carbon atom to which they are attached form a three to six membered ring, wherein said ring optionally comprises one, or two non-adjacent, oxygen atom(s); or R¹ and R² together with the carbon atom to which they are attached form a three to six membered ring, wherein said ring optionally comprises, one, or two non-adjacent, oxygen atom(s).

7. The method according to claim 1, wherein n is 1, 2 or 3 and each R⁴ is independently: halogen; cyano; C₁₋₈ alkyl; C₁₋₈ haloalkyl; cyano(C₁₋₆)alkyl; C₁₋₃ alkoxy(C₁₋₃)alkyl; C₂₋₆ alkynyl; C₃₋₆ cycloalkyl; C₁₋₃ alkyl (C₃₋₆)cycloalkyl; phenyl optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino; heterocyclyl optionally substituted by halo, nitro, cyano, C₁₋₆ alkyl, C₁₋₆ haloalkyl, C₁₋₆ alkoxy or C₁₋₆ haloalkoxy; formyl; C₁₋₆ alkylcarbonyl, C₁₋₆alkoxycarbonyl; C₁₋₆ alkylthiocarbonyl; C₁₋₆ alkoxythionocarbonyl; carbamoyl; C₁₋₆ alkylaminocarbonyl; di-C₁₋₆alkylaminocarbonyl; thiocarbamoyl; C₁₋₆ alkylaminothionocarbonyl; di-C₁₋₆ alkylaminothionocarbonyl; C₁₋₈ alkoxy; C₁₋₆ haloalkoxy; phenoxy optionally substituted by halogen, C₁₋₄ alkyl, C₁₋₄ alkoxy, C₁₋₄ haloalkyl, C₁₋₄ haloalkoxy, CN, NO₂ or phenyl; heteroaryloxy optionally substituted by halo, nitro, cyano, C₁₋₃ alkyl, C₁₋₃ haloalkyl, C₁₋₃ alkoxy or C₁₋₃ haloalkoxy; C₁₋₆ alkylcarbonyloxy, C₁₋₆ alkoxycarbonyloxy; C₁₋₆ alkylaminocarbonyloxy; di-C₁₋₆ alkylaminocarbonyloxy; C₁₋₆ alkylaminothionocarbonyloxy; di-C₁₋₆ alkylaminothionocarbonyloxy; C₁₋₈ alkylthio; C₁₋₆ haloalkylthio; arylthio or heteroarylthio, wherein the aryl or heteroaryl ring is optionally substituted by halogen, C₁₋₄ alkyl, C₁₋₄ alkoxy, C₁₋₄ haloalkyl, C₁₋₄ haloalkoxy, CN, NO₂ or phenyl; C₁₋₆ alkylcarbonylthio; C₁₋₆ alkylaminocarbonylthio; di-C₁₋₆ alkylaminocarbonylthio; di(C₁₋₈)alkylamino; C₁₋₆ alkylcarbonylamino; C₁₋₆ alkoxycarbonylamino; C₁₋₆ alkylaminocarbonylamino; di-C₁₋₆ alkylaminocarbonylamino; aminothionocarbonylamino; C₁₋₆ alkylaminothionocarbonylamino; di-C₁₋₆ alkylaminothionocarbonylamino; or 2 adjacent groups R⁴ together with the carbon atoms to which they are attached form a 4-, 5-, 6- or 7-membered carbocylic or heterocyclic ring, wherein said ring is optionally substituted by halogen.

8. The method according to claim 7, wherein each R⁴ is independently selected from fluoro, C₁₋₄alkyl and C₁₋₄haloalkyl.

9. The method according to claim 8, wherein n is 1 or 2.

10. The method according to claim 1, wherein R⁹ is: hydrogen; C₁₋₆ alkyl; C₁₋₆ cyanoalkyl; C₁₋₆ haloalkyl; C₃₋₇ cycloalkyl(C₁₋₄)alkyl; C₁₋₆ alkoxy(C₁₋₆)alkyl; aryl(C₁₋₆)alkyl wherein the aryl group is optionally substituted by halo, nitro, cyano, C₁₋₆ alkyl, C₁₋₆ haloalkyl, C₁₋₆ alkoxy, C₁₋₆ haloalkoxy, C₁₋₆ alkylsulfonyl, C₁₋₆ alkylsulfinyl, C₁₋₆ alkylthio, C₁₋₆ alkoxycarbonyl, C₁₋₆ alkylcarbonylamino or arylcarbonyl; C₂₋₆ alkylcarbonyl, phenylcarbonyl, wherein the phenyl group is optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino; C₁₋₆ alkoxycarbonyl; C(O)NR³⁴R³⁵, wherein R³⁴ and R³⁵ are each independently hydrogen, C₁₋₆ alkyl or C₁₋₆ haloalkyl or C₁₋₆ alkoxy(C₁₋₆)alkyl, or R³⁴ and R³⁵ together with the N atom to which they are attached form a 5-, 6-, or 7-membered ring containing an O or S atom; or R⁹ and R¹ together with the carbon atoms to which they are attached form a three to six membered ring optionally comprising one or two sulphur and/or one, or two non-adjacent, oxygen atom(s).

11. The method according to claim 1, wherein R¹⁰ and R⁷² are each independently hydrogen; hydroxyl; amino; cyano; C₁₋₆ alkyl; C₁₋₆ haloalkyl; C₁₋₆ alkoxy(C₁₋₆)alkyl; C₃₋₆-alkenyl; C₃₋₆alkinyl; aryl(C₁₋₃)alkyl or heteroaryl(C₁₋₃)alkyl, wherein the aryl or heteroaryl ring is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₃₋₅ cycloalkyl-C₁₋₃alkyl; C₃₋₅ cycloalkyl; aryl or heteroaryl, wherein the aryl or heteroaryl ring is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₁₋₆ alkylthio; C₁₋₆ haloalkylthio; C₁₋₆ alkyldithio; C₁₋₆ alkylthiosulfinyl; formyl; C₁₋₁₂ alkylcarbonyl; C₂₋₁₂ alkenylcarbonyl, where the alkenyl is substituted by C₁₋₆ alkoxycarbonyl or phenyl optionally substituted by halogen, C₁₋₃ alkyl, C₁₋₃ haloalkyl, C₁₋₃ alkoxy, cyano or nitro; C₃₋₆ cycloalkylcarbonyl; arylcarbonyl or heteroarylcarbonyl, wherein the aryl or heteroaryl ring is optionally substituted by halogen, C₁₋₄ alkyl, C₁₋₄ alkoxy, C₁₋₄ haloalkyl, C₁₋₄ haloalkoxy, cyano or nitro; naphthylcarbonyl; C₁₋₆ alkoxycarbonyl; C₁₋₆ alkylthiocarbonyl; C₁₋₆ alkylcarbonylamino; R²⁴R²⁵N—, R²⁴R²⁵N—S— or R²⁴R²⁵N-A-, wherein R²⁴ and R²⁵ are each independently hydrogen or C₁₋₆alkyl, and A is SO₂, C(O) or C(S).

12. The method according to claim 1, wherein R¹⁰ and R⁷² are independently: hydroxyl; amino; phenyl(C₂₋₃)alkyl, wherein the phenyl group is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; heteroaryl(C₁₋₃)alkyl, wherein the heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₃₋₅ cycloalkyl-C₁₋₃alkyl; heteroaryl optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl and C₁₋₆ alkoxycarbonyl; C₁₋₆ alkylthio; C₁₋₆ haloalkylthio; C₁₋₆ alkyldithio; C₁₋₆ alkylthiosulfinyl; C₇₋₁₂ alkylcarbonyl; C₇₋₁₂ alkenylcarbonyl; cinnamylcarbonyl, wherein the phenyl group is optionally substituted by halogen, C₁₋₃ alkyl, C₁₋₃ haloalkyl, C₁₋₃ alkoxy, cyano or nitro; aryl- or heteroaryl-carbonyl, wherein the aryl or heteroaryl moiety is substituted by halogen, C₁₋₄ alkyl, C₁₋₄ alkoxy, C₁₋₄ haloalkyl, C₁₋₄ haloalkoxy, cyano or nitro; napthylcarbonyl; (2,2-difluoro-benzo[1,3]dioxolyl)-carbonyl; C₁₋₆ alkylcarbonylamino; R²⁴R²⁵N—, R²⁴R²⁵N—S— or R²⁴R²⁵N-A-, wherein R²⁴ and R²⁵ are hydrogen or C₁₋₆alkyl and A is SO₂ or C(S).

13. The method according to claim 1, wherein R⁷³ is: hydrogen; C₁₋₄ alkoxy-C₁₋₄ alkyl; C₁₋₄ haloalkoxy-C₁₋₄ alkyl; cyano-C₁₋₄ alkyl; 2-tetrahydropyranyl; 2-tetrahydrofuranyl; vinyl; cinnamyl, wherein the phenyl is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl; C₃₋₆ alkenyl; C₃₋₆ alkinyl; aryl-di(C₁₋₄)alkylsilyl; C₁₋₄ alkyldi-arylsilyl; formyl; C₁₋₁₂ alkylcarbonyl, wherein the alkyl group is optionally substituted by one or more of halogen, C₁₋₆ alkoxy, C₁₋₆ alkylthio, C₁₋₆ alkoxycarbonyl, C₁₋₆ alkylcarbonyloxy, C₃₋₆ cycloalkyl, phenyl or phenoxy and said phenyl groups are optionally substituted by halogen, C₁₋₃ alkyl, C₁₋₃ haloalkyl, C₁₋₃alkoxy, cyano or nitro; C₂₋₁₂ alkenylcarbonyl, wherein the alkenyl is optionally substituted by halogen, C₁₋₆ alkoxycarbonyl or phenyl optionally substituted by halogen, C₁₋₃ alkyl, C₁₋₃ haloalkyl, C₁₋₃alkoxy, cyano or nitro; C₁₋₆ alkylthiocarbonyl; C₃₋₆ cycloalkylcarbonyl; adamantylcarbonyl; arylcarbonyl or heteroarylcarbonyl, wherein the aryl or heteroaryl is optionally substituted by halogen, C₁₋₄ alkyl, C₁₋₄ alkoxy, C₁₋₄ haloalkyl, C₁₋₄ haloalkoxy, cyano or nitro; C₁₋₆ alkoxycarbonyl; C₁₋₆ alkylthiocarbonyl; C₁₋₆ alkoxythionocarbonyl; C₁₋₆ alkylthiothionocarbonyl; benzyloxycarbonyl, phenoxycarbonyl or phenylthiocarbonyl and the phenyl groups are optionally substituted by halogen, C₁₋₃ alkyl, C₁₋₃ haloalkyl, C₁₋₃ alkoxy, cyano or nitro; R⁷⁸R⁷⁹N—C(O)— or R⁷⁸R⁷⁹N—C(S)—, wherein R⁷⁸ and R⁷⁹ are each independently hydrogen, C₁₋₆ alkyl, C₃₋₆ alkenyl, C₃₋₆ alkinyl or phenyl optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl.

14. The method according to claim 1, wherein R⁷³ is C₁₋₄ alkoxy-C₁₋₄ alkyl; C₁₋₄ haloalkoxy-C₁₋₄ alkyl; cyano-C₁₋₄ alkyl; 2-tetrahydropyranyl; 2-tetrahydrofuranyl; cinnamyl, wherein the phenyl group is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl; aryldi(C₁₋₄)alkylsilyl; C₁₋₄ alkyldiarylsilyl; C₁₋₁₂ alkylcarbonyl, wherein the alkyl group issubstituted by C₁₋₆ alkoxycarbonyl, C₁₋₆ alkylcarbonyloxy, C₃₋₆ cycloalkyl, phenyl or phenoxy and said phenyl groups are optionally substituted by halogen, C₁₋₃ alkyl, C₁₋₃ haloalkyl, C₁₋₃ alkoxy, cyano or nitro; C₃₋₆ cycloalkylcarbonyl; adamantylcarbonyl; arylcarbonyl or heteroarylcarbonyl, wherein said aryl or heteroaryl group is substituted by C₁₋₄ haloalkyl, C₁₋₄ haloalkylthio, di-C₁₋₄-alkylamino, benzoyloxymethyl, phenyl or phenylsulfonyl and said phenyl groups are optionally substituted by halogen, C₁₋₃ alkyl, C₁₋₃ haloalkyl, C₁₋₃ alkoxy, cyano or nitro; naphthylcarbonyl; (2,3-dihydro-benzofuranyl)carbonyl; (2,2-difluoro-benzo[1,3]dioxolyl)carbonyl; phenoxycarbonyl or phenylthiocarbonyl, wherein said phenyl groups are optionally substituted by halogen, C₁₋₃ alkyl, C₁₋₃ haloalkyl, C₁₋₃ alkoxy, cyano or nitro; C₂₋₁₂ alkenylcarbonyl, wherein the alkenyl is substituted by C₁₋₆ alkoxycarbonyl or phenyl and said phenyl group is optionally substituted by halogen, C₁₋₃ alkyl, C₁₋₃ haloalkyl, C₁₋₃ alkoxy, cyano or nitro; benzyloxycarbonyl, wherein the phenyl group is optionally substituted by halogen, C₁₋₃ alkyl, C₁₋₃ haloalkyl, C₁₋₃ alkoxy, cyano or nitro; R⁷³R⁷⁹N—C(O)— or R⁷⁸R⁷⁹N—C(S)—, wherein R⁷⁸ and R⁷⁹ are independently hydrogen; C₃₋₆ alkenyl or phenyl and said phenyl group is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl; or R⁷⁸ and R⁷⁹ together with the N atom to which they are attached form a 5-, 6- or 7-membered ring optionally comprising one or two further hetero atoms selected from O, N or S and wherein said ring is optionally substituted by one to four C₁₋₆ alkyl groups.

15. The method according to claim 1, wherein L is a direct bond, R⁴⁶, R⁴⁷, R⁴⁸ and R⁴⁹ are hydrogen, and least one of R¹⁰ and R⁷² is hydrogen.

16. The method according to claim 1, wherein T is a benzene ring, Y is CH₂ or CH₂CH₂, L is a direct bond, and R¹, R², R⁹, R⁴⁶, R⁴⁷, R⁴³ and R⁴⁹ are each hydrogen.

17. A method according to claim 1, wherein T and Y form an indane ring system, and at least one of R¹⁰ and R⁷² is hydrogen.

18. The method according to claim 1, wherein at least one of R¹, R², R⁵, R⁶, R⁷, R⁸ or R⁹ is other than hydrogen.

19. A compound of formula (I) as defined in claim 1.

20. The compound according to claim 19, wherein T is benzene, Y is CR⁵R⁶ or CR⁵R⁶CR⁷R⁸, L is a direct bond and each of R⁴⁶, R⁴⁷, R⁴⁸ and R⁴⁹ is hydrogen.

21. The compound according to claim 19, wherein R⁷³ is: hydrogen; C₁₋₄ alkoxy-C₁₋₄ alkyl; C₁₋₄ haloalkoxy-C₁₋₄ alkyl; cyano-C₁₋₄ alkyl; 2-tetrahydropyranyl; 2-tetrahydrofuranyl; vinyl; cinnamyl, wherein the phenyl is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl; C₃₋₆ alkenyl; C₃₋₆ alkinyl; aryl-di(C₁₋₄)alkylsilyl; C₁₋₄ alkyldi-arylsilyl; formyl; C₁₋₁₂ alkylcarbonyl, wherein the alkyl group is optionally substituted by one or more of halogen, C₁₋₆ alkoxy, C₁₋₆ alkylthio, C₁₋₆alkoxycarbonyl, C₁₋₆ alkylcarbonyloxy, C₃₋₆ cycloalkyl, phenyl or phenoxy and said phenyl groups are optionally substituted by halogen, C₁₋₃ alkyl, C₁₋₃ haloalkyl, C₁₋₃ alkoxy, cyano or nitro; C₂₋₁₂ alkenylcarbonyl, wherein the alkenyl is optionally substituted by halogen, C₁₋₆ alkoxycarbonyl or phenyl optionally substituted by halogen, C₁₋₃ alkyl, C₁₋₃ haloalkyl, C₁₋₃ alkoxy, cyano or nitro; C₁₋₆ alkylthiocarbonyl; C₃₋₆ cycloalkylcarbonyl; adamantylcarbonyl; arylcarbonyl or heteroarylcarbonyl, wherein the aryl or heteroaryl is optionally substituted by halogen, C₁₋₄ alkyl, C₁₋₄ alkoxy, C₁₋₄ haloalkyl, C₁₋₄ haloalkoxy, cyano or nitro; C₁₋₆ alkoxycarbonyl; C₁₋₆ alkylthiocarbonyl; C₁₋₆ alkoxythionocarbonyl; C₁₋₆ alkylthiothionocarbonyl; benzyloxycarbonyl, phenoxycarbonyl or phenylthiocarbonyl and the phenyl groups are optionally substituted by halogen, C₁₋₃ alkyl, C₁₋₃ haloalkyl, C₁₋₃ alkoxy, cyano or nitro; R⁷⁸R⁷⁹N—C(O)— or R⁷⁸R⁷⁹N—C(S)—, wherein R⁷⁸ and R⁷⁹ are each independently hydrogen, C₁₋₆ alkyl, C₃₋₆ alkenyl, C₃₋₆ alkinyl or phenyl optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl.

22. The compound according to claim 19, wherein R⁷³ is: C₁₋₄ alkoxy-C₁₋₄ alkyl; C₁₋₄ haloalkoxy-C₁₋₄ alkyl; cyano-C₁₋₄ alkyl; 2-tetrahydropyranyl; 2-tetrahydrofuranyl; cinnamyl, wherein the phenyl group is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl; aryl-di(C₁₋₄)alkylsilyl, (C₁₋₄)alkyldiarylsilyl; C₁₋₁₂ alkylcarbonyl, wherein the alkyl group is substituted by C₁₋₆ alkoxycarbonyl, C₁₋₆ alkylcarbonyloxy, C₃₋₆ cycloalkyl, phenyl or phenoxy and said phenyl groups are optionally substituted by halogen, C₁₋₃ alkyl, C₁₋₃ haloalkyl, C₁₋₃ alkoxy, cyano or nitro; C₃₋₆ cycloalkylcarbonyl; adamantylcarbonyl; arylcarbonyl or heteroaryl-carbonyl, wherein the aryl or heteroaryl is substituted by C₁₋₄ haloalkyl, C₁₋₄ haloalkylthio, di-C₁₋₄-alkylamino, benzoyloxymethyl, phenyl or phenylsulfonyl and said phenyl groups are optionally substituted by halogen, C₁₋₃ alkyl, C₁₋₃ haloalkyl, C₁₋₃alkoxy, cyano or nitro; (2,3-dihydro-benzofuranyl)carbonyl; (2,2-difluoro-benzo[1,3]dioxolyl)-carbonyl; phenoxycarbonyl or phenylthiocarbonyl, wherein the phenyl groups are optionally substituted by halogen, C₁₋₃ alkyl, C₁₋₃ haloalkyl, C₁₋₃alkoxy, cyano or nitro; C₂₋₁₂ alkenylcarbonyl, wherein the alkenyl is substituted by C₁₋₆ alkoxycarbonyl or phenyl and said phenyl group is optionally substituted by halogen, C₁₋₃ alkyl, C₁₋₃ haloalkyl, C₁₋₃alkoxy, cyano or nitro; benzyloxycarbonyl, wherein the phenyl group is optionally substituted by halogen, C₁₋₃ alkyl, C₁₋₃ haloalkyl, C₁₋₃ alkoxy, cyano or nitro; R⁷⁸R⁷⁹N—C(O)— or R⁷⁸R⁷⁹N—C(S)—, wherein R⁷⁸ and R⁷⁹ are each independently hydrogen, C₃₋₆ alkenyl or phenyl and said phenyl group is optionally substituted by halogen, C₁₋₄ alkyl, C₁₋₄ alkoxy, C₁₋₄ haloalkyl, C₁₋₄ haloalkoxy, cyano, nitro, aryl, heteroaryl, C₁₋₆ alkylsulfonyl or C₁₋₆ alkoxycarbonyl; or R⁷⁸ and R⁷⁹ together with the N atom to which they are attached form a 5-, 6- or 7-membered ring optionally comprising one or two further hetero atoms selected from O, N or S and wherein said ring is optionally substituted by one to four C₁₋₆ alkyl groups.

23. The compound according to claim 19, wherein R¹⁰ and R⁷² are each independently hydrogen; hydroxyl; amino; cyano; C₁₋₆ alkyl; C₁₋₆ haloalkyl; C₁₋₆ alkoxy(C₁₋₆)alkyl; C₃₋₆-alkenyl; C₃₋₆alkinyl; aryl(C₁₋₃)alkyl or heteroaryl(C₁₋₃)alkyl, wherein the aryl or heteroaryl ring is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₃₋₅ cycloalkyl-C₁₋₃alkyl; C₃₋₅ cycloalkyl; aryl or heteroaryl, wherein the aryl or heteroaryl ring is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₁₋₆ alkylthio; C₁₋₆ haloalkylthio; C₁₋₆ alkyldithio; C₁₋₆ alkylthiosulfinyl; formyl; C₁₋₁₂ alkylcarbonyl; C₂₋₁₂ alkenylcarbonyl, where the alkenyl is substituted by C₁₋₆ alkoxycarbonyl or phenyl optionally substituted by halogen, C₁₋₃ alkyl, C₁₋₃ haloalkyl, C₁₋₃ alkoxy, cyano or nitro; C₃₋₆ cycloalkylcarbonyl; arylcarbonyl or heteroarylcarbonyl, wherein the aryl or heteroaryl ring is optionally substituted by halogen, C₁₋₄ alkyl, C₁₋₄ alkoxy, C₁₋₄ haloalkyl, C₁₋₄ haloalkoxy, cyano or nitro; naphthylcarbonyl; C₁₋₆ alkoxycarbonyl; C₁₋₆ alkylthiocarbonyl; C₁₋₆ alkylcarbonylamino; R²⁴R²⁵N—, R²⁴R²⁵N—S— or R²⁴R²⁵N-A-, wherein R²⁴ and R²⁵ are each independently hydrogen or C₁₋₆alkyl, and A is SO₂, C(O) or C(S).

24. The compound according to claim 19, wherein R¹⁰ and R⁷² are each independently hydroxyl; amino; phenyl(C₂₋₃)alkyl, wherein said phenyl group is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; heteroaryl(C₁₋₃)alkyl, wherein the heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₃₋₅ cycloalkyl-C₁₋₃alkyl; heteroaryl, wherein the heteroaryl is optionally substituted by halogen, C₁₋₄ alkyl, C₁₋₄ alkoxy, C₁₋₄ haloalkyl, C₁₋₄ haloalkoxy, cyano, nitro, C₁₋₆ alkylsulfonyl or C₁₋₆ alkoxycarbonyl; C₁₋₆ alkylthio; C₁₋₆ haloalkylthio; C₁₋₆ alkyldithio; C₁₋₆ alkylthiosulfinyl; C₇₋₁₂ alkylcarbonyl; C₇₋₁₂ alkenylcarbonyl; cinnamylcarbonyl, wherein the phenyl group is optionally substituted by halogen, C₁₋₃ alkyl, C₁₋₃ haloalkyl, C₁₋₃ alkoxy, cyano or nitro; arylcarbonyl or heteroarylcarbonyl, wherein the aryl or heteroaryl is substituted by halogen, C₁₋₄ alkyl, C₁₋₄ alkoxy, C₁₋₄ haloalkyl, C₁₋₄ haloalkoxy, cyano or nitro; naphthylcarbonyl; (2,2-difluoro-benzo[1,3]dioxolyl)-carbonyl; C₁₋₆ alkylcarbonylamino; R²⁴R²⁵N—, R²⁴R²⁵N—S— or R²⁴R²⁵N-A-, wherein R²⁴ and R²⁵ are each independently hydrogen or C₁₋₆ alkyl and A is SO₂ or C(S).

25. The compound according to claim 19, wherein n is 1, 2 or 3 and each R⁴ is independently: halogen; cyano; C₁₋₈ alkyl; C₁₋₈ haloalkyl; cyano(C₁₋₆)alkyl; C₁₋₃ alkoxy(C₁₋₃)alkyl; C₂₋₆ alkynyl; C₃₋₆ cycloalkyl; C₁₋₃ alkyl (C₃₋₆)cycloalkyl; phenyl optionally substituted by halogen, C₁₋₄ alkyl, C₁₋₄ alkoxy, C₁₋₄ haloalkyl, C₁₋₄ haloalkoxy, CN, NO₂, aryl, heteroaryl, amino or dialkylamino; heterocyclyl optionally substituted by halo, nitro, cyano, C₁₋₆ alkyl, C₁₋₆ haloalkyl, C₁₋₆ alkoxy or C₁₋₆ haloalkoxy; formyl; C₁₋₆ alkylcarbonyl, C₁₋₆alkoxycarbonyl; C₁₋₆ alkylthiocarbonyl; C₁₋₆ alkoxythionocarbonyl; carbamoyl; C₁₋₆ alkylaminocarbonyl; di-C₁₋₆alkylaminocarbonyl; thiocarbamoyl; C₁₋₆ alkylaminothionocarbonyl; di-C₁₋₆ alkylaminothionocarbonyl; C₁₋₈ alkoxy; C₁₋₆ haloalkoxy; phenoxy optionally substituted by halogen, C₁₋₄ alkyl, C₁₋₄ alkoxy, C₁₋₄ haloalkyl, C₁₋₄ haloalkoxy, CN, NO₂ or phenyl; heteroaryloxy optionally substituted by halo, nitro, cyano, C₁₋₃ alkyl, C₁₋₃ haloalkyl, C₁₋₃ alkoxy or C₁₋₃ haloalkoxy; C₁₋₆ alkylcarbonyloxy, C₁₋₆ alkoxycarbonyloxy; C₁₋₆ alkylaminocarbonyloxy; di-C₁₋₆ alkylaminocarbonyloxy; C₁₋₆ alkylaminothionocarbonyloxy; di-C₁₋₆ alkylaminothionocarbonyloxy; C₁₋₈ alkylthio; C₁₋₆ haloalkylthio; arylthio or heteroarylthio, wherein the aryl or heteroaryl ring is optionally substituted by halogen, C₁₋₄ alkyl, C₁₋₄ alkoxy, C₁₋₄ haloalkyl, C₁₋₄ haloalkoxy, CN, NO₂ or phenyl; C₁₋₆ alkylcarbonylthio; C₁₋₆ alkylaminocarbonylthio; di-C₁₋₆ alkylaminocarbonylthio; di(C₁₋₈)alkylamino; C₁₋₆ alkylcarbonylamino; C₁₋₆ alkoxycarbonylamino; C₁₋₆ alkylaminocarbonylamino; di-C₁₋₆ alkylaminocarbonylamino; aminothionocarbonylamino; C₁₋₆ alkylaminothionocarbonylamino; di-C₁₋₆ alkylaminothionocarbonylamino; or 2 adjacent groups R⁴ together with the carbon atoms to which they are attached form a 4-, 5-, 6- or 7-membered carbocylic or heterocyclic ring, wherein said ring is optionally substituted by halogen.

26. The compound according to claim 25 wherein each R⁴ is independently selected from fluoro, C₁₋₄alkyl and C₁₋₄haloalkyl.

27. The compound according to claim 26 wherein n is 1 or 2.

28. The compound according to claim 19, wherein n is 1, 2, 3 and at least one R⁴ is selected from fluoro, methyl, fluoromethyl, difluoromethyl and trifluoromethyl.

29. The compound according to claim 19, wherein Y is O, S(O)ₘ, NR³, SO₂—NR³, NR³—SO₂, NR³—O, O—NR³, O—CR⁷R⁸, S(O)ₘ—CR⁷R⁸, NR³—CR⁷R⁸, CR⁵R⁶—O, CR⁵R⁶S(O)ₘ or CR⁵R⁶—NR³ and wherein R³, R⁵, R⁶, R⁷ and R⁸ are as defined in claim 1.

30. The compound according to claim 1, wherein T is a 5- or 6-membered heteroaromatic ring, wherein the ring members are each independently CH, S, N, NR⁴, O, or CR⁴, wherein R⁴ is as defined in claim 1, provided that there are no more than one O or S atom present in the ring.

31. The compound according to claim 19, wherein T is a benzene ring.

32. The compound according to claim 19, wherein T and Y complete an indane ring system, and at least one of R¹⁰ and R⁷² is hydrogen.

33. The compound according to claim 19 wherein L is a direct bond, R⁴⁶, R⁴⁷, R⁴⁸, R⁴⁹ are hydrogen, and at least one of R¹⁰ and R⁷² is hydrogen.

34. The compound according to claim 19, wherein at least one of R¹, R², R⁵, R⁶, R⁷, R⁸ or R⁹ is other than hydrogen.

35. A process for making a compound as defined in claim 19, wherein R¹⁰ is hydrogen, the process comprising:
reacting a compound of the formula (III) 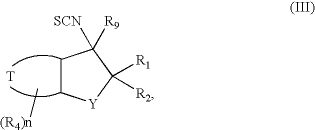
wherein T, Y, R¹, R², R⁴, R⁹ and n are as defined as in claim 1, with a compound of formula (IVa) 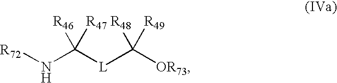
wherein L, R⁴⁶, R⁴⁷, R⁴⁸, R⁴⁹, R⁷² and R⁷³ are as defined in claim 1.

36. A process for making a compound defined in claim 19 wherein R¹⁰ is other than hydrogen, the process comprising:
reacting a compound of the formula (IIIa) 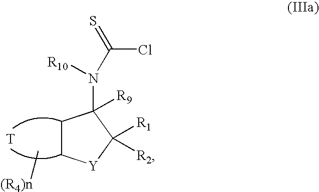
wherein T, Y, R¹, R², R⁴, R⁹, R¹⁰ and n are as defined as in claim 1, with a compound of formula (IVa) 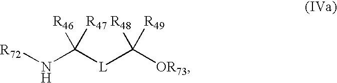
wherein L, R⁴⁶, R⁴⁷, R⁴⁸, R⁴⁹, R⁷² and R⁷³ are as defined in claim 1.

37. An insecticidal, acaricidal, nematicidal or molluscicidal composition comprising an effective amount of a compound as defined in claim 19.
